(19) United States
(12) Patent Application Publication
(10) Pub. No.: US 2016/0333019 A1
(43) Pub. Date: Nov. 17, 2016

(54) SYNTHESIS AND FORMULATIONS OF PORPHYRIN COMPOUNDS

(71) Applicant: Aeolus Sciences, Inc., Mission Viejo, CA (US)

(72) Inventors: Jason Brittain, El Cajon, CA (US); Chris Stanley, San Clemente, CA (US); Alexander Kolchinski, Winchester, MA (US); Mahmoud Mirmehrabi, Halifax (CA); Duane Bell, Maynard, MA (US)

(73) Assignee: Aeolus Sciences, Inc., Mission Viejo, CA (US)

(21) Appl. No.: 15/159,551

(22) Filed: May 19, 2016

Related U.S. Application Data
(63) Continuation of application No. PCT/US2014/066923, filed on Nov. 21, 2014.
(60) Provisional application No. 61/907,664, filed on Nov. 22, 2013.

(51) Int. Cl. C07D 487/22; C07F 13/00

(52) U.S. Cl. CPC C07D 487/22; C07F 13/005

(57) ABSTRACT

Provided herein, inter alia, are methods of synthesizing and formulating porphyrins, including manganese containing porphyrins. Also provided herein are pharmaceutical compositions and crystals of porphyrins achieved using the methods described herein.

### RELATED APPLICATIONS

This application is a continuation of International Application No. PCT/US2014/066923, filed Nov. 21, 2014, which claims the benefit of U.S. Provisional Application No. 61/907,664, filed Nov. 22, 2013, each of which is hereby incorporated by reference in its entirety and for all purposes.

### BACKGROUND OF THE INVENTION

Methods used in the art for synthesizing porphyrins, including manganese porphyrins, suffer from poor yields and impure product. Current methods are undesirable for synthesizing prophyrin products since yields and purity vary. Accordingly, there is a need in the art for methods of synthesizing and formulating porphyrins, including manganese porphyrins, in greater yields with higher purity. Provided herein are solutions to these and other problems in the art.

### BRIEF SUMMARY OF THE INVENTION

Provided herein, inter alia, are methods of synthesizing and formulating porphyrins, including manganese containing porphyrins. Also provided herein are pharmaceutical compositions and crystals of porphyrins achieved using the methods described herein.

In a first aspect is a method for synthesizing a substituted porphyrin having the formula

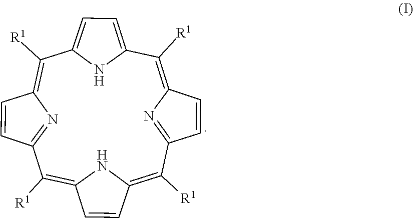

R¹ is substituted or unsubstituted heterocycloalkyl or substituted or unsubstituted heteroaryl. The method includes contacting a pyrrole with an R¹-substituted aldehyde. The contacting is performed in a solvent system that includes a positive azeotrope. The pyrrole is allowed to react with the R¹-substituted aldehyde in the solvent system under azeotropic distillation conditions, thereby forming a substituted-porphyrinogen. The substituted-porphyrinogen is oxidized, thereby synthesizing a substituted porphyrin having formula (I).

In another aspect, a method is provided for synthesizing a compound having the formula:

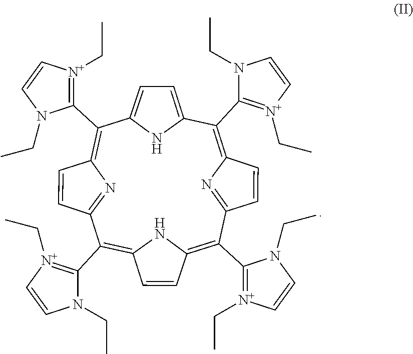 [SMILES: CCn1cc[n+](CC)c1-c1c2nc(c(-c3n(CC)cc[n+]3CC)c3ccc([nH]3)c(-c3n(CC)cc[n+]3CC)c3nc(c(-c4n(CC)cc[n+]4CC)c4ccc1[nH]4)C=C3)C=C2]

The method includes contacting with an ethylating agent a compound having the formula

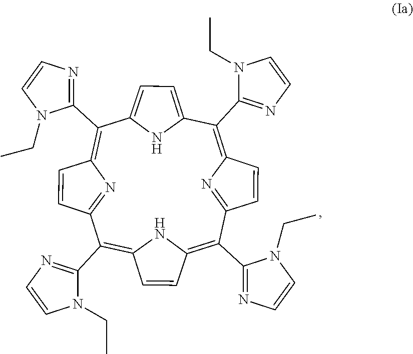 [SMILES: CCn1ccnc1-c1c2nc(c(-c3nccn3CC)c3ccc([nH]3)c(-c3nccn3CC)c3nc(c(-c4nccn4CC)c4ccc1[nH]4)C=C3)C=C2]

thereby synthesizing a compound of formula (II).

In another aspect, a method is provided for synthesizing a hydrate compound having the formula

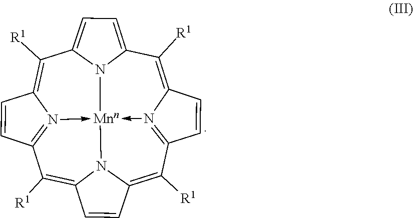

In Formula (III), R¹ is substituted or unsubstituted heterocycloalkyl or substituted or unsubstituted heteroaryl and n is 2 or 3. The method includes contacting a compound of formula:

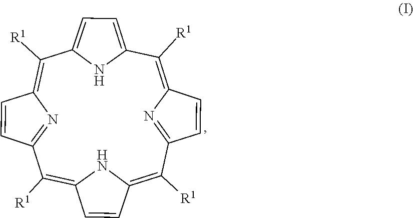

with over about 2 equivalents of a Mn(III) salt in a solvent, thereby forming a reaction mixture. The reaction mixture is heated thereby synthesizing a compound of formula (III). The compound of formula (III) is hydrated thereby forming a hydrate of compound (III).

In another aspect is a container having a plurality compounds. The plurality of compounds have the formula:

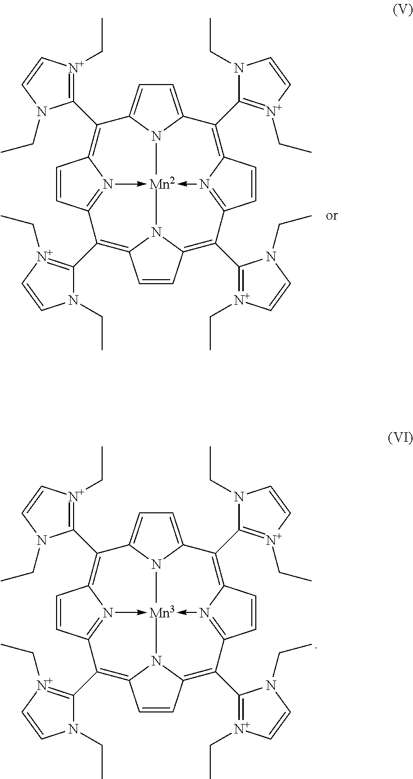

In another aspect, a pharmaceutical formulation is provided that includes water and a compound having the formula:

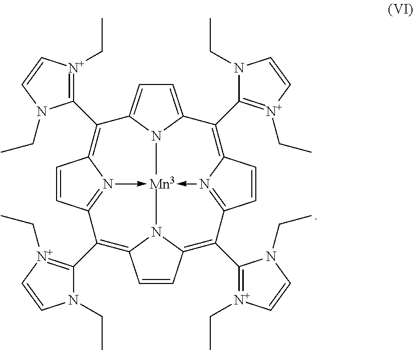

In another aspect, is provided a crystal that includes a compound having the formula:

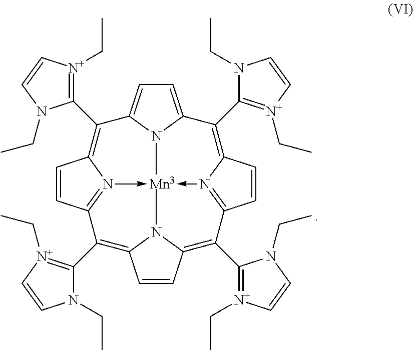

In another aspect is a method for purifying a compound of formula:

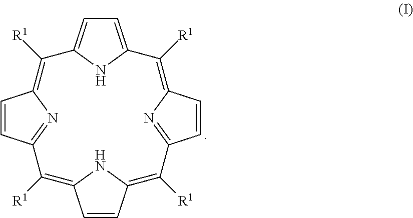

The method includes combining a compound of formula (I) and a purification solvent in a reaction vessel thereby forming a purification mixture. The compound is insoluble in the purification solvent. The purification mixture is heated. The purification mixture is cooled. The purification mixture is filtered, thereby purifying a compound of formula (I).

In another aspect is a method for purifying a compound having the formula:

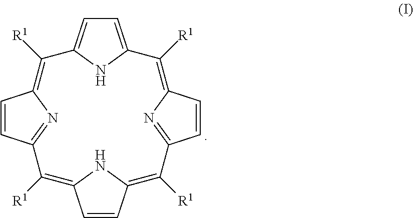

The method includes dissolving a compound of formula (I) in a purifying solvent in a reaction vessel to form a purifying mixture. The purifying mixture is heated. The purifying mixture is cooled. The purifying mixture is filtered thereby purifying a compound of formula (I).

In another aspect, is provided a crystalline form of [5,10,15,20-tetrakis(1,3-diethylimidazolium-2-yl)porphyrinato]manganese(III) chloride hydrate complex. The crystalline form is characterized by an x-ray powder diffraction spectrum. The x-ray powder diffraction spectrum includes angle 2θ peaks at about 6.9±0.2, 8.2±0.2, 9.5±0.2, 11.4±0.2, 12.8±0.2, 14.5±0.2, 15.0±0.2, 16.1±0.2, 16.3±0.2, 18.1±0.2, 20.3±0.2, 23.5±0.2, 24.8±0.2, 25.6±0.2, 26.5±0.2, and 29.2±0.2. The x-ray powder diffraction spectrum is obtained using a Cu Kα radiation source (1.54 Å).

In another aspect, is provided a crystalline form of [5,10,15,20-tetrakis(1,3-diethylimidazolium-2-yl)porphyrinato]manganese(III) chloride hydrate complex. The crystalline form is characterized by an x-ray powder diffraction spectrum. The x-ray powder diffraction spectrum includes d spacings at about 12.85, 10.82, 9.28, 7.78, 6.91, 6.11, 5.91, 5.49, 5.42, 4.89, 4.37, 3.78, 3.58, 3.47, 3.36, and 3.06. The x-ray powder diffraction spectrum is obtained using a Cu Kα radiation source (1.54 Å).

In another aspect, is provided a crystalline form of [5,10,15,20-tetrakis(1,3-diethylimidazolium-2-yl)porphyrinato]manganese(III) chloride hydrate complex. The crystalline form is characterized by an x-ray powder diffraction spectrum. The x-ray powder diffraction spectrum includes angle 2θ peaks at about 26.2±0.2, 22.9±0.2, 20.0±0.2, 18.6±0.2, 15.2±0.2, 13.7±0.2, 13.5±0.2, 13.0±0.2, 12.4±0.2, 11.4±0.2, 10.6±0.2, 8.9±0.2, 6.8±0.2, and 6.0±0.2. The x-ray powder diffraction spectrum is obtained using a Cu Kα radiation source (1.54 Å).

In another aspect, is provided a crystalline form of [5,10,15,20-tetrakis(1,3-diethylimidazolium-2-yl)porphyrinato]manganese(III) chloride hydrate complex. The crystalline form is characterized by an x-ray powder diffraction spectrum. The x-ray powder diffraction spectrum includes d spacings at about 14.74, 12.93, 9.99, 8.34, 7.74, 7.14, 6.80, 6.55, 6.45, 5.83, 4.78, 4.43, 3.89, and 3.40. The x-ray powder diffraction spectrum is obtained using a Cu Kα radiation source (1.54 Å).

In another aspect, is provided a crystalline form of [5,10,15,20-tetrakis(1,3-diethylimidazolium-2-yl)porphyrinato]manganese(III) chloride hydrate complex. The crystalline form is characterized by an x-ray powder diffraction spectrum. The x-ray powder diffraction spectrum includes angle 2θ peaks at about 27.7±0.2, 26.6±0.2, 19.9±0.2, 15.4±0.2, 14.7±0.2, 11.6±0.2, 10.1±0.2, 8.6±0.2, and 6.9±0.2. The x-ray powder diffraction spectrum is obtained using a Cu Kα radiation source (1.54 Å).

In another aspect, is provided a crystalline form of [5,10,15,20-tetrakis(1,3-diethylimidazolium-2-yl)porphyrinato]manganese(III) chloride hydrate complex. The crystalline form is characterized by an x-ray powder diffraction spectrum. The x-ray powder diffraction spectrum includes d spacings at about 12.89, 10.27, 8.79, 7.60, 6.04, 5.74, 4.45, 3.35, and 3.22. The x-ray powder diffraction spectrum is obtained using a Cu Kα radiation source (1.54 Å).

In another aspect, is provided a crystalline form of [5,10,15,20-tetrakis(1,3-diethylimidazolium-2-yl)porphyrinato]manganese(III) chloride hydrate complex. The crystalline form is characterized by an x-ray powder diffraction spectrum. The x-ray powder diffraction spectrum includes angle 2θ peaks at about 29.5±0.2, 27.3±0.2, 26.3±0.2, 24.7±0.2, 23.5±0.2, 22.5±0.2, 21.6±0.2, 20.5±0.2, 19.3±0.2, 17.7±0.2, 13.1±0.2, 10.8±0.2, 9.9±0.2, 8.5±0.2, and 6.0±0.2. The x-ray powder diffraction spectrum is obtained using a Cu Kα radiation source (1.54 Å).

In another aspect, is provided a crystalline form of [5,10,15,20-tetrakis(1,3-diethylimidazolium-2-yl)porphyrinato]manganese(III) chloride hydrate complex. The crystalline form is characterized by an x-ray powder diffraction spectrum. The x-ray powder diffraction spectrum includes angle 2θ peaks at about 23.5±0.2, 9.1±0.2, 6.9±0.2, and 5.8±0.2. The x-ray powder diffraction spectrum is obtained using a Cu Kα radiation source (1.54 Å).

In another aspect, is provided a crystalline form of [5,10,15,20-tetrakis(1,3-diethylimidazolium-2-yl)porphyrinato]manganese(III) chloride hydrate complex. The crystalline form is characterized by an x-ray powder diffraction spectrum. The x-ray powder diffraction spectrum includes d spacings at about 15.12, 12.74, 9.75, and 3.78. The x-ray powder diffraction spectrum is obtained using a Cu Kα radiation source (1.54 Å).

In another aspect, is provided a crystalline form of [5,10,15,20-tetrakis(1,3-diethylimidazolium-2-yl)porphyrinato]manganese(III) chloride hydrate complex. The crystalline form is characterized by an x-ray powder diffraction spectrum. The x-ray powder diffraction spectrum includes angle 2θ peaks at about 27.7±0.2, 23.6±0.2, 23.1±0.2, 20.7±0.2, 6.9±0.2, and 5.8±0.2. The x-ray powder diffraction spectrum is obtained using a Cu Kα radiation source (1.54 Å).

In another aspect, is provided a crystalline form of [5,10,15,20-tetrakis(1,3-diethylimidazolium-2-yl)porphyrinato]manganese(III) chloride hydrate complex. The crystalline form is characterized by an x-ray powder diffraction spectrum. The x-ray powder diffraction spectrum includes angle 2θ peaks at about 27.7±0.2, 20.7±0.2, 13.8±0.2, 11.4±0.2, 9.5±0.2, 8.2±0.2, and 6.9±0.2. The x-ray powder diffraction spectrum is obtained using a Cu Kα radiation source (1.54 Å).

In another aspect, is provided a crystalline form of [5,10,15,20-tetrakis(1,3-diethylimidazolium-2-yl)porphyrinato]manganese(III) chloride hydrate complex. The crystalline form is characterized by an x-ray powder diffraction spectrum. The x-ray powder diffraction spectrum includes d spacings at about 12.84, 10.83, 9.26, 7.77, 6.43, 4.29, and 3.22. The x-ray powder diffraction spectrum is obtained using a Cu Kα radiation source (1.54 Å).

### BRIEF DESCRIPTION OF THE DRAWINGS

FIG. 1. General synthetic scheme for synthesizing compounds disclosed herein: Porphyrin (I) is synthesized using pyrrole as starting material in a propionic acid and toluene solvent system, followed by alkylation to form the imidazolium derivative which is then titrated with Mn(III) salt.

FIG. 2. X-ray powder diffraction spectrum overlay of interconversion to form I: The relative humidity of the lab was at 54% at the time of filtration; the wet cake was washed with acetonitrile followed by XRPD analysis which conformed to Form I was then dried on a XRPD plate with dome in the over at 40° C., under vacuum for overnight wherein the sample holder was capped while in the oven followed by XRPD analysis; the resulting solid was a Form III which converted to Form I after opening and allowing the solid to dry and be exposed to ambient at RH of 54%.

FIG. 3. Differential Scanning Calorimetry (DSC) of form I at 115° C.; Form I was heated to 115° C. (which is just after the first peak) then cooled to room temperature under nitrogen before transferring into a XRPD sample holder with dome.

FIG. 4. X-ray powder diffraction spectrum of form I at 115° C.: The XRPD was taken after cooling to room temperature resulting in Form III; further exposure of the solid relative humidity of 70-80% for 15 minutes followed by XRPD analysis which showed Form I and apparent reversibility.

FIG. 5. Differential Scanning Calorimetry (DSC) of form I at 180° C.: Form I was heated to higher temperature of 180° C. which was the end point of the second endothermic peak; The sample was cooled to room temperature under nitrogen before transferring into a XRPD sample holder with dome.

FIG. 6. X-ray powder diffraction spectrum of form I at 180° C.: The XRPD was taken after cooling to RT and results in mainly amorphous solid with some peaks (after this point, the sample melts/degrades); the solid was exposed to relative humidity of 70-80% for 15 minutes followed by XRPD analysis showing Form I and apparent reversibility.

FIG. 7. FIG. 7 depicts flowchart of polymorph formation and interconversion for formula (VI).

FIG. 8. Competitive slurry of various forms at 25° C.: Mixture of six crystal forms (I, II, III, V, VI and VII) 1 were slurried in three different solvents (acetonitrile, acetonitrile:water (98:2) and ethyl acetate), at 25±2° C. for 5 days followed by filtration under nitrogen inert conditions (about 20 mg of each polymorph added to the vials); after filtration, the cake was washed with the same solvent as the one used in the slurry and placed on a sample holder and sealed using the X-ray transparent dome and analyzed using XRPD after which the cap was then removed and solid was dried at 45° C. and under vacuum for half a day before sealing under nitrogen inert environment and analyzed by XRPD; the dry sample was exposed to about 50% relative humidity for 30 minutes followed by XRPD analysis showing form I as final product.

FIG. 9. Overlay of 7 polymorphs of compound (VI): the different polymorphs have varying XRPD signatures but using the conditions described herein convert to form I.

FIG. 10. X-ray powder diffraction spectrum of form I: form I appears to be the stable under ambient conditions and at a relative humidity of as low as 15%.

FIG. 11. Differential Scanning Calorimetry (DSC) of form I: DSC shows peaks at approximately 82° C., 143° C. and 274° C.

FIG. 12. FTIR of form I showing expected peaks of functional groups.

FIG. 13. FTIR of hydrated compound (VI) shows expected shifting of peaks resulting from hydration.

FIG. 14. X-ray powder diffraction spectrum of hydrated compound (VI) shows shifting and broadening of peaks associated with the hydration of the compound.

FIG. 15. X-ray powder diffraction spectrum of form II (a silicon plate with dome was used to prevent exposure to ambient).

FIG. 16. X-ray powder diffraction spectrum of form III (a silicon plate with dome was used to prevent exposure to ambient).

FIG. 17. X-ray powder diffraction spectrum of form IV (a silicon plate with dome was used to prevent exposure to ambient).

FIG. 18. X-ray powder diffraction spectrum of form V (a silicon plate with dome was used to prevent exposure to ambient).

FIG. 19. X-ray powder diffraction spectrum of form VI.

FIG. 20. X-ray powder diffraction spectrum of form VII.

FIG. 21. ¹H NMR for compound of formula (I): apart from residual solvent peaks the NMR data for samples prepared under N₂ and in air (lower) were nearly identical indicating that air oxidation is not necessary to synthesize the porphyrin.

FIG. 22. UV-visible spectrum for oxidation of compound (V) to (VI) after about 20 minutes: titration with about 3 equivalents of Mn(III) salt indicated minimal presence of the Mn(II) form and minimal reoxidation.

FIG. 23. UV-vis studies of oxidation of Mn(II) in the degassed water-0.1% TFA: UV-vis absorptions characteristic for the reduced form compound (VI) (e.g. 424 nm) which, upon air oxidation, converts to the absorptions associated with the oxidized form of compound (VI) (e.g. 446 nm).

FIG. 24. UV-visible spectrum showing Mn(III)/Mn(II) ratio: sample was titrated with Mn(III) salt and tested for Mn incorporation at 0 min and 30 min.

FIG. 25. Mass spectrum for compound (VI) showing correctly identified mass.

FIG. 26. Titration curve and 1^{st} derivative plot of 75 mg/mL Formula (VI) with 1.0 N HCl: the solution was titrated with 1.0 N HCl at 30 μL increments.

FIG. 27. Chemical stability of 75 mg/mL Formula (VI) in water (pH 7) at 60° C.: air sparged samples provided better stability than the non-sparged sample; Soln-1A: Mixed solution for 24 hours at room temperature, open to air, before adjusting pH back to 6.8-7.2, then filtered through PVDF filter; Soln-1B: Control Solution—Mixed solution for 24 hours at room temperature, open to air, before adjusting pH back to 6.8-7.2, then filtered through PVDF filter. Soln-2A: Sparged compounding solution with air during mixing for about 4.5 hours then immediately adjusted pH to 6.8-7.2. Soln-2B: Sparged compounding solution with air during mixing for about 4.5 hours.

FIG. 28. pH stability of 75 mg/mL Formula (VI) in water (pH 7) at 60° C.: degradation from all samples stored at 60° C. was found to be 3-6% lower than that from the control sample after 14 days; Soln-1A: Mixed solution for 24 hours at room temperature, open to air, before adjusting pH back to 6.8-7.2, then filtered through PVDF filter; Soln-1B: Control Solution—Mixed solution for 24 hours at room temperature, open to air, before adjusting pH back to 6.8-7.2, then filtered through PVDF filter. Soln-2A: Sparged compounding solution with air during mixing for about 4.5 hours then immediately adjusted pH to 6.8-7.2. Soln-2B: Sparged compounding solution with air during mixing for about 4.5 hours.

FIG. 29. Chemical stability of 75 mg/mL Formula (VI) in water as a function of pH at 60° C.: pH shift of non-sparged sample (˜1 pH unit) was less than that of the sparged samples (˜1.5-2 pH units); Soln-1A: Mixed solution for 24 hours at room temperature, open to air, before adjusting pH back to 6.8-7.2, then filtered through PVDF filter; Soln-1B: Control Solution—Mixed solution for 24 hours at room temperature, open to air, before adjusting pH back to 6.8-7.2, then filtered through PVDF filter. Soln-2A: Sparged compounding solution with air during mixing for about 4.5 hours then immediately adjusted pH to 6.8-7.2. Soln-2B: Sparged compounding solution with air during mixing for about 4.5 hours.

FIG. 30. Chemical stability of various concentrations of Formula (VI) in water (pH 7) at 60° C.: the lower the pH, the greater the drug stability; Soln-1A: Mixed solution for 24 hours at room temperature, open to air, before adjusting pH back to 6.8-7.2, then filtered through PVDF filter; Soln-1B: Control Solution—Mixed solution for 24 hours at room temperature, open to air, before adjusting pH back to 6.8-7.2, then filtered through PVDF filter. Soln-2A: Sparged compounding solution with air during mixing for about 4.5 hours then immediately adjusted pH to 6.8-7.2. Soln-2B: Sparged compounding solution with air during mixing for about 4.5 hours.

FIG. 31. Chemical stability of various concentrations of Formula (VI) in water containing ascorbic acid (pH 7) at 60° C.

FIG. 32. pH stability of various concentrations of Formula (VI) in water (pH 7) at 60° C.: the samples were tested and evaluated for physicochemical stability under 2-8 and 60° C. storage conditions after 0, 3, 7 and 14 days—samples with and without ascorbic acid at 60° C. degraded relatively at the same rate ˜3-5% after 14 days.

FIG. 33. pH stability of various concentrations of Formula (VI) in water (pH 7) containing ascorbic acid after 14 day storage at 60° C.: the samples were tested and evaluated for physicochemical stability under 2-8 and 60° C. storage conditions after 0, 3, 7 and 14 days—samples with and without ascorbic acid at 60° C. degraded relatively at the same rate (˜3-5% after 14 days).

FIG. 34. Chemical stability of 75 mg/mL Formula (VI) in water (pH 7): No significant change of the sample was observed at each storage condition within an analytical variation after 1 month. HPLC purity assay of the pH 7 sample was observed to be dependent on temperature.

FIG. 35. pH stability of 75 mg/mL Formula (VI) in water (pH 7): refrigerated sample provided stability of pH 7 within 0.1 pH unit after 1 month, while the pH of samples at 25, 30 and 40° C. decreased approximately 0.3, 0.5 and 1.1 pH units, respectively (all samples provided the isotonic solution (270-276 mOsm/kg) without any significant change of) osmolality after 1 month.

FIG. 36. Chemical stability of 75 mg/mL Formula (VI) in water (pH 4, 5 and 6) after 14 days: the chemical stability of 75 mg/mL compound in water was evaluated at the pH range at 4-6 under the ICH storage temperatures i.e. 2-8, 25 and 40° C.—an accelerated 60° C. storage temperature was also accessed in order to compare and generate a pH-stability profile of drug in water—No significant changes of purity assays were observed after 14 days from the samples at pH between 4.1 and 6.8.

FIG. 37. pH stability of 75 mg/mL Formula (VI) in water after 14 day storage at 60° C.: increase of pH in such range yielded ˜5% decrease in drug purity assay; all other degradation products increased as a function of pH (e.g. a degradant at RRT 1.56-1.62 increased ˜8 folds (0.4-3.2%) within the pH profile range).

FIG. 38. pH stability of 75 mg/mL Formula (VI) in water at pH 4, 5 and 6: stability at pH 4 and 5 were well maintained after 14 days at all storage conditions within 0.1 pH unit variation—pH shifts were found in both directions at pH 6, where the changes were determined to be 0.7, 0.5, −0.1 and −0.9 pH units after 14 days under the storage conditions at 2-8, 25, 40, and 60° C., respectively.

FIG. 39: Crystal structure of compound (VI): The crystal was mounted with mineral oil (STP® Oil Treatment) on a MITEGEN™ mount; diffraction data (ψ- and ω-scans) were collected at 100K on a Bruker-AXS X8 Kappa Duo diffractometer coupled to a Smart Apex2 CCD detector with graphite-monochromated Mo Ka radiation (λ=0.71073 Å) from a fine-focus sealed tube.

FIGS. 40A-40B: Hydrogen bonding network of compound (VI): Carbon-bound hydrogen atoms omitted for clarity: FIG. 40A: Panel A shows the immediate surroundings of the target molecule (symmetry operator to generate atoms with a capital A at the end of their atom name: 1−x, 1−y, 1−z); FIG. 40B: Panel B shows the extended network.

FIG. 41: Crystal structure lattice of compound (VI): sheets extend parallel to the a-c-plane and are stacked along the b-direction, repeating twice per unit cell.

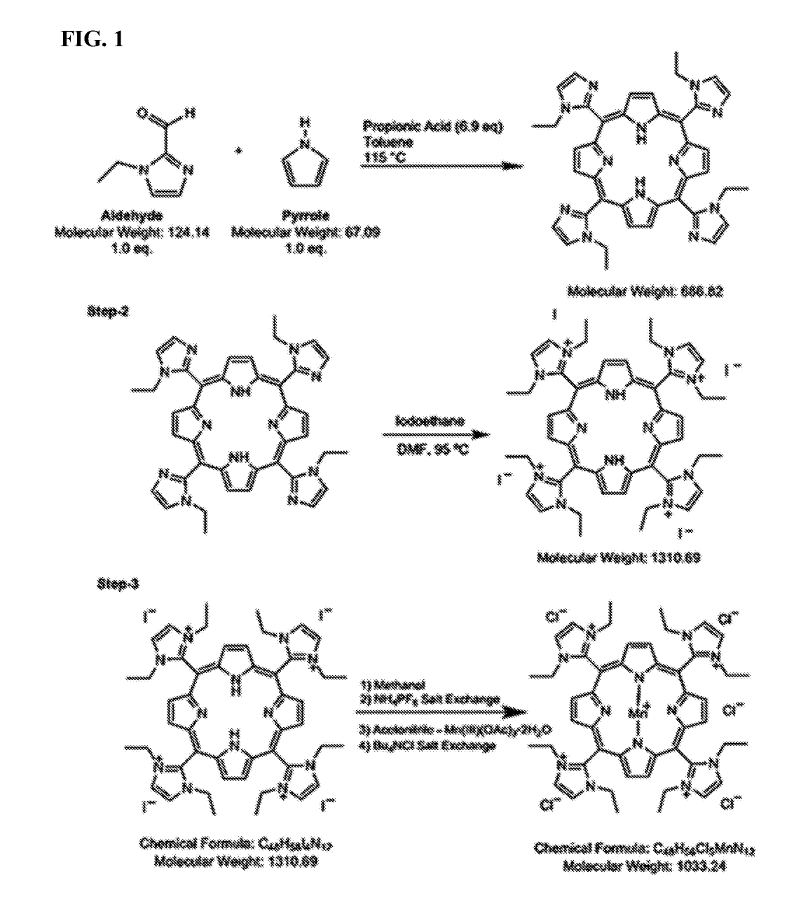

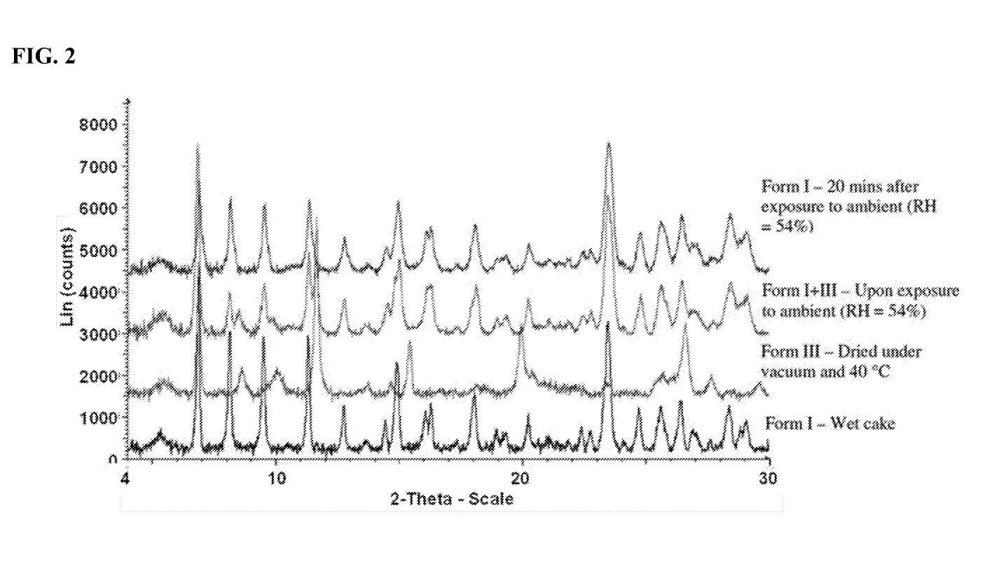

### DETAILED DESCRIPTION OF THE INVENTION

### I. DEFINITIONS

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Alkyl is not cyclized. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, S, Se and Si, and wherein the nitrogen, selenium, and sulfur atoms may optionally be oxidized, and the nitrogen heteroatom may optionally be quaternized. Heteroalkyl is not cyclized. The heteroatom(s) O, N, P, S, Se, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (e.g. 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom (e.g. N, O, or S), wherein sulfur heteroatoms are optionally oxidized, and the nitrogen heteroatoms are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The term “oxo,” as used herein, means an oxygen that is double bonded to a carbon atom.

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂N(R)(‘R″—NRSO₂R′), —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, NR″C(O)₂R′, NRC(NR′R″)═NR′″, S(O)R′, —S(O)₂R′, —S(O)₂N(R′)(R″, —NRSO₂R′), —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. The ring-forming substituents may be attached to adjacent members of the base structure. For example, two ring-forming substituents attached to adjacent members of a cyclic base structure create a fused ring structure. The ring-forming substituents may be attached to a single member of the base structure. For example, two ring-forming substituents attached to a single member of a cyclic base structure create a spirocyclic structure. The ring-forming substituents may be attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “ size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₃-C₈ aryl, and each substituted or unsubstituted heteroaryl is a substituted or unsubstituted C₃-C₈ heteroaryl.

Each substituted group described in the compounds herein may be substituted with at least one substituent group. More specifically, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein may be substituted with at least one substituent group.

Each substituted or unsubstituted alkyl may be a substituted or unsubstituted C₁-C₁₀ alkyl, each substituted or unsubstituted heteroalkyl may be a substituted or unsubstituted 2 to 20 membered heteroalkyl, each substituted or unsubstituted cycloalkyl may be a substituted or unsubstituted C₃-C₈ cycloalkyl, and/or each substituted or unsubstituted heterocycloalkyl may be a substituted or unsubstituted 3 to 8 membered heterocycloalkyl. Each substituted or unsubstituted alkylene may be a substituted or unsubstituted C₁-C₂₀ alkylene, each substituted or unsubstituted heteroalkylene may be a substituted or unsubstituted 2 to 20 membered heteroalkylene, each substituted or unsubstituted cycloalkylene may be a substituted or unsubstituted C₃-C₈ cycloalkylene, each substituted or unsubstituted heterocycloalkylene may be a substituted or unsubstituted 3 to 8 membered heterocycloalkylene, each substituted or unsubstituted arylene may be a substituted or unsubstituted C₃-C₈ arylene, and/or each substituted or unsubstituted heteroaryl may be a substituted or unsubstituted C₃-C₈ heteroarylene.

Each substituted or unsubstituted alkyl may be a substituted or unsubstituted C₁-C₈ alkyl, each substituted or unsubstituted heteroalkyl may be a substituted or unsubstituted 2 to 8 membered heteroalkyl, each substituted or unsubstituted cycloalkyl may be a substituted or unsubstituted C₃-C₇ cycloalkyl, each substituted or unsubstituted heterocycloalkyl may be a substituted or unsubstituted 3 to 7 membered heterocycloalkyl, each substituted or unsubstituted aryl may be a substituted or unsubstituted C₃-C₇ aryl, and/or each substituted or unsubstituted heteroaryl may be a substituted or unsubstituted C₃-C₇ heteroaryl. Each substituted or unsubstituted alkylene may be a substituted or unsubstituted C₁-C₈ alkylene, each substituted or unsubstituted heteroalkylene may be a substituted or unsubstituted 2 to 8 membered heteroalkylene, each substituted or unsubstituted cycloalkylene may be a substituted or unsubstituted C₃-C₇ cycloalkylene, each substituted or unsubstituted heterocycloalkylene may be a substituted or unsubstituted 3 to 7 membered heterocycloalkylene, each substituted or unsubstituted arylene may be a substituted or unsubstituted C₃-C₇ arylene, and/or each substituted or unsubstituted heteroarylene may be a substituted or unsubstituted C₃-C₇ heteroarylene.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those that are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

It should be noted that throughout the application that alternatives are written in Markush groups, for example, each ring position that contains more than one possible substituted moiety (e.g. pyridinyl, pyrazinyl, pyrimidinyl, or pyridazinyl). It is specifically contemplated that each member of the Markush group should be considered separately, thereby comprising another embodiment, and the Markush group is not to be read as a single unit.

The term “azeotrope” refers to a mixture of two or more solvents that has a constant boiling point. The components of an azeotrope cannot be separated via simple distillation. An azeotrope may be characterized as a positive azeotrope (e.g. a mixture having a lower boiling point than either of its components) or a negative azeotrope (e.g. a mixture having a higher boiling point than either of its components).

The terms “analog,” “analogue,” or “derivative” are used in accordance with their plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₁₀ alkyl, or unsubstituted 2 to 20 membered heteroalkyl,” the group may contain one or more unsubstituted C₁-C₁₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galacturonic acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Non-limiting examples of such salts include hydrochlorides, hydrobromides, phosphates (e.g. hexafluorophosphates), borates (e.g. tetrafluoroborates), thiocyanates, sulfates, nitrates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline, polymorphic, or amorphous forms. In general, all physical forms are equivalent for the uses contemplated by the present invention and are intended to be within the scope of the present invention.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, however, that the resulting reaction product can be produced directly from a reaction between the added reagents or from an intermediate formed from one or more of the added reagents.

The terms “Pharmaceutically acceptable excipient,” “pharmaceutical excipient” and “pharmaceutically acceptable carrier” are used interchangeably herein and refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethylcellulose, polyvinyl pyrrolidine, and colors, and the like. Pharmaceutical excipients as described herein do not include pH adjusting ions, such as, for example, ions derived from dissolution of acids or bases including but not limited to HCl or NaOH. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

### II. METHODS OF SYNTHESIS

In a first aspect a method is provided for synthesizing a substituted porphyrin having the formula:

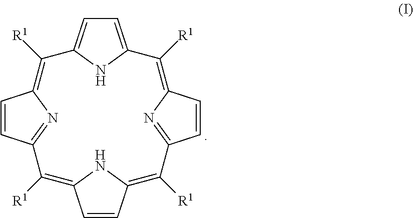

In formula (I), R¹ is substituted or unsubstituted heterocycloalkyl or substituted or unsubstituted heteroaryl. The method includes contacting a pyrrole with an R¹-substituted aldehyde. The contacting is performed in a solvent system which includes a positive azeotrope. The pyrrole is allowed to react with the R¹-substituted aldehyde in the solvent system under azeotropic distillation conditions, thereby forming a substituted-porphyrinogen. The substituted-porphyrinogen is oxidized, thereby synthesizing a substituted porphyrin having formula (I).

The contacting may be performed using about equal portions of pyrrole and the R¹-substituted aldehyde. The contacting may be performed using about one equivalent pyrrole and about one equivalent R¹-substituted aldehyde. R¹ may be substituted or unsubstituted heterocycloalkyl (e.g. 3 to 10 membered heterocycloalkyl). R¹ may be substituted or unsubstituted 3 to 10 membered heterocycloalkyl. R¹ may be substituted or unsubstituted 3 to 8 membered heterocycloalkyl. R¹ may be substituted or unsubstituted 4 to 6 membered heterocycloalkyl. R¹ may be substituted or unsubstituted 5 or 6 membered heterocycloalkyl. R¹ may be substituted or unsubstituted imidazolyl, substituted or unsubstituted pyrazolyl, substituted or unsubstituted thiazolyl, or substituted or unsubstituted triazolyl. R¹ may be unsubstituted imidazolyl, unsubstituted pyrazolyl, unsubstituted thiazolyl, or unsubstituted triazolyl. R1 may be substituted imidazolyl. R1 may be

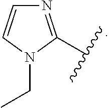 [SMILES: *c1nccn1CC]

R¹ may be substituted or unsubstituted imidazolium, substituted or unsubstituted pyrazolium, substituted or unsubstituted thiazolium, or substituted or unsubstituted triazolium. R¹ may be unsubstituted imidazolium, unsubstituted pyrazolium, unsubstituted thiazolium, or unsubstituted triazolium. R¹ may be substituted imidazolium.

R¹ may be R²-substituted or unsubstituted heterocycloalkyl (e.g. 3 to 10 membered heterocycloalkyl) or R²-substituted or unsubstituted heteroaryl (e.g. 5 to 8 membered heteroaryl). R¹ may be R²-substituted imidazolyl, R²-substituted pyrazolyl, R²-substituted thiazolyl, or R²-substituted triazolyl. R¹ may be R²-substituted imidazolium, R²-substituted pyrazolium, R²-substituted thiazolium, or R²-substituted triazolium. R² is independently hydrogen, halogen, —N₃, —CF₃, —CCl₃, —CBr₃, —CI₃, —CN, —CHO, —OH, —NH₂, —N(CH₃)₂, —COOH, —CONH₂, —NO₂, —SH, —SO₂Cl, —SO₃H, —SO₄H, —SO₂NH₂, —NHNH₂, —ONH₂, —NHC(O)NHNH₂, R³-substituted or unsubstituted alkyl (e.g. C₁ to C₈ alkyl), R³-substituted or unsubstituted heteroalkyl (e.g. 2 to 8 membered heteroalkyl), R³-substituted or unsubstituted cycloalkyl (e.g. C₃-C₈ cycloalkyl), R³-substituted or unsubstituted heterocycloalkyl (e.g. 3 to 6 membered heterocycloalkyl), R³-substituted or unsubstituted aryl (e.g. phenyl), or R³-substituted or unsubstituted heteroaryl (e.g. 5 or 6 membered heteroaryl).

R³ is independently hydrogen, halogen, —N₃, —CF₃, —CCl₃, —CBr₃, —CI₃, —CN, —CHO, —OH, —NH₂, —N(CH₃)₂, —COOH, —CONH₂, —NO₂, —SH, —SO₂Cl, —SO₃H, —SO₄H, —SO₂NH₂, —NHNH₂, —ONH₂, —NHC(O)NHNH₂, unsubstituted alkyl (e.g. C₁ to C₈ alkyl), unsubstituted heteroalkyl (e.g. 2 to 8 membered heteroalkyl), unsubstituted cycloalkyl (e.g. C₃-C₈ cycloalkyl), unsubstituted heterocycloalkyl (e.g. 3 to 6 membered heterocycloalkyl), unsubstituted aryl (e.g. phenyl), or unsubstituted heteroaryl (e.g. 5 or 6 membered heteroaryl). R¹ may be R²-substituted imidazolyl, wherein R² is C₁-C₃ unsubstituted alkyl. R² may be R³-substituted or unsubstituted alkyl (e.g. C₁ to C₈ alkyl). R² may be unsubstituted alkyl (e.g. C₁ to C₈ alkyl).

R¹ may be substituted or unsubstituted imidazolium. R¹ may be R²-substituted imidazolium, wherein R² is C₁-C₃ unsubstituted alkyl. R² may be ethyl. R¹ may be

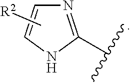

A person having ordinary skill in the art will immediately understand that R² may be attached to any atom of the imidazolium ring above having the appropriate valency.

R¹ may be substituted or unsubstituted heteroaryl (e.g. 5 to 8 membered heteroaryl). R¹ may be 5 to 8 membered substituted heteroaryl. R¹ may be 5 or 6 membered substituted heteroaryl. R¹ may be substituted or unsubstituted pyridinyl, substituted or unsubstituted pyrazinyl, substituted or unsubstituted pyrimidinyl, or substituted or unsubstituted pyridazinyl. R¹ may be unsubstituted pyridinyl, unsubstituted pyrazinyl, unsubstituted pyrimidinyl, or unsubstituted pyridazinyl. R¹ may be R²-substituted pyridinyl, R²-substituted pyrazinyl, R²-substituted pyrimidinyl, or R²-substituted pyridazinyl. R¹ may be substituted or unsubstituted pyridinium, substituted or unsubstituted pyrazinium, substituted or unsubstituted pyrimidinium, or substituted or unsubstituted pyridazinium. R¹ may be unsubstituted pyridinium, unsubstituted pyrazinium, unsubstituted pyrimidinium, or unsubstituted pyridazinium. R¹ may be R²-substituted pyridinium, R²-substituted pyrazinium, R²-substituted pyrimidinium, or R²-substituted pyridazinium. R² is as described herein, including embodiments thereof. R¹ may be

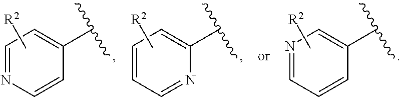

The contacting may be performed by rapid (e.g. less than 5 minutes) addition of the reagents (e.g. pyrrole and R¹-substituted aldehyde) or by slow addition of the reagents over a period of time. The addition may be performed from about 5 minutes to about 1 hour. When slow addition is performed, the addition may take place over about 1 hour to about 48 hours. The addition may be performed over about 1, 3, 6, 9, 10, 12, 15, 18, 21, 24, 27, 30, 33, 36, 39, 42, 45, or 48 hours. Slow addition may increase the yield of a compound of formula (I), including embodiments thereof.

The addition may be performed in an environment substantially free of air (e.g. under an atmosphere of nitrogen). The reaction may be performed under an atmosphere of nitrogen, argon, or other inert gas. The contacting may be performed in a low oxygen environment (e.g. oxygen concentrations less than about atmospheric oxygen concentrations). The oxygen concentration may be less than 25% of the gas contained in the reaction vessel. The oxygen concentration may be less than 20% of the gas contained in the reaction vessel. The oxygen concentration may be less than 15% of the gas contained in the reaction vessel. The oxygen concentration may be less than 10% of the gas contained in the reaction vessel. The oxygen concentration may be less than 5% of the gas contained in the reaction vessel. The oxygen concentration may be less than 1% of the gas contained in the reaction vessel. The addition may be performed in an environment exposed to air.

The contacting may be performed in a solvent system at a temperature of about 20 to about 120° C. The contacting may be performed in a solvent system at a temperature of about 20, 25, 30, 35, 40, 45, 50, 55, 60, 65, 70, 75, 80, 85, 90, 95, 100, 105, 110, 115 or 120° C. The contacting may be performed in a solvent system at a temperature of about 75° C. The contacting may be performed in a solvent system at a temperature of about 80° C. The contacting may be performed in a solvent system at a temperature of about 90° C. The contacting may be performed in a solvent system at a temperature of about 100° C. The contacting may be performed in a solvent system at a temperature of about 105° C. The contacting may be performed in a solvent system at a temperature of about 110° C. The contacting may be performed in a solvent system at a temperature of about 115° C. The contacting may be performed in a solvent system at a temperature of about 120° C.

The oxidizing may be performed by exposure to air or by using an oxidant. The oxidizing may be performed by exposing the reaction mixture to air. The oxidizing may be performed using an oxidant. The oxidant may be 2,3-dichloro-5,6-dicyano-1,4-benzoquinone. The oxidizing may be performed in a low oxygen environment as described herein. The oxidizing may be performed in the absence of an exogenous oxidant (i.e. the reaction supplies the oxidant). The oxidizing may be performed in a low oxygen environment as described herein and in the absence of an exogenous oxidant.

The solvent system may include a first solvent and an acid. The first solvent may be chlorobenzene, m-xylene, or toluene. The first solvent may be chlorobenzene. The first solvent may be m-xylene. The first solvent may be toluene. The acid may be a carboxylic acid. The carboxylic acid may be acetic acid, formic acid, propionic acid, valeric acid, or butyric acid. The carboxylic acid may be acetic acid. The carboxylic acid may be formic acid. The carboxylic acid may be propionic acid. The carboxylic acid may be valeric acid. The carboxylic acid may be butyric acid.

Positive azeotropes are typically selected based on appropriate boiling temperatures and their ability to solubilize the chemical reactants and or products. The azeotrope may have a boiling temperature greater than water (e.g. 100° C.) to allow for removal of water during the reacting (e.g. azeotropic distillation). The azeotrope may have a boiling temperature less than water (e.g. 100° C.) to allow for removal of water during the reacting (e.g. azeotropic distillation). The positive azeotrope may be formed during the reaction (e.g. water formed during a condensation reaction may be removed using an azeotrope formed by the water produced and a solvent of the reaction). The positive azeotrope may include an acid (e.g. a carboxylic acid described herein) and a first solvent as described herein. The first solvent may be an organic solvent, such as toluene. The positive azeotrope may be formed by a mixture of propionic acid and toluene.

The pyrrole may react with the R¹-substituted aldehyde in the solvent under azeotropic distillation conditions (e.g. distillation using an azeotropic mixture to dehydrate the reaction), thereby forming a substituted-porphyrinogen. When reacted under azeotropic distillation conditions, water may be removed from the reaction.

The methods disclosed herein may provide yields of a compound of formula (I), including embodiments thereof, from about 6% to about 35%. The yield may be from about 8% to about 35%. The yield may be from about 10% to about 35%. The yield may be from about 15% to about 35%. The yield may be from about 6% to about 30%. The yield may be from about 8% to about 30%. The yield may be from about 10% to about 30%. The yield may be from about 15% to about 30%. The yield may be from about 6% to about 25%. The yield may be from about 8% to about 25%. The yield may be from about 10% to about 25%. The yield may be from about 15% to about 25%. The yield may be from about 6% to about 20%. The yield may be from about 8% to about 20%. The yield may be from about 10% to about 20%. The yield may be from about 6% to about 15%. The yield may be from about 8% to about 15%. The yield may be from about 10% to about 15%. The yield may be from about 6% to about 10%. The yield may be from about 8% to about 10%.

The methods disclosed herein may provide yields of the substituted porphyrin of formula (I) in at least about 6%. The yield may be at least about 8%. The yield may be at least about 10%. The yield may be at least about 15%. The yield may be at least about 20%. The yield may be at least about 25%. The yield may be at least about 30%. The substituted porphyrin may be isolated in an environment substantially free of air (e.g. under a nitrogen blanket) as described herein.

The reacting of pyrrole with the R¹-substituted aldehyde may be performed at a temperature from about 40° C. to about 150° C. The reacting may be performed at a temperature of above 40, 45, 50, 55, 60, 65, 70, 75, 80, 85, 90, 95, 100, 105, 110, 115, 120, 125, 130, 135, 140 or about 150° C. The reacting may be performed at a temperature of about 140° C. The reacting may be performed at a temperature of about 120° C. The reacting may performed over a period of time from about 1 hour to about 16 hours. The reacting may performed over a period of time of about 1, 2, 3, 4, 5, 6, 7, 8, 9, 10, 11, 12, 13, 14, 15, or 16 hours. The reacting may performed over a period of time of about 1 hour. The reacting may performed over a period of time of about 2 hours. The reacting may performed over a period of time of about 3 hours. The reacting may performed over a period of time of about 4 hours. The reacting may performed over a period of time of about 5 hours. The reacting may performed over a period of time of about 6 hours. The reacting may performed over a period of time of about 7 hours. The reacting may performed over a period of time of about 8 hours. The reacting may performed over a period of time of about 9 hours. The reacting may performed over a period of time of about 10 hours. The reacting may performed over a period of time of about 11 hours. The reacting may performed over a period of time of about 12 hours. The reacting may performed over a period of time of about 13 hours. The reacting may performed over a period of time of about 14 hours. The reacting may performed over a period of time of about 15 hours. The reacting may performed over a period of time of about 16 hours. The method may further include removing the solvent after the reaction. The method may include filtering the solvent after the reaction. The method may include purifying the compound of formula (I) using techniques and methods described herein, including embodiments thereof. The compound of formula (I) may be purified from methyl-ethyl-ketone (2-butanone or MEK) or dimethylformamide (DMF).

The pyrrole and the R¹-substituted aldehyde may be contacted in a reaction vessel in a single addition of each reagent. The pyrrole and the R¹-substituted aldehyde may be contacted in a reaction vessel in at least two portions (i.e. 2 separate additions of each reagent). The pyrrole and the R¹-substituted aldehyde may be contacted in a reaction vessel in at least three portions (i.e. 3 separate additions of each reagent). The pyrrole and the R¹-substituted aldehyde may be contacted in a reaction vessel in at least four portions (i.e. 4 separate additions of each reagent). The pyrrole and the R¹-substituted aldehyde may be contacted in a reaction vessel in at least five portions (i.e. 5 separate additions of each reagent).The pyrrole and the R¹-substituted aldehyde may be contacted in a reaction vessel in at least six portions (i.e. 6 separate additions of each reagent). The pyrrole and the R¹-substituted aldehyde may be contacted in a reaction vessel in at least seven portions (i.e. 7 separate additions of each reagent). The pyrrole and the R¹-substituted aldehyde may be contacted in a reaction vessel in at least eight portions (i.e. 8 separate additions of each reagent). The pyrrole and the R¹-substituted aldehyde may be contacted in a reaction vessel in at least nine portions (i.e. 9 separate additions of each reagent). The pyrrole and the R¹-substituted aldehyde may be contacted in a reaction vessel in at least ten portions (i.e. 10 separate additions of each reagent). When the pyrrole and R¹-substituted aldehyde are added in portions, the portions may be of equal concentration.

The reacting of the pyrrole with the R¹-substituted aldehyde forms a reduced substituted-porphyrinogen intermediate. The reduced substituted-porphyrinogen intermediate may be oxidized to formula (I) by exposure to air or by using an oxidant. When oxidation is performed using an oxidant (e.g. exogenous oxidant), the oxidant may be 2,3-Dichloro-5,6-dicyano-1,4-benzoquinone (DDQ), m-chloroperoxybenzoic acid (m-CPBA), p-chloranil, or iron-pthalocyanine. Oxidation of the reduced substituted-porphyrinogen intermediate may occur in-situ. Oxidation of the reduced substituted-porphyrinogen may occur in the absence of exogenous oxidant (i.e. the reaction supplies the oxidant). The oxidizing may be performed in a low oxygen environment as described herein. The oxidizing may be performed in a low oxygen environment as described herein and in the absence of an exogenous oxidant.

The compound of formula (I), including embodiments thereof, may have formula:

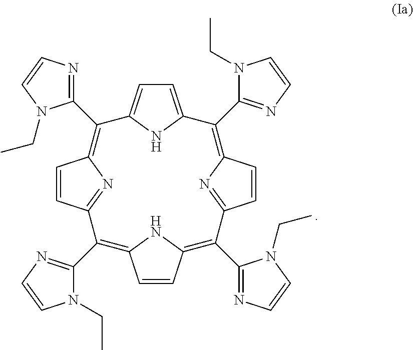 [SMILES: CCn1ccnc1-c1c2nc(c(-c3nccn3CC)c3ccc([nH]3)c(-c3nccn3CC)c3nc(c(-c4nccn4CC)c4ccc1[nH]4)C=C3)C=C2]

The method may further include contacting the compound of formula (I), including embodiments thereof, or formula (Ia), including embodiments thereof, with a metal salt. The metal salt may a transition metal salt (e.g. those elements in Periods 4 through 7 of the periodic table). More specifically, the transition metal may be a manganese (Mn) salt. The Mn salt may be a Mn(II) or Mn(III) salt, such as, for example, Mn(III) acetate or Mn(III) chloride. The compound may be recrystallized as described herein.

The method may further include contacting the compound of formula (Ia) with a volume of water and stirring the mixture for a period of time (e.g. 0.5, 1, 1.5, 2, 2.5, or 3 hours). The addition of water may remove residual excess sodium propionate formed during the reaction.

In another aspect, is a method for synthesizing a compound of formula:

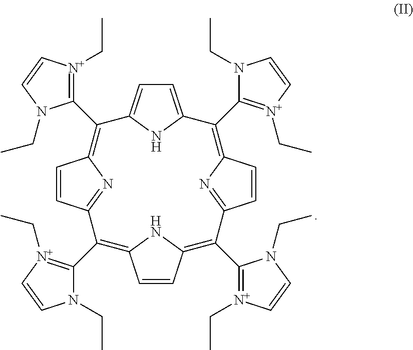 [SMILES: CCn1cc[n+](CC)c1-c1c2nc(c(-c3n(CC)cc[n+]3CC)c3ccc([nH]3)c(-c3n(CC)cc[n+]3CC)c3nc(c(-c4n(CC)cc[n+]4CC)c4ccc1[nH]4)C=C3)C=C2]

The method includes contacting with an ethylating agent a compound having the formula

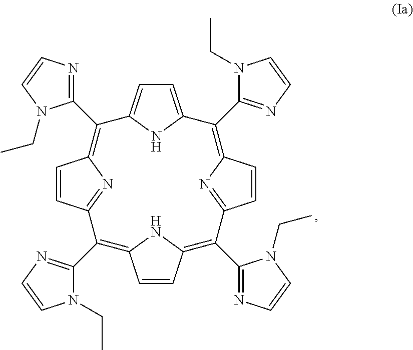 [SMILES: CCn1ccnc1-c1c2nc(c(-c3nccn3CC)c3ccc([nH]3)c(-c3nccn3CC)c3nc(c(-c4nccn4CC)c4ccc1[nH]4)C=C3)C=C2]

thereby synthesizing a compound of formula (II).

Formula (Ia), including embodiments thereof, may include a counterion. The counterion may be selected from the group consisting of a halogen anion, SCN⁻, SO₄⁻², HSO₄⁻, H₂PO₄⁻, HPO₄⁻², PO₄⁻³, NO₃⁻, PF₆⁻, or BF₄⁻. When the counterion is halogen the anion may be F⁻, Cl⁻, Br⁻, or I⁻. The counterion may be Cl⁻. One skilled in art would recognize that any appropriate counterion could be present, including those that are pharmaceutically acceptable such as those described herein.

The method may further include contacting about equal portions of pyrrole and 1-ethyl-1H-imidazole-2-carbaldehyde as described herein. The contacting may be performed in a solvent system that includes a positive azeotrope, as described herein, including embodiments thereof. The method may include contacting about one equivalent of a pyrrole with about one equivalent of 1-ethyl-1H-imidazole-2-carbaldehyde. The pyrrole may react with the 1-ethyl-1H-imidazole-2-carbaldehyde, in the solvent system under azeotropic distillation conditions, as described herein, including embodiments thereof, thereby forming a substituted-porphyrinogen. The substituted-porphyrinogen may be oxidized, thereby synthesizing a substituted porphyrin having formula (Ia).

The ethylating agent may be an alkyl-halogen. The alkyl-halogen may be a C₁-C₃ unsubstituted alkyl-halogen. The alkyl-halogen may be iodoethane. The ethylating agent may be present in excess compared to the compound of formula (Ia). About 1, 2, 3, 4, 5, 10, 15, 20, 25, 30, 35, 40, 45, 50, or 55 equivalents of the ethylating agent may be contacted with the compound of formula (Ia). The ethylating agent may be added at about 33 equivalents compared to the compound of formula (Ia). The ethylating agent may be added at about 40 equivalents compared to the compound of formula (Ia). The ethylating agent may be added at about 43 equivalents compared to the compound of formula (Ia). The ethylating agent may be added at about 53 equivalents compared to the compound of formula (Ia).

The reaction may be performed in dimethylformamide, ethyl acetate, or a mixture of dimethylformamide and ethyl acetate. When performed in a mixture, the volume of ethyl acetate may be greater than the volume of dimethylformamide. The volume of ethyl acetate may be about 1.5×, 2.0×, 2.5×, 3.0×, 3.5×, or 4.0× greater than the volume of dimethylformamide. The volume of ethyl acetate may be about 1.7× greater than the volume of dimethylformamide. The volume of ethyl acetate may be about 2.7× greater than the volume of dimethylformamide. The volume of ethyl acetate may be about 3.7× greater than the volume of dimethylformamide.

The contacting may be performed at a temperature from about 20° C. to about 120° C. The contacting performed at a temperature from about 50° C. to about 100° C. The contacting may be performed at a temperature of about 20, 25, 30, 35, 40, 45, 50, 55, 60, 65, 70, 75, 80, 85, 90, 95, 100, 105, 110, 115, or about 120° C. The contacting may be performed at a temperature of about 50° C. The contacting may be performed at a temperature of about 80° C. The contacting may be performed at a temperature of about 85° C. The contacting may be performed at a temperature of about 95° C. The contacting may be performed at a temperature of about 105° C.

The method may further include precipitating the compound of formula (Ia), including embodiments thereof, by adding an ammonium salt, such as for example, ammonium hexafluorophosphate. The ammonium salt may be pre-dissolved in an organic solvent, such as, for example, methanol, ethanol, or acetonitrile. The method may include anion exchange, wherein the counterions described herein are exchanged with a halogen anion such as, for example, Cl⁻, or PF₆⁻. Ion exchange may occur upon precipitation with an ammonium salt (e.g. ammonium hexafluorophosphate). One skilled in art would recognize that any appropriate counterion could be present including those that are pharmaceutically acceptable such as those described herein.

The ethylating agent may be a Meerwein salt. The Meerwein salt may be trialkyloxonium tetrafluoroborate or trialkyloxonium hexafluorophosphate. The alkyl group may be unsubstituted methyl or unsubstituted ethyl. The Meerwein salt can be a trimethyloxonium tetrafluoroborate, a triethyloxonium tetrafluoroborate, trimethyloxonium hexafluorophosphate, or a triethyloxonium hexafluorophosphate. The Meerwein salt can be a trimethyloxonium tetrafluoroborate. The Meerwein salt can be a triethyloxonium tetrafluoroborate. The Meerwein salt can be a trimethyloxonium hexafluorophosphate. The Meerwein salt can be a triethyloxonium hexafluorophosphate. The contacting may be performed in an organic solvent, such as, for example, dimethylformamide (DMF), acetonitrile (MeCN), dichloromethane (DCM), or tert-butyl methyl ether (tBME). The contacting may be performed in dimethylformamide or acetonitrile. The contacting may be performed in an acetonitrile solvent. The contacting may be performed in dimethylformamide. The contacting may be performed at a temperature as described herein, including embodiments thereof.

The method may include precipitation of the compound having formula (II), including embodiments thereof, with a precipitating agent. The precipitating agent may be an ammonium salt, such as, for example, tetrabutyl ammonium chloride (Bu₄NCl) or ammonium hexafluorophosphate (NH₄PF₆). The precipitating agent may be tetrabutyl ammonium chloride (Bu₄NCl). The precipitating agent may exchange the counterions with Cl⁻ or PF₆⁻. The precipitating agent may be dissolved in acetonitrile or methanol. Thus, in embodiments, the precipitation may be performed using tetrabutyl ammonium chloride (Bu₄NCl) in acetonitrile. The compound having formula (II), including embodiments thereof, may be triturated with methanol containing an ammonium salt (e.g. ammonium hexafluorophosphate) at about 20° C. or about 60° C. The compound having formula (II), including embodiments thereof, may be triturated with a mixture of dichloromethane/acetone (2:1) containing an ammonium salt (e.g. ammonium hexafluorophosphate). The compound having formula (II), including embodiments thereof, may be triturated with water containing an ammonium salt (e.g. ammonium hexafluorophosphate). The compound having formula (II), including embodiments thereof, may be re-precipitated from acetone with methanol or ethyl acetate containing an ammonium salt (e.g. ammonium hexafluorophosphate). The compound having formula (II), including embodiments thereof, may be re-precipitated from dimethylformamide with ethyl acetate containing an ammonium salt (e.g. ammonium hexafluorophosphate). The purity of the precipitated or triturated compound having formula (II), including embodiments thereof, may be at least about 85, 86, 87, 88, 89, 90, 91, 92, 93, 94, 95, 96, 97, 98, 99, or 100%. The purity may be about 90 to about 100%. The purity may be at least 90%. The purity may be at least 91%. The purity may be at least 92%. The purity may be at least 93%. The purity may be at least 94%. The purity may be at least 95%. The purity may be at least 96%. The purity may be at least 97%. The purity may be at least 98%. The purity may be at least 99%.

The precipitation may be done at a temperature of about 10° C. to about 50° C. The precipitation may be done at a temperature of about 10° C. to about 40° C. The precipitation may be done at a temperature of about 10° C. to about 30° C. The precipitation may be done at a temperature of about 10° C. to about 25° C. The precipitation may be done at a temperature of about 10° C. The precipitation may be done at a temperature of about 15° C. The precipitation may be done at a temperature of about 20° C. The precipitation may be done at a temperature of about 21° C. The precipitation may be done at a temperature of about 22° C. The precipitation may be done at a temperature of about 23° C. The precipitation may be done at a temperature of about 24° C. The precipitation may be done at a temperature of about 25° C. The precipitation may be done at a room temperature (e.g. about 23° C.).

The method may include contacting the compound of formula (II), including embodiments thereof, with a metal salt as described herein. The metal salt may a transition metal salt (e.g. those elements in Periods 4 through 7 of the periodic table). More specifically, the transition metal may be a manganese (Mn) salt, as described herein. The Mn salt may be a Mn(II) or Mn(III) salt, such as, for example, Mn(III) acetate or Mn(III) chloride. Excess Mn(III) may reoxidize Mn(II) to Mn(III), thereby increasing the yield of a compound having formula (II) when contacted with a manganese salt.

In another aspect, is a method for synthesizing a hydrate compound having the formula

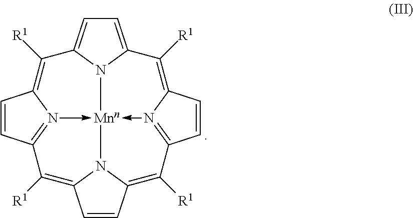

R¹ of formula (III) is as described hereinabove for compounds of formula (I). The symbol n is 2 or 3. The method includes contacting a compound of formula (I) with over about 2 equivalents of a Mn(III) salt in a solvent, thereby forming a reaction mixture. The reaction mixture is heated thereby synthesizing a compound of formula (III). The compound of formula (III) is hydrated thereby forming a hydrate of compound (III). The symbol n represents the oxidation state of the Mn (e.g. where n is 2, the Mn is in a Mn(II) oxidation state and where n is 3, the Mn is in a Mn(III) oxidation state).

R¹ is as described herein, including embodiments thereof. R¹ may be

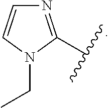 [SMILES: *c1nccn1CC]

The symbol n may be 3 (e.g. Mn(III)). The compound of formula (I), including embodiments thereof, may be contacted with more than about 1.2 equivalents to about 10 equivalents of a Mn(III) salt. The compound of formula (I), including embodiments thereof, may be contacted with about 2 equivalents to about 10 equivalents of a Mn(III) salt. The compound of formula (I), including embodiments thereof, may be contacted with over about 1.2 equivalents to about 5 equivalents of a Mn(III) salt. The compound of formula (I) including embodiments thereof, may be contacted with about 2 to about 5 equivalents of a Mn(III) salt. The compound of formula (I), including embodiments thereof, may be contacted with more than about 1.2 equivalents to about 3 equivalents of a Mn(III) salt. The compound of formula (I), may be contacted with about 2 to about 3 equivalents of a Mn(III) salt. The compound of formula (I), including embodiments thereof, may be contacted with more than about 1.2 equivalents of a Mn(III) salt. The compound of formula (I), including embodiments thereof, may be contacted with more than about 1.5 equivalents of a Mn(III) salt. The compound of formula (I), including embodiments thereof, may be contacted with about 2 equivalents of a Mn(III) salt. The compound of formula (I), including embodiments thereof, may be contacted with more than about 2.5 equivalents of a Mn(III) salt. The compound of formula (I), including embodiments thereof, may be contacted with about 3 equivalents of a Mn(III) salt. The compound of formula (I), including embodiments thereof, may be contacted with about 5 equivalents of a Mn(III) salt. The compound of formula (I), including embodiments thereof, may be contacted with about 10 equivalents of a Mn(III) salt. The number of equivalents used may maximize oxidation of the Mn to the Mn(III) oxidation state. The Mn(III) salt may be Mn(III) acetate. The Mn(III) salt may be Mn(III) chloride.

The method may be performed using dimethylformamide or acetonitrile as the solvent. The solvent may be a non-aqueous solvent. The solvent may be acetonitrile. The solvent may include a percent water content (e.g. v/v). The water content of the solvent may be about 0.5% to about 5%. The water content of the solvent may be about 1% to about 5%. The water content of the solvent may be about 1% to about 4%. The water content of the solvent may be about 1% to about 3%. The water content of the solvent may be about 1% to about 2%. The water content of the solvent may be about 2% to about 5%. The water content of the solvent may be about 2% to about 4%. The water content of the solvent may be about 2% to about 3%. The water content of the solvent may be about 1%. The water content of the solvent may be about 2%. The water content of the solvent may be about 3%.

The method may include contacting the reaction mixture with an anion-exchanging agent and allowing the reaction mixture to react with the anion-exchanging agent. The anion exchange may be performed as described herein, including embodiments thereof. The counterion may be exchanged to a Cl⁻ or a PF₆⁻ counterion, as described herein. One skilled in art would recognize that any appropriate counterion could be present, including those that are pharmaceutically acceptable such as those described herein. The counterion may be exchanged during a precipitation step with an ammonium salt, as described herein. The ammonium salt may be Bu₄NCl or NH₄PF₆.

The reaction mixture may be heated to a temperature of about 15° C. to about 70° C. The reaction mixture may be heated to a temperature of about 15, 20, 25, 30, 35, 40, 45, 50, 55, 60, 65, or 70° C. The reaction mixture may be heated to a temperature of about 15° C. The reaction mixture may be heated to a temperature of about 20° C. The reaction mixture may be heated to a temperature of about 23° C. (e.g. room temperature). The reaction mixture may be heated to a temperature of about 30° C. The reaction mixture may be heated to a temperature of about 40° C. The reaction mixture may be heated to a temperature of about 50° C. The reaction mixture may be heated to a temperature of about 65° C. The reaction may be heated for about 2 to about 80 hours. The reaction may be heated for about 4 to about 80 hours. The reaction may be heated for about 4 to about 50 hours. The reaction may be heated for about 10 to about 50 hours. The reaction may be heated to completion and allowed to react for an additional time thereafter (e.g. 2, 4, 6, or 8 hours). The method may further include filtering the reaction mixture. The filtering of the reaction mixture may occur before or after the heating.

The method may include allowing the reaction mixture to cool to a temperature of about 5° C. to about 50° C. The method may include allowing the reaction to cool to a temperature of about 10° C. to about 30° C. The cooling may occur rapidly or over a specific time period (e.g. about 1 hour to about 24 hours).

The method may further include precipitating the compound of formula (III), including embodiments thereof. The precipitation may be performed using an ammonium salt, as described herein. The ammonium salt may be tetrabutyl ammonium chloride (Bu₄NCl) or ammonium hexafluorophosphate (NH₄PF₆). The precipitating agent may be tetrabutyl ammonium chloride (Bu₄NCl). The precipitating agent may exchange the counterions with Cl⁻ or PF₆⁻. The precipitating agent may be dissolved in acetonitrile or methanol. Thus, in embodiments, the precipitation may be performed using tetrabutyl ammonium chloride (Bu₄NCl) in acetonitrile.

The precipitation may be done at a temperature of about 10° C. to about 50° C. The precipitation may be done at a temperature of about 10° C. to about 40° C. The precipitation may be done at a temperature of about 10° C. to about 30° C. The precipitation may be done at a temperature of about 10° C. to about 25° C. The precipitation may be done at a temperature of about 10° C. The precipitation may be done at a temperature of about 15° C. The precipitation may be done at a temperature of about 20° C. The precipitation may be done at a temperature of about 21° C. The precipitation may be done at a temperature of about 22° C. The precipitation may be done at a temperature of about 23° C. The precipitation may be done at a temperature of about 24° C. The precipitation may be done at a temperature of about 25° C. The precipitation may be done at a room temperature (e.g. about 23° C.).

Hydrating the compound of formula (III), including embodiments thereof, may include contacting a compound of formula (III), including embodiments thereof, with a gas having a relative humidity (“RH”) from about 10% to about 90% (i.e. passing a gas having a predetermined % water vapor (RH) through or over the compound). The gas having a RH may be saturated with water vapor (i.e. the gas contains water vapor at the highest percentage possible before precipitation of the vapor into liquid H₂O). The hydration may include contacting a compound of formula (III), including embodiments thereof, with a gas having a RH from about 20% to about 80%. The hydration may include contacting a compound of formula (III), including embodiments thereof, with a gas having a RH from about 50% to about 90%. The hydration may include contacting a compound of formula (III), including embodiments thereof, with a gas having a RH from about 60% to about 80%. The hydration may include contacting a compound of formula (III), including embodiments thereof, with a gas having a RH of about 68%. The hydration may include contacting a compound of formula (III), including embodiments thereof, with a gas having a RH from about 40% to about 60%. The hydration may include contacting a compound of formula (III), including embodiments thereof, with a gas having a RH described herein from about 30% to about 70%. The gas having a RH described herein may be an inert gas, such as for example, nitrogen or argon.

The compound of formula (III), including embodiments thereof, may be dried by contacting with a gas having a RH described herein. The drying may be performed by passing nitrogen or argon having a RH described herein over the compound for a period of time (e.g. about 16 to about 24 hours). When using a gas having a RH described herein to dry the compounds described herein, the water content in the drying sample (e.g. hydrated compound) may remain about the same (i.e. little to no change in the water content of the hydrated compound). The drying may be performed under vacuum.

The temperature of the gas having a RH described herein may be about 10° C. to about 40° C. The temperature of the gas having a RH described herein may be about 10° C. to about 40° C. The temperature of the gas having a RH described herein may be about 10° C. to about 35° C. The temperature of the gas having a RH described herein may be about 10° C. to about 30° C. The temperature of the gas having a RH described herein may be about 10° C. to about 25° C. The temperature of the gas having a RH described herein may be about 10° C. to about 15° C. The temperature of the gas having a RH described herein may be about 15° C. to about 40° C. The temperature of the gas having a RH described herein may be about 15° C. to about 35° C. The temperature of the gas having a RH described herein may be about 15° C. to about 30° C. The temperature of the gas having a RH described herein may be about 15° C. to about 25° C. The temperature of the gas having a RH described herein may be about 15° C. to about 20° C. The temperature of the gas having a RH described herein may be about 10° C. The temperature of the gas having a RH described herein may be about 11° C. The temperature of the gas having a RH described herein may be about 12° C. The temperature of the gas having a RH described herein may be about 13° C. The temperature of the gas having a RH described herein may be about 14° C. The temperature of the gas having a RH described herein may be about 15° C. The temperature of the gas having a RH described herein may be about 16° C. The temperature of the gas having a RH described herein may be about 17° C. The temperature of the gas having a RH described herein may be about 18° C. The temperature of the gas having a RH described herein may be about 19° C. The temperature of the gas having a RH described herein may be about 20° C. The temperature of the gas having a RH described herein may be about 25° C. The temperature of the gas having a RH described herein may be about 30° C. The temperature of the gas having a RH described herein may be about 35° C. The temperature of the gas having a RH described herein may be about 40° C.

Hydrating the compound of formula (III), including embodiments thereof, may occur in-situ in the presence of an aqueous solvent. The aqueous solvent may be a mixture of water and an organic solvent such as, for example, isopropanol, methanol, dimethylformamide, acetonitrile, or mixtures thereof. The mixture may contain about 0.5 to about 20% water as described herein. In-situ hydration of formula (III), including embodiments thereof, may replace residual solvent molecules from prior synthetic steps with water molecules.

The compound of formula (III) may have the formula:

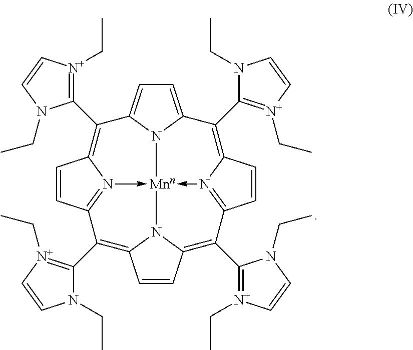

The compound of formula (IV), including embodiments thereof, may include a counterion selected from the group consisting of a halogen anion, SCN⁻, SO₄⁻², HSO₄⁻, H₂PO₄⁻, HPO₄⁻², PO₄⁻³, NO₃⁻, PF₆⁻, or BF₄⁻. The halogen anion may be F, Cl, Br, or I. The counterion may be Cl⁻. One skilled in art would recognize that any appropriate counterion could be present including those that are pharmaceutically acceptable such as those described herein. The counterion may be exchanged during a precipitation step with an ammonium salt, as described herein. The ammonium salt may be Bu₄NCl or NH₄PF₆.

The symbol n is as described herein, including embodiments thereof. The symbol n may be 3 (e.g. Mn(III)).

In another aspect is a method for purifying a compound of formula.

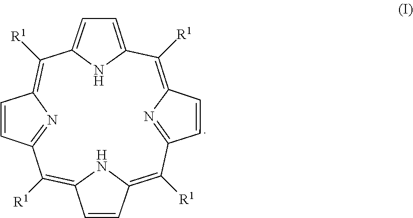

The method includes combining a compound of formula (I) and a purification solvent in a reaction vessel thereby forming a purification mixture. The compound is insoluble in the purification solvent. The purification mixture is heated. The purification mixture is cooled. The purification mixture is filtered, thereby purifying a compound of formula (I). The purification mixture may be cooled after the purification mixture is heated.

The purification solvent may be a solvent listed in Table 1.1. The purification solvent may be 2-butanone, 1,4-dioxane, acetonitrile, ethyl acetate or cyclohexanone. The purification solvent may be 2-butanone. The purification solvent may be 1,4-dioxane. The purification solvent may be acetonitrile. The purification solvent may be ethyl acetate. The purification solvent may be cyclohexanone. The percent recovery may be at least 30%. The percent recovery may be at least 40%. The percent recovery may be at least 50%. The percent recovery may be at least 60%. The percent recovery may be at least 70 The percent recovery may be at least 80%. The percent recovery may be at least 90%. The percent recovery may be at least 91%. The percent recovery may be at least 92%. The percent recovery may be at least 93%. The percent recovery may be at least 94%. The percent recovery may be at least 95%. The percent recovery may be at least 96%. The percent recovery may be at least 97%. The percent recovery may be at least 98%. The percent recovery may be at least 99%.

TABLE 1.1
Listing of purification solvents
Purification Solvent

| | |
|---|---|
| MEK (Run 1) | IPA/Heptane 1:1 |
| 1,4-dioxane | Toluene/DCM 1:1 |
| Ethyl acetate | Isopropyl acetate |
| Acetonitrile | Methyl-THF |
| 3-Pentanone | MIBK |
| 2-Pentanone | Isopentyl acetate |
| TBME/DCM 1:1 | Cyclohexanone |

The purification mixture may be heated to about 60° C. to about 100° C. The purification mixture may be heated to about 60° C. to about 90° C. The purification mixture may be heated to about 60° C. to about 80° C. The purification mixture may be heated to about 60° C. to about 70° C. The purification mixture may be heated to about 70° C. to about 90° C. The purification mixture may be heated to about 70° C. to about 85° C. The purification mixture may be heated to about 60° C. to about 70° C. The purification mixture may be heated to about 70° C. to about 80° C. The purification mixture may be heated to about 80° C. to about 90° C. The purification mixture may be heated to about 80° C. to about 85° C. The purification mixture may be heated to about 60° C. The purification mixture may be heated to about 70° C. The purification mixture may be heated to about 75° C. The purification mixture may be heated to about 80° C. The purification mixture may be heated to about 85° C. The purification mixture may be heated to about 90° C. The purification mixture may be heated to about 95° C. The purification mixture may be heated to about 100° C.

The purification mixture may be heated for at least 20 min. The purification mixture may be heated for at least 20 min. The purification mixture may be heated for at least 30 min. The purification mixture may be heated for at least 40 min. The purification mixture may be heated for at least 50 min. The purification mixture may be heated for at least 60 min. The purification mixture may be heated for at least 70 min. The purification mixture may be heated for at least 80 min. The purification mixture may be heated for at least 90 min. The purification mixture may be heated for at least 100 min. The purification mixture may be heated for at least 110 min. The purification mixture may be heated for at least 120 min. The purification mixture may be heated for about 20 min. The purification mixture may be heated for about 30 min. The purification mixture may be heated for about 40 min. The purification mixture may be heated for about 50 min. The purification mixture may be heated for about 1 hour. The purification mixture may be heated for about 1.1 hours. The purification mixture may be heated for about 1.2 hours. The purification mixture may be heated for about 1.3 hours. The purification mixture may be heated for about 1.4 hours. The purification mixture may be heated for about 1.5 hours. The purification mixture may be heated for about 1.6 hours. The purification mixture may be heated for about 1.7 hours. The purification mixture may be heated for about 1.8 hours. The purification mixture may be heated for about 1.9 hours. The purification mixture may be heated for about 2 hours.

The purification mixture may be cooled to about −10° C. to about 25° C. The purification mixture may be cooled to about −5° C. to about 25° C. The purification mixture may be cooled to about −5° C. to about 20° C. The purification mixture may be cooled to about −5° C. to about 10° C. The purification mixture may be cooled to about −5° C. to about 5° C. The purification mixture may be cooled to about 0° C. to about 25° C. The purification mixture may be cooled to about 0° C. to about 20° C. The purification mixture may be cooled to about 0° C. to about 15° C. The purification mixture may be cooled to about 0° C. to about 10° C. The purification mixture may be cooled to about 0° C. to about 5° C. The purification mixture may be cooled to about 0° C. The purification mixture may be cooled to about −5° C. The purification mixture may be cooled to about −1° C. The purification mixture may be cooled to about 0° C. The purification mixture may be cooled to about 1° C. The purification mixture may be cooled to about 2° C. The purification mixture may be cooled to about 3° C. The purification mixture may be cooled to about 4° C. The purification mixture may be cooled to about 5° C. The purification mixture may be cooled to about 10° C. The purification mixture may be cooled to about 15° C. The purification mixture may be cooled to about 20° C. The purification mixture may be cooled to about 25° C.

The purification mixture may be cooled for at least 20 min. The purification mixture may be cooled for at least 30 min. The purification mixture may be cooled for at least 40 min. The purification mixture may be cooled for at least 50 min. The purification mixture may be cooled for at least 60 min. The purification mixture may be cooled for at least 80 min. The purification mixture may be cooled for at least 100 min. The purification mixture may be cooled for at least 120 min. The purification mixture may be cooled for at least 140 min. The purification mixture may be cooled for at least 160 min. The purification mixture may be cooled for about 20 min. The purification mixture may be cooled for about 30 min. The purification mixture may be cooled for about 40 min. The purification mixture may be cooled for about 50 min. The purification mixture may be cooled for about 1 hour. The purification mixture may be cooled for about 1.25 hours. The purification mixture may be cooled for about 1.5 hours. The purification mixture may be cooled for about 1.75 hours. The purification mixture may be cooled for about 2 hours. The purification mixture may be cooled for about 2.25 hours. The purification mixture may be cooled for about 2.5 hours. The purification mixture may be cooled for about 2.75 hours. The purification mixture may be cooled for about 3 hours.

The filtering may include washing the filter cake including the compound with a washing solvent. The washing solvent may be 2-butanone or tert-butyl methyl ether. The washing solvent may be 2-butanone. The washing solvent may be tert-butyl methyl ether. The compound may be dried following exposure to the washing solvent. The drying may be performed under vacuum conditions.

In another aspect is a method for purifying a compound having the formula:

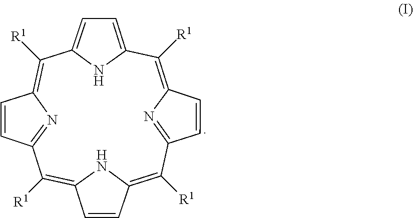

The method includes dissolving a compound of formula (I) in a purifying solvent in a reaction vessel to form a purifying mixture. The purifying mixture is heated. The purifying mixture is cooled. The purifying mixture is dried thereby purifying a compound of formula (I). The purifying mixture may be cooled after it is heated. The purifying solvent may be dimethylformamide. The purifying mixture may also include a second solvent. The second solvent may be an organic solvent. The second solvent may be dichloromethane. The compound of formula (I) may be dissolved in the second solvent to form a mixture and the purifying solvent added to the mixture before heating.

The purifying mixture may be heated to about 100° C. to about 200° C. The purifying mixture may be heated to about 110° C. to about 190° C. The purifying mixture may be heated to about 120° C. to about 180° C. The purifying mixture may be heated to about 130° C. to about 170° C. The purifying mixture may be heated to about 140° C. to about 160° C. The purifying mixture may be heated to about 125° C. to about 200° C. The purifying mixture may be heated to about 125° C. to about 175° C. The purifying mixture may be heated to about 125° C. to about 150° C. The purifying mixture may be heated to about 140° C. to about 175° C. The purifying mixture may be heated to about 140° C. to about 160° C. The purifying mixture may be heated to about 100° C. The purifying mixture may be heated to about 110° C. The purifying mixture may be heated to about 120° C. The purifying mixture may be heated to about 130° C. The purifying mixture may be heated to about 140° C. The purifying mixture may be heated to about 150° C. The purifying mixture may be heated to about 160° C. The purifying mixture may be heated to about 170° C. The purifying mixture may be heated to about 180° C. The purifying mixture may be heated to about 190° C. The purifying mixture may be heated to about 200° C.

The purifying mixture may be heated for at least 20 min. The purifying mixture may be heated for at least 20 min. The purifying mixture may be heated for at least 30 min. The purifying mixture may be heated for at least 40 min. The purifying mixture may be heated for at least 50 min. The purifying mixture may be heated for at least 60 min. The purifying mixture may be heated for at least 70 min. The purifying mixture may be heated for at least 80 min. The purifying mixture may be heated for at least 90 min. The purifying mixture may be heated for at least 100 min. The purifying mixture may be heated for at least 110 min. The purifying mixture may be heated for at least 120 min. The purifying mixture may be heated for about 20 min. The purifying mixture may be heated for about 30 min. The purifying mixture may be heated for about 40 min. The purifying mixture may be heated for about 50 min. The purifying mixture may be heated for about 1 hour. The purifying mixture may be heated for about 1.1 hours. The purifying mixture may be heated for about 1.2 hours. The purifying mixture may be heated for about 1.3 hours. The purifying mixture may be heated for about 1.4 hours. The purifying mixture may be heated for about 1.5 hours. The purifying mixture may be heated for about 1.6 hours. The purifying mixture may be heated for about 1.7 hours. The purifying mixture may be heated for about 1.8 hours. The purifying mixture may be heated for about 1.9 hours. The purifying mixture may be heated for about 2 hours.

The purifying mixture may be cooled to about 0° C. to about 50° C. The purifying mixture may be cooled to about 10° C. to about 40° C. The purifying mixture may be cooled to about 20° C. to about 30° C. The purifying mixture may be cooled to about 15° C. to about 30° C. The purifying mixture may be cooled to about 10° C. to about 30° C. The purifying mixture may be cooled to about 5° C. to about 30° C. The purifying mixture may be cooled to about 20° C. to about 50° C. The purifying mixture may be cooled to about 20° C. to about 40° C. The purifying mixture may be cooled to about 20° C. to about 30° C. The purifying mixture may be cooled to about 20° C. to about 25° C. The purifying mixture may be cooled to about 0° C. The purifying mixture may be cooled to about 5° C. The purifying mixture may be cooled to about 10° C. The purifying mixture may be cooled to about 15° C. The purifying mixture may be cooled to about 20° C. The purifying mixture may be cooled to about 25° C. The purifying mixture may be cooled to about 30° C. The purifying mixture may be cooled to about 40° C. The purifying mixture may be cooled to about 50° C.

The purifying mixture may be filtered following cooling. The filtering may include washing the filter cake including the compound with dimethylformamide.

### III. FORMULATIONS

Also provided herein is a pharmaceutical formulation that includes water and a compound having the formula

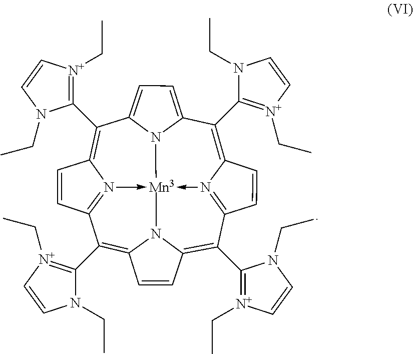

The pharmaceutical formulation may include less than about 10% to less than about 1% Mn(II). The pharmaceutical formation may include less than about 8% to less than about 1% Mn(II). The pharmaceutical formation may include less than about 5% to less than about 1% Mn(II). The pharmaceutical formulation may include less than about 10, 9, 8, 7, 6, 5, 4, 3, 2, 1% Mn(II). The pharmaceutical formulation may include less than about 10% Mn(II). The pharmaceutical formulation may include less than about 5% Mn(II). The pharmaceutical formulation may include less than about 1% Mn(II).

Mn³ is as described herein and represents the oxidation state of the Mn (e.g. Mn(III)).

The pharmaceutical formulation may have a pH of about 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 or 7.0. The pharmaceutical formulation may have a pH of about 3.5 to about 7.0. The pharmaceutical formulation may have a pH of about 3.5, 4.0, 4.5, 5.0, 5.5, 6.0, 6.5, or 7.0. The pharmaceutical formulation may have a pH of about 3.5 to about 5.5. The pharmaceutical formulation may have a pH of about 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5. The pharmaceutical formulation may consist essentially of water and a compound described herein, including embodiments thereof. The compound may be a compound of formula (VI) including embodiments thereof. The pharmaceutical formulation may include water, the compound, and pH adjustment ions. The pH adjustment ions may result from dissolution of an acid or base, such as HCl, NaOH or ascorbic acid. When the pharmaceutical formulation includes a buffer, the buffer may be, for example, citrate, phosphate, acetate, or ammonium buffers. In embodiments, the pharmaceutical formulation does not include a buffer (i.e. the compound is not a buffer itself). The pharmaceutical formulation may not include a pharmaceutical excipient.

The pharmaceutical formulation may be at a concentration of about 25 mg/mL to about 600 mg/mL. The concentration may be about 65 mg/mL. The concentration may be about 75 mg/mL. The concentration may be about 100 mg/mL. The concentration may be about 150 mg/mL. The concentration may be about 200 mg/mL. The concentration may be about 250 mg/mL. The concentration may be about 300 mg/mL. The concentration may be about 350 mg/mL. The concentration may be about 400 mg/mL. The pharmaceutical formulation concentration may be stored at 5° C. or 25° C.

### IV. KITS

In another aspect is a container including a plurality of compounds having the formula:

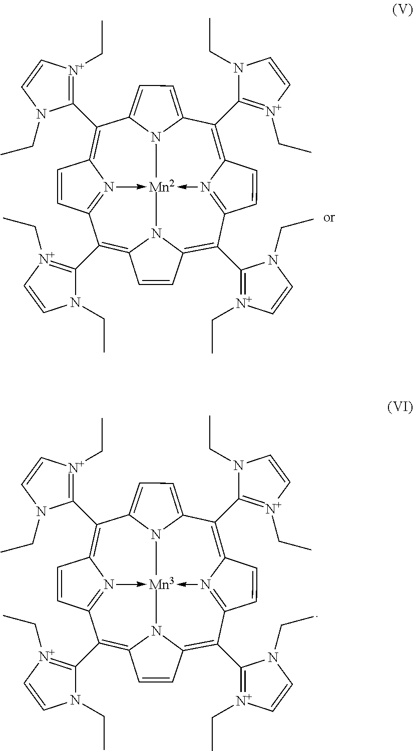

At least 60% of the plurality of compounds have formula (VI). As set forth herein, Mn² represents the oxidation state of the compound (i.e. Mn² is the Mn(II) oxidation state). Likewise, Mn³ represents the oxidation state of the compound (i.e. Mn³ is the Mn(III) oxidation state).

At least 60, 65, 70, 75, 80, 85, 90, 95, or 100% of the plurality of compounds may have formula (VI). At least 60% of the plurality of compounds may have formula (VI). At least 65% of the plurality of compounds may have formula (VI). At least 70% of the plurality of compounds may have formula (VI). At least 75% of the plurality of compounds may have formula (VI). At least 80% of the plurality of compounds may have formula (VI). At least 85% of the plurality of compounds may have formula (VI). At least 90% of the plurality of compounds may have formula (VI). At least 91% of the plurality of compounds may have formula (VI). At least 92% of the plurality of compounds may have formula (VI). At least 93% of the plurality of compounds may have formula (VI). At least 94% of the plurality of compounds may have formula (VI). At least 95% of the plurality of compounds may have formula (VI). At least 96% of the plurality of compounds may have formula (VI). At least 97% of the plurality of compounds may have formula (VI). At least 98% of the plurality of compounds may have formula (VI). At least 99% of the plurality of compounds may have formula (VI).

The compound having formula (V) may be oxidized to the compound having formula (VI) by exposure to water after less than 1 hour. The compound having formula (V) may be oxidized to the compound having formula (VI) by exposure to water after about 1, 5, 10, 15, 20, 24, 30, 35, 40, 45, 48, 50, 55, 60, 65, 70, 75, 80, 85, 90, or about 96 hours. The compound having formula (V) may be oxidized to the compound having formula (VI) by exposure to water after about 1 hour to about 96 hours. The oxidation of the compound of formula (V) to the compound of formula (VI) may occur after exposure to water after about 16 to about 96 hours. The oxidation of the compound of formula (V) to the compound of formula (VI) may occur after exposure to water. The oxidation of the compound may occur after exposure to water for about 1, 2, 3, 4, 5, 6, 7, 8, 9, 10, 11, 12, 13, 14, 15, 16, 17, 18, 19, 20 ,21, 22, 23, 24, 25, 26, 27, 28, 29, 30, 31, 32, 33, 34, 35, 36, 37, 38, 39, 40, 41, 42, 43, 44, 45, 46, 47, or 48 hours. The oxidation may occur after about 1 h exposure time. The oxidation may occur after about 2-4 h exposure time. The oxidation may occur after about 4-8 h exposure time. The oxidation may occur after about a 8-16 h exposure time. The oxidation may occur after about a 16-24 h exposure time. The oxidation may occur after about a 16-48 h exposure time. The oxidation may occur after about a 24-48 h exposure time.

The oxidation may occur after about exposing the compound to water for about 30 min. The oxidation may occur after about exposing the compound to water for about 1 hour. The oxidation may occur after about exposing the compound to water for about 2 hours. The oxidation may occur after about exposing the compound to water for about 3 hours. The oxidation may occur after about exposing the compound to water for about 4 hours. The oxidation may occur after about exposing the compound to water for about 5 hours. The oxidation may occur after about exposing the compound to water for about 6 hours. The oxidation may occur after about exposing the compound to water for about 7 hours. The oxidation may occur after about exposing the compound to water for about 8 hours. The oxidation may occur after about exposing the compound to water for about 9 hours. The oxidation may occur after about exposing the compound to water for about 10 hours. The oxidation may occur after about exposing the compound to water for about 11 hours. The oxidation may occur after about exposing the compound to water for about 12 hours. The oxidation may occur after about exposing the compound to water for about 13 hours. The oxidation may occur after about exposing the compound to water for about 14 hours. The oxidation may occur after about exposing the compound to water for about 15 hours. The oxidation may occur after about exposing the compound to water for about 16 hours. The oxidation may occur after about exposing the compound to water for about 20 hours. The oxidation may occur after about exposing the compound to water for about 24 hours. The oxidation may occur after about exposing the compound to water for about 30 hours. The oxidation may occur after about exposing the compound to water for about 35 hours. The oxidation may occur after about exposing the compound to water for about 40 hours. The oxidation may occur after about exposing the compound to water for about 48 hours.

The oxidation of a compound having formula (V) to a compound having formula (VI) may occur at atmospheric oxygen concentrations. The oxidation of a compound having formula (V) to a compound having formula (VI) may occur at an oxygen concentration lower than atmospheric concentrations as described herein, including embodiments thereof. The oxidation of a compound having formula (V) to a compound having formula (VI) may occur at oxygen concentrations greater than atmospheric concentrations. The rate of oxidation of a compound having formula (V) to a compound having formula (VI) may be accelerated at higher oxygen concentrations. Oxygen concentrations greater than atmospheric concentrations may accelerate the rate of oxidation to the Mn(III) oxidation state.

The plurality of compounds may include a counterion selected from the group consisting of a halogen anion, SCN⁻, SO₄⁻², HSO₄⁻, H₂PO₄⁻, H₂PO₄⁻², PO₄⁻³, NO₃⁻, PF₆⁻, or BF₄⁻. The halogen anion may be F⁻, Cl⁻, Br⁻, or I⁻. The counterion may be Cl⁻. One skilled in art would recognize that any appropriate counterion could be present. The counterion may be exchanged during a precipitation step with an ammonium salt, as described herein. The ammonium salt may be Bu₄NCl or NH₄PF₆.

The container may include the plurality of compounds in water thereby forming a pharmaceutical formulation. When in water, the pharmaceutical formulation within the container is at a pH as described herein, including embodiments thereof. For example, the formulation within the container may be at a pH of from about 3.5 to about 7.0. The pharmaceutical formulation within the container may be at a pH of about 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 or 7.0. The pharmaceutical formulation within the container may be at a pH of about 3.5, 4.0, 4.5, 5.0, 5.5, 6.0, 6.5, or 7.0. The pharmaceutical formulation within the container may be at a pH of about 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5. The pharmaceutical formulation is at a pH of from about 3.5 to about 5.5.

The pharmaceutical formulation supplied in the container may consist essentially of water and a compound as described herein, including embodiments thereof. The compound may be a compound of formula (VI). The container of claim including the pharmaceutical formulation may include compose of water, a compound as described herein, including embodiments thereof, and pH adjustment ions. The compound may be a compound of formula (VI). The pH adjustment ions may result from dissolution of an acid or base, such as HCl, NaOH, or ascorbic acid. When the pharmaceutical formulation supplied in the container includes a buffer, the buffer may be known by those skilled in the art, including, for example, citrate, phosphate, acetate, or ammonium buffers. The pharmaceutical formulation supplied in the container may not include a buffer (i.e. the compound is not a buffer itself). The pharmaceutical formulation supplied in the container may not include a pharmaceutical excipient.

The pharmaceutical formulation may be at a concentration of about 25 mg/mL to about 600 mg/mL. The concentration may be about 65 mg/mL. The concentration may be about 75 mg/mL. The concentration may be about 100 mg/mL. The concentration may be about 150 mg/mL. The concentration may be about 200 mg/mL. The concentration may be about 250 mg/mL. The concentration may be about 300 mg/mL. The concentration may be about 350 mg/mL. The concentration may be about 400 mg/mL. The pharmaceutical formulation concentration may be stored at 5° C. or 25° C.

### V. CRYSTAL COMPOSITIONS AND METHODS

In another aspect is a crystal that includes a compound having the formula:

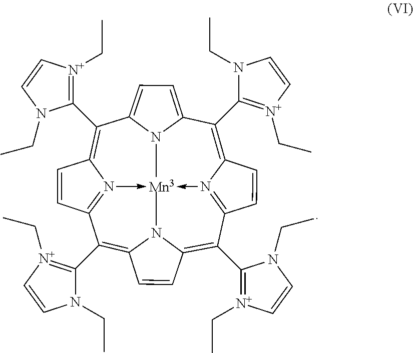

Mn³ is as described herein and represents the oxidation state of the Mn (e.g. Mn(III)). The crystal may be a hydrate, formed using methods as described herein. The crystal having formula (VI) may have about 14% water content at about 20% relative humidity (RH). The crystal having formula (VI) may have about 15% water content at about 40% RH. The crystal having formula (VI) may have about 17% water content at about 75% RH. The crystal having formula (VI) may have about 0% water content at about less than 2% RH. The crystal may be a hydrate.

In another aspect is a crystalline form of [5,10,15,20-tetrakis(1,3-diethylimidazolium-2-yl)porphyrinato]manganese(III) chloride hydrate complex. The crystalline form is characterized by an x-ray powder diffraction spectrum (XRPD). The x-ray powder diffraction spectrum includes angle 2θ peaks at about 6.9±0.2, 8.2±0.2, 9.5±0.2, 11.4±0.2, 12.8±0.2, 14.5±0.2, 15.0±0.2, 16.1±0.2, 16.3±0.2, 18.1±0.2, 20.3±0.2, 23.5±0.2, 24.8±0.2, 25.6±0.2, 26.5±0.2, and 29.2±0.2. Values for angle 2θ peaks provided herein are those values resulting from the use of a Cu Kα radiation source (1.54 Å). The crystalline form may further include the x-ray powder diffraction spectrum having angle 2θ peaks at about 13.8±0.2, 17.4±0.2, 19.0±0.2, 19.4±0.2, 20.7±0.2, 21.1±0.2, 21.5±0.2, 22.0±0.2, 22.5±0.2, 22.8±0.2, 26.9±0.2, 27.6±0.2, 28.5±0.2, 30.2±0.2, 30.5±0.2, 31.2±0.2, 37.3±0.2, 38.5±0.2, and 41.1±0.2.

The crystalline form may include the x-ray powder diffraction spectrum having angle 2θ peaks at about 6.9±0.2, 8.2±0.2, 9.5±0.2, 11.4±0.2, 12.8±0.2, 13.8±0.2, 14.5±0.2, 15.0±0.2, 16.1±0.2, 16.3±0.2, 17.4±0.2, 18.1±0.2, 19.0±0.2, 19.4±0.2, 20.3±0.2, 20.7±0.2, 21.1±0.2, 21.5±0.2, 22.0±0.2, 22.5±0.2, 22.8±0.2, 23.5±0.2, 24.8±0.2, 25.6±0.2, 26.5±0.2, 26.9±0.2, 27.6±0.2, 28.5±0.2, 29.2±0.2, 30.2±0.2, 30.5±0.2, 31.2±0.2, 37.3±0.2, 38.5±0.2, and 41.1±0.2.

In another aspect is a crystalline form of [5,10,15,20-tetrakis(1,3-diethylimidazolium-2-yl)porphyrinato]manganese(III) chloride hydrate complex. The crystalline form is characterized by an x-ray powder diffraction spectrum. The x-ray powder diffraction spectrum includes d spacings at about 12.85, 10.82, 9.28, 7.78, 6.91, 6.11, 5.91, 5.49, 5.42, 4.89, 4.37, 3.78, 3.58, 3.47, 3.36, and 3.06. The d spacing values should be understood to include variances associated with X-ray diffraction spectroscopy. The x-ray powder diffraction spectrum is obtained using a Cu Kα radiation source (1.54 Å). The crystalline form may further include the x-ray powder diffraction spectrum having d spacings at about, 7.57, 6.44, 5.10, 4.67, 4.58, 4.29, 4.2, 4.13, 4.05, 3.96, 3.89, 3.31, 3.22, 3.13, 2.96, 2.93, 2.86, 2.41, 2.34, and 2.19.

The crystalline form may include the x-ray powder diffraction spectrum having d spacings at about 12.85, 10.82, 9.28, 7.78, 7.57, 6.91, 6.44, 6.11, 5.91, 5.49, 5.42, 5.1, 4.89, 4.67, 4.58, 4.37, 4.29, 4.2, 4.13, 4.05, 3.96, 3.89, 3.78, 3.58, 3.47, 3.36, 3.31, 3.22, 3.13, 3.06, 2.96, 2.93, 2.86, 2.41, 2.34, and 2.19.

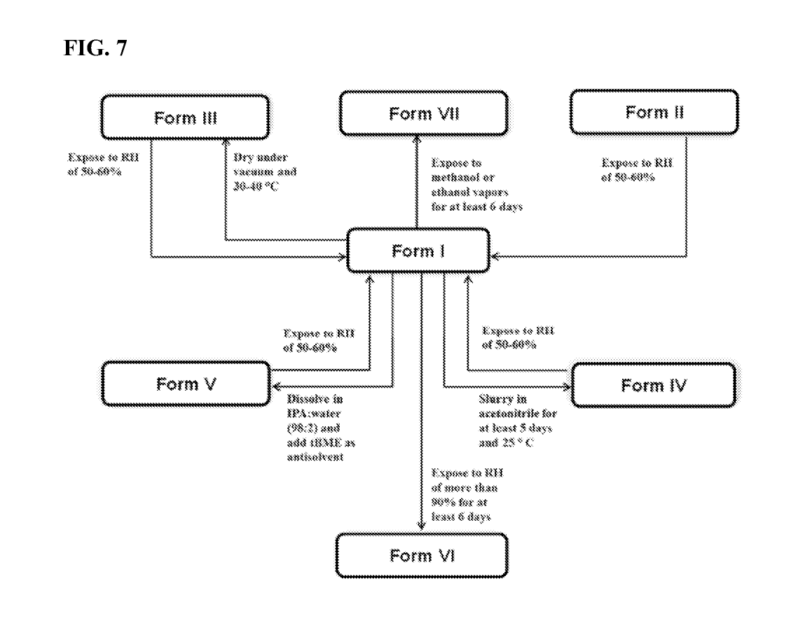

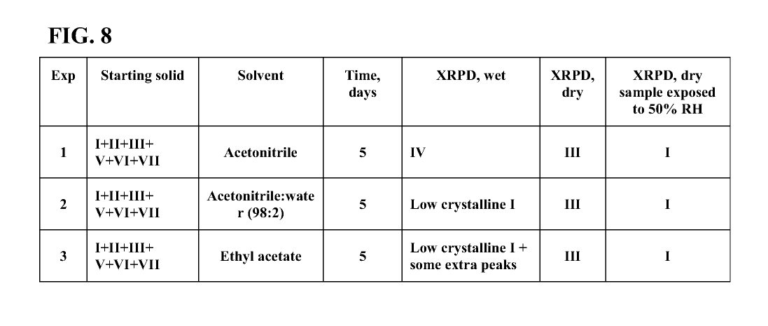

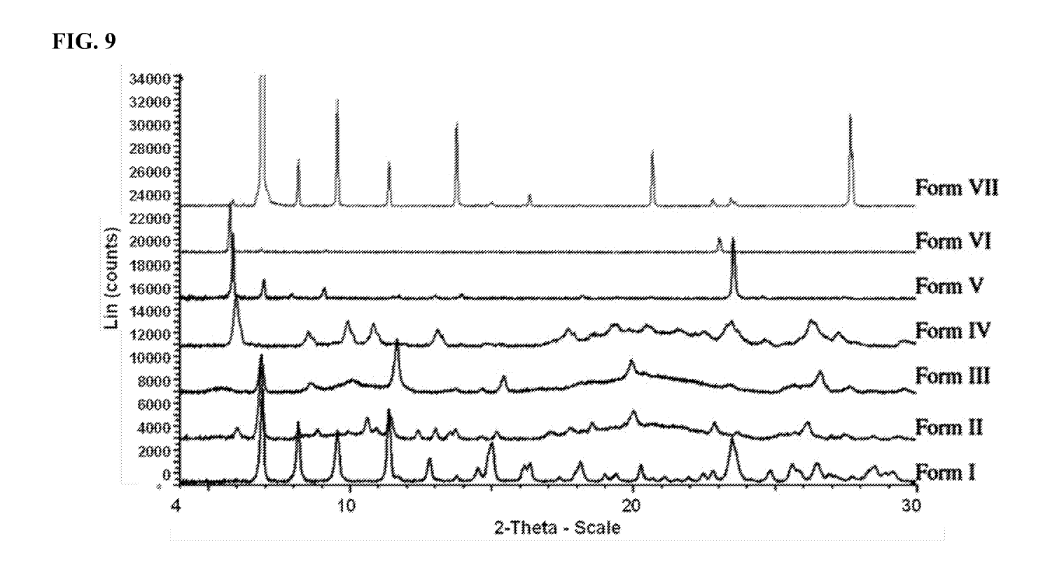

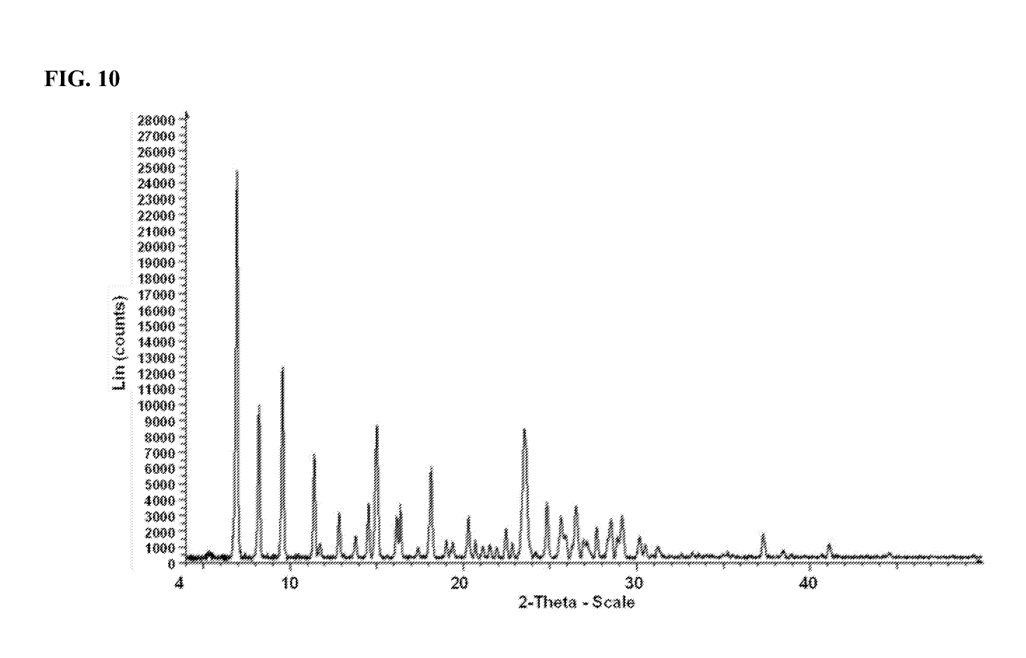

The recrystallization may yield multiple polymorphs of formula (VI). The polymorphic forms of the compound of formula (VI), including embodiments thereof, may result for example, from the isolation technique used, conditions of exposure to organic solvents, percentages of relative humidity, and/or time periods for such exposure, as set forth in Table 1.2. The polymorphic states may be form I, form II, form III, form IV, form V, form VI, or form VII. Forms II, III, IV, V, VI, and VII may be converted to form I. The interconversion of the different polymorphic forms of formula (VI) may proceed under the conditions set forth in Table 1.2 or in FIG. 7. Form I may be the most stabile form of a compound having formula (IV).

The crystal form may be form I. Form I may have the x-ray powder diffraction spectrum having angle 2θ peaks of about 6.9±0.2, 8.2±0.2, 9.5±0.2, 11.4±0.2, 12.8±0.2, 14.5±0.2, 15.0±0.2, 16.1±0.2, 16.3±0.2, 18.1±0.2, 20.3±0.2, 23.5±0.2, 24.8±0.2, 25.6±0.2, 26.5±0.2, and 29.2±0.2. Values for angle 2θ peaks provided herein are those values resulting from the use of a Cu Kα radiation source (1.54 Å). Form I may further include the x-ray powder diffraction spectrum having angle 2θ peaks at about 13.8±0.2, 17.4±0.2, 19.0±0.2, 19.4±0.2, 20.7±0.2, 21.1±0.2, 21.5±0.2, 22.0±0.2, 22.5±0.2, 22.8±0.2, 26.9±0.2, 27.6±0.2, 28.5±0.2, 30.2±0.2, 30.5±0.2, 31.2±0.2, 37.3±0.2, 38.5±0.2, and 41.1±0.2.

Form I may include the x-ray powder diffraction spectrum having angle 2θ peaks at about 6.9±0.2, 8.2±0.2, 9.5±0.2, 11.4±0.2, 12.8±0.2, 13.8±0.2, 14.5±0.2, 15.0±0.2, 16.1±0.2, 16.3±0.2, 17.4±0.2, 18.1±0.2, 19.0±0.2, 19.4±0.2, 20.3±0.2, 20.7±0.2, 21.1±0.2, 21.5±0.2, 22.0±0.2, 22.5±0.2, 22.8±0.2, 23.5±0.2, 24.8±0.2, 25.6±0.2, 26.5±0.2, 26.9±0.2, 27.6±0.2, 28.5±0.2, 29.2±0.2, 30.2±0.2, 30.5±0.2, 31.2±0.2, 37.3±0.2, 38.5±0.2, and 41.1±0.2.

Form I may include the x-ray powder diffraction spectrum including d spacings at about 12.85, 10.82, 9.28, 7.78, 6.91, 6.11, 5.91, 5.49, 5.42, 4.89, 4.37, 3.78, 3.58, 3.47, 3.36, and 3.06. The d spacing values should be understood to include variances associated with X-ray diffraction spectroscopy. The x-ray powder diffraction spectrum is obtained using a Cu Kα radiation source (1.54 Å). Form I may further include the x-ray powder diffraction spectrum having d spacings at about, 7.57, 6.44, 5.10, 4.67, 4.58, 4.29, 4.2, 4.13, 4.05, 3.96, 3.89, 3.31, 3.22, 3.13, 2.96, 2.93, 2.86, 2.41, 2.34, and 2.19.

Form I may include the x-ray powder diffraction spectrum having d spacings at about 12.85, 10.82, 9.28, 7.78, 7.57, 6.91, 6.44, 6.11, 5.91, 5.49, 5.42, 5.10, 4.89, 4.67, 4.58, 4.37, 4.29, 4.2, 4.13, 4.05, 3.96, 3.89, 3.78, 3.58, 3.47, 3.36, 3.31, 3.22, 3.13, 3.06, 2.96, 2.93, 2.86, 2.41, 2.34, and 2.19.

In another aspect is a crystalline form of [5,10,15,20-tetrakis(1,3-diethylimidazolium-2-yl)porphyrinato]manganese(III) chloride hydrate complex, wherein the crystal form is Form II. Form II may have the x-ray powder diffraction spectrum having angle 2θ peaks of about 26.2±0.2, 22.9±0.2, 20.0±0.2, 18.6±0.2, 15.2±0.2, 13.7±0.2, 13.5±0.2, 13.0±0.2, 12.4±0.2, 11.4±0.2, 10.6±0.2, 8.9±0.2, 6.8±0.2, and 6.0±0.2. Values for angle 2θ peaks provided herein are those values resulting from the use of a Cu Kα radiation source (1.54 Å). Form II may further include the x-ray powder diffraction spectrum having angle 2θ peaks of about 29.4±0.2, 28.5±0.2, 27.5±0.2, 27.0±0.2, 25.7±0.2, 25.2±0.2, 23.7±0.2, 17.8±0.2, 17.1±0.2, 14.6±0.2, 10.9±0.2, 9.9±0.2, and 8.2±0.2.

Form II may have the x-ray powder diffraction spectrum having angle 2θ peaks of about 29.4±0.2, 28.5±0.2, 27.5±0.2, 27±0.2, 26.2±0.2, 25.7±0.2, 25.2±0.2, 23.7±0.2, 22.9±0.2, 20.0±0.2, 18.6±0.2, 17.8±0.2, 17.1±0.2, 15.2±0.2, 14.6±0.2, 13.73±0.2, 13.5±0.2, 13.0±0.2,12.4±0.2, 11.±0.2, 10.9±0.2, 10.6±0.2, 9.9±0.2, 8.9±0.2, 8.2±0.2, 6.8±0.2, and 6.0±0.2.

Form II may include the x-ray powder diffraction spectrum including d spacings at about 14.74, 12.93, 9.99, 8.34, 7.74, 7.14, 6.80, 6.55, 6.45, 5.83, 4.78, 4.43, 3.89, and 3.40. The d spacing values should be understood to include variances associated with X-ray diffraction spectroscopy. Form II may further include the x-ray powder diffraction spectrum including d spacings at about 10.82, 8.90, 8.10, 6.05, 5.19, 4.98, 3.75, 3.54, 3.47, 3.30, 3.24, 3.13, and 3.04.

Form II may include the x-ray powder diffraction spectrum including d spacings at about 14.74, 12.93, 10.82, 9.99, 8.9, 8.34, 8.1, 7.74, 7.14, 6.8, 6.55, 6.45, 6.05, 5.83, 5.19, 4.98, 4.78, 4.43, 3.89, 3.75, 3.54, 3.47, 3.40, 3.30, 3.24, 3.13, and 3.04.

In another aspect is a crystalline form of [5,10,15,20-tetrakis(1,3-diethylimidazolium-2-yl)porphyrinato]manganese(III) chloride hydrate complex, wherein the crystal form is Form III. Form III may have the x-ray powder diffraction spectrum having angle 2θ peaks of about 27.7±0.2, 26.6±0.2, 19.9±0.2, 15.4±0.2, 14.7±0.2, 11.6±0.2, 10.1±0.2, 8.6±0.2, and 6.9±0.2. Values for angle 2θ peaks provided herein are those values resulting from the use of a Cu Kα radiation source (1.54 Å). Form III may further include the x-ray powder diffraction spectrum having angle 2θ peaks of about 29.6±0.2, 25.7±0.2, 23.4±0.2, 20.4±0.2, and 13.7±0.2.

Form III may have the x-ray powder diffraction spectrum having angle 2θ peaks of about 29.6±0.2, 27.7±0.2, 26.6±0.2, 25.7±0.2, 23.4±0.2, 20.4±0.2, 19.9±0.2, 15.4±0.2, 14.7±0.2, 13.7±0.2, 11.6±0.2, 10.1±0.2, 8.6±0.2, and 6.9±0.2.

Form III may include the x-ray powder diffraction spectrum including d spacings at about 12.89, 10.27, 8.79, 7.60, 6.04, 5.74, 4.45, 3.35, and 3.22. The d spacing values should be understood to include variances associated with X-ray diffraction spectroscopy. Form III may further include the x-ray powder diffraction spectrum including d spacings at about 6.45, 4.35, 3.80, 3.46, and 3.02.

Form III may include the x-ray powder diffraction spectrum including d spacings at about 12.89, 10.27, 8.79, 7.60, 6.45, 6.04, 5.74, 4.45, 4.35, 3.80, 3.46, 3.35, 3.22 and 3.02.

In another aspect is a crystalline form of [5,10,15,20-tetrakis(1,3-diethylimidazolium-2-yl)porphyrinato]manganese(III) chloride hydrate complex, wherein the crystal form is Form IV. Form IV may have the x-ray powder diffraction spectrum having angle 2θ peaks of about 29.5±0.2, 27.3±0.2, 26.3±0.2, 24.7±0.2, 23.5±0.2, 22.5±0.2, 21.6±0.2, 20.5±0.2, 19.3±0.2, 17.7±0.2, 13.1±0.2, 10.8±0.2, 9.9±0.2, 8.5±0.2, and 6.0±0.2. Values for angle 2θ peaks provided herein are those values resulting from the use of a Cu Kα radiation source (1.54 Å). Form IV may further include the x-ray powder diffraction spectrum having angle 2θ peaks of about 32.6±0.2, 19.8±0.2, 18.6±0.2, and 14.8±0.2.

Form IV may have the x-ray powder diffraction spectrum having angle 2θ peaks of about 32.6±0.2, 29.5±0.2, 27.3±0.2, 26.3±0.2, 24.7±0.2, 23.5±0.2, 22.5±0.2, 21.6±0.2, 20.5±0.2, 19.8±0.2, 19.3±0.2, 18.6±0.2, 17.7±0.2, 14.8±0.2, 13.1±0.2, 10.8±0.2, 9.9±0.2, 8.5±0.2, and 6.0±0.2.

In another aspect is a crystalline form of [5,10,15,20-tetrakis(1,3-diethylimidazolium-2-yl)porphyrinato]manganese(III) chloride hydrate complex, wherein the crystal form is Form V. Form V may have the x-ray powder diffraction spectrum having angle 2θ peaks of about 23.5±0.2, 9.1±0.2, 6.9±0.2, and 5.8±0.2. Values for angle 2θ peaks provided herein are those values resulting from the use of a Cu Kα radiation source (1.54 Å). Form V may further include the x-ray powder diffraction spectrum having angle 2θ peaks of about 27.5±0.2, 24.6±0.2, 18.2±0.2, 13.9±0.2, 13.0±0.2, 11.7±0.2, and 7.9±0.2.

Form V may have the x-ray powder diffraction spectrum having angle 2θ peaks of about 27.5±0.2, 24.6±0.2, 23.5±0.2, 18.2±0.2, 13.9±0.2, 13.0±0.2, 11.7±0.2, 9.1±0.2, 7.9±0.2, 6.9±0.2, and 5.8±0.2.

Form V may include the x-ray powder diffraction spectrum including d spacings at about 15.12, 12.74, 9.75, and 3.78. The d spacing values should be understood to include variances associated with X-ray diffraction spectroscopy. Form V may further include the x-ray powder diffraction spectrum including d spacings at about 11.14, 7.55, 6.81, 6.36, 4.87, 3.62, and 3.24.

Form V may include the x-ray powder diffraction spectrum including d spacings at about 15.12, 12.74, 11.14, 9.75, 7.55, 6.81, 6.36, 4.87, 3.78, 3.62, and 3.24.

In another aspect is a crystalline form of [5,10,15,20-tetrakis(1,3-diethylimidazolium-2-yl)porphyrinato]manganese(III) chloride hydrate complex, wherein the crystal form is Form VI. Form VI may have the x-ray powder diffraction spectrum having angle 2θ peaks of about 27.7±0.2, 23.6±0.2, 23.1±0.2, 20.7±0.2, 6.9±0.2, and 5.8±0.2. Values for angle 2θ peaks provided herein are those values resulting from the use of a Cu Kα radiation source (1.54 Å). Form VI may further include the x-ray powder diffraction spectrum having angle 2θ peaks of about 29.2±0.2, 28.9±0.2, 27.1±0.2, 26.5±0.2, 26.2±0.2, 24.8±0.2, 22.4±0.2, 22.2±0.2, 21.5±0.2, 20.3±0.2, 18.1±0.2, 17.3±0.2, 16.3±0.2, 14.9±0.2, 13.8±0.2, 11.5±0.2, and 9.2±0.2.

Form VI may have the x-ray powder diffraction spectrum having angle 2θ peaks of about 29.2±0.2, 28.9±0.2, 27.7±0.2, 27.1±0.2, 26.5±0.2, 26.2±0.2, 24.8±0.2, 23.1±0.2, 22.4±0.2, 22.2±0.2, 21.5±0.2, 20.7±0.2, 20.3±0.2, 18.1±0.2, 17.3±0.2, 16.3±0.2, 14.9±0.2, 13.8±0.2, 11.5±0.2, 9.2±0.2, 6.9±0.2, and 5.8±0.2.

In another aspect is a crystalline form of [5,10,15,20-tetrakis(1,3-diethylimidazolium-2-yl)porphyrinato]manganese(III) chloride hydrate complex, wherein the crystal form is Form VII.

Form VII may have the x-ray powder diffraction spectrum having angle 2θ peaks of about 27.7±0.2, 20.7±0.2, 13.8±0.2, 11.4±0.2, 9.5±0.2, 8.2±0.2, and 6.9±0.2. Values for angle 2θ peaks provided herein are those values resulting from the use of a Cu Kα radiation source (1.54 Å). Form VII may further include the x-ray powder diffraction spectrum having angle 2θ peaks of about 23.5±0.2, 22.8±0.2, 16.3±0.2, and 5.9±0.2.

Form VII may have the x-ray powder diffraction spectrum having angle 2θ peaks of about 27.7±0.2, 23.5±0.2, 22.8±0.2, 20.7±0.2, 16.3±0.2, 13.8±0.2, 11.4±0.2, 9.5±0.2, 8.2±0.2, 6.9±0.2, and 5.9±0.2.

Form VII may include the x-ray powder diffraction spectrum including d spacings at about 12.84, 10.83, 9.26, 7.77, 6.43, 4.29, and 3.22. The d spacing values should be understood to include variances associated with X-ray diffraction spectroscopy. Form VII may further include the x-ray powder diffraction spectrum including d spacings at about 15.07, 5.42, 3.89, and 3.79.

Form VII may include the x-ray powder diffraction spectrum including d spacings at about 15.07, 12.84, 10.83, 9.26, 7.77, 6.43, 5.42, 4.29, 3.89, 3.79, and 3.22

TABLE 1.2
Conditions for polymorphs of compounds described herein.

| Numerical Designation | Conditions to obtain the solid form |
|---|---|
| I | Expose any of the solid forms to relative humidity of 50-60% for more than |
| | one hour |
| II | Wet cake out of reaction mixture unexposed to moisture. This is from the |
| | latest process with 3 eq. Mn (III) acetate |
| III | Drying of any of the solid forms results in this unstable solid form. Due to |
| | instability, some peaks might be shifted if the same experiment is repeated |
| | multiple times. |
| IV | Wet cake from slurrying all the solid forms in acetonitrile for at least 5 days |
| | and at room temperature. |
| V | Dissolve Form I IPA:water (98:2) and add tBME as antisolvent. Wet cake. |
| VI | Expose Form I to moisture of more than 95% for at least 6 days. A liquid. |
| VII | Expose Form I to ethanol or methanol vapors for at least 6 days. A liquid. |

Recrystallization may be performed using techniques known in the art, including, for example, evaporative crystallization, antisolvent crystallization, reactive crystallization, or vapor diffusion into solid crystallization. Crystallization of a compound of formula (VI) may be performed using evaporative crystallization. The crystallization may be performed with excess Mn present. The crystallization may be performed in one or more of solvents such as, for example, 2-propanol, acetonitrile, or water. The crystallization may be performed using a mixture of isopropanol:water (98:2) or acetonitrile:water (98:2). The solvents may yield only Form I of formula (VI). Crystallization of a compound of formula (VI) may be performed using antisolvent crystallization. The crystallization may be performed using isopropanol, ethanol, methanol, isopropanol:water (98:2), or acetonitrile:water (98:2) as a solvent. The crystallization may be performed using heptane, tert-butyl methyl ether, or ethyl acetate as an antisolvent. Antisolvent crystallization may occur via addition of the solvent followed by the antisolvent. Alternatively, antisolvent crystallization may occur via addition of the antisolvent followed by the solvent. Antisolvent crystallization may yield only Form I of formula (IV). Antisolvent crystallization may yield Form V or form VII of formula (VI). Crystallization of a compound of formula (VI) may be performed using reactive crystallization wherein the manganese salt is added as the reactive step. Precipitation may be performed using a solvent such as, for example, tert-butyl ammonium chloride. The precipitating solvent may be added instantaneously or over a period of time (e.g. about 30 minutes.). Crystallization of a compound of formula (VI) may be performed using vapor diffusion into a solid. The crystallization may be performed in one or more solvents such as, for example, acetone, tert-butyl methyl ether, ethanol, ethyl acetate, diethyl ether (DEE), acetonitrile, tetrahydrofuran, dichloromethane, 1,4-dioxane, heptane, isopropyl acetate (IPAc), methyl ethyl ketone, isopropanol, methanol, acetonitrile:water (98:2), saturated sodium hydroxide (8% relatively humidity), saturated potassium carbonate (K₂CO₃) (43% relative humidity), saturated potassium iodide (69% relative humidity), saturated sodium chloride (75% relative humidity), saturated potassium chloride (85% relative humidity), or water. The solvent may be allowed to diffuse for at least 6 days. Vapor diffusion into solid crystallization may yield Form I, Form VI, or Form VII of formula (VI).

### VI. EMBODIMENTS

Embodiment 1 A method for synthesizing a substituted porphyrin having the formula:

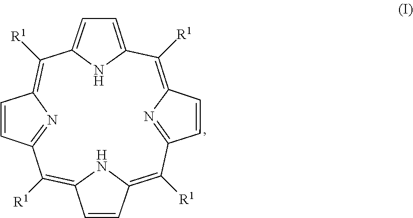

wherein R¹ is substituted or unsubstituted heterocycloalkyl or substituted or unsubstituted heteroaryl, said method comprising: (i) contacting a pyrrole with an R¹-substituted aldehyde, wherein said contacting is performed in a solvent system comprising a positive azeotrope; (ii) allowing said pyrrole to react with said R¹-substituted aldehyde in said solvent system under azeotropic distillation conditions, thereby forming a substituted-porphyrinogen; (iii) oxidizing said substituted-porphyrinogen, thereby synthesizing a substituted porphyrin having formula (I).

Embodiment 2 The method of embodiment 1 or 2, wherein said contacting is performed using about one equivalent pyrrole and about one equivalent R¹-substituted aldehyde.

Embodiment 3 The method of any one of embodiments 1 to 3, wherein R¹ is substituted or unsubstituted heteroaryl.

Embodiment 4 The method of any one of embodiments 1 to 3, wherein R¹ is substituted or unsubstituted imidazolyl, substituted or unsubstituted pyrazolyl, substituted or unsubstituted thiazolyl, or substituted or unsubstituted triazolyl.

Embodiment 5 The method of any one of embodiments 1 to 4, wherein R¹ is substituted imidazolyl.

Embodiment 6 The method of any one of embodiments 1 to 5, wherein R¹ is:

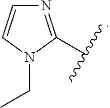 [SMILES: *c1nccn1CC]

Embodiment 7 The method of any one of embodiments 1 to 6, wherein R¹ is substituted or unsubstituted heteroaryl.

Embodiment 8 The method of any one of embodiments 1 to 7, wherein R¹ is substituted or unsubstituted pyridinyl, substituted or unsubstituted pyrazinyl, substituted or unsubstituted pyrimidinyl, or substituted or unsubstituted pyridazinyl.

Embodiment 9 The method of any one of embodiments 1 to 8, wherein said solvent system comprises a first solvent and an acid.

Embodiment 10 The method of any one of embodiments 1 to 9, wherein said first solvent is chlorobenzene, m-xylene, or toluene.

Embodiment 11 The method of any one of embodiments 1 to 10, wherein said first solvent is toluene.

Embodiment 12 The method of any one of embodiments 1 to 9, wherein said acid is a carboxylic acid.

Embodiment 13 The method of any one of embodiments 1 to 12, wherein said carboxylic acid is acetic acid, formic acid, propionic acid, valeric acid or butyric acid.

Embodiment 14 The method of any one of embodiments 1 to 13, wherein said carboxylic acid is propionic acid.

Embodiment 15 The method of any one of embodiments 1 to 14, wherein said positive azeotrope comprises water and toluene.

Embodiment 16 The method of any one of embodiments 1 to 15, wherein said substituted porphyrin has a yield of from about 6% to about 35%.

Embodiment 17 The method of any one of embodiments 1 to 16, wherein said substituted porphyrin has a yield of from about 8% to about 35%.

Embodiment 18 The method of any one of embodiments 1 to 17, wherein said substituted porphyrin has a yield of from about 10% to about 35%.

Embodiment 19 The method of any one of embodiments 1 to 18, wherein said substituted porphyrin has a yield of at least about 10%.

Embodiment 20 The method of any one of embodiments 1 to 18, wherein said substituted porphyrin has a yield of at least about 15%.

Embodiment 21 The method of any one of embodiments 1 to 18, wherein said substituted porphyrin has a yield of at least about 20%.

Embodiment 22 The method of any one of embodiments 1 to 18, wherein said substituted porphyrin has a yield of at least about 25%.

Embodiment 23 The method of any one of embodiments 1 to 18, wherein said substituted porphyrin has a yield of at least about 30%.

Embodiment 24 The method of any one of embodiments 1 to 23, wherein said reacting is performed at a temperature from about 40° C. to about 150° C.

Embodiment 25 The method of any one of embodiments 1 to 24, wherein said reacting is performed at a temperature of about 140° C.

Embodiment 26 The method of any one of embodiments 1 to 25, wherein said oxidizing is performed by exposure to air or by using an oxidant.

Embodiment 27 The method of any one of embodiments 1 to 26, wherein said oxidant is 2,3-dichloro-5,6-dicyano-1,4-benzoquinone.

Embodiment 28 The method of any one of embodiments 1 to 27, wherein said oxidizing is performed in a low oxygen environment.

Embodiment 29 The method of any one of embodiments 1 to 28, wherein said oxidizing is performed in the absence of an exogenous oxidant.

Embodiment 30 The method of any one of embodiments 1 to 29, wherein the compound of formula (I) has the formula:

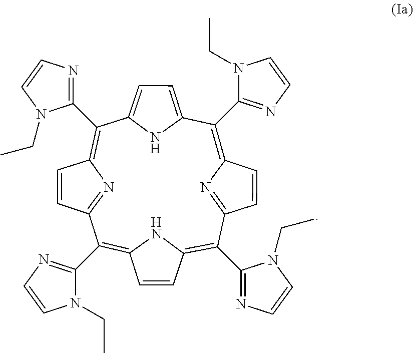 [SMILES: CCn1ccnc1-c1c2nc(c(-c3nccn3CC)c3ccc([nH]3)c(-c3nccn3CC)c3nc(c(-c4nccn4CC)c4ccc1[nH]4)C=C3)C=C2]

Embodiment 31 The method of any one of embodiments 1 to 30, wherein said method further comprises contacting the compound of formula (I) or formula (Ia) with a metal salt.

Embodiment 32 The method of embodiment 31, wherein said metal salt is a transition metal salt.

Embodiment 33 The method of embodiment 32, wherein said metal salt is a manganese salt.

Embodiment 34 A method for synthesizing a compound of formula

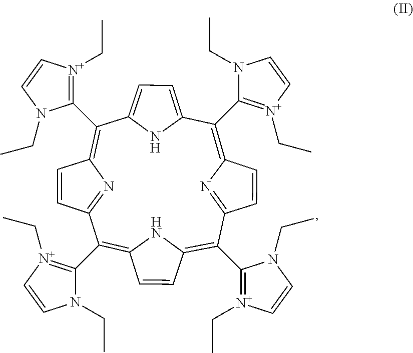 [SMILES: CCn1cc[n+](CC)c1-c1c2nc(c(-c3n(CC)cc[n+]3CC)c3ccc([nH]3)c(-c3n(CC)cc[n+]3CC)c3nc(c(-c4n(CC)cc[n+]4CC)c4ccc1[nH]4)C=C3)C=C2]

said method comprising: contacting with an ethylating agent a compound having the formula

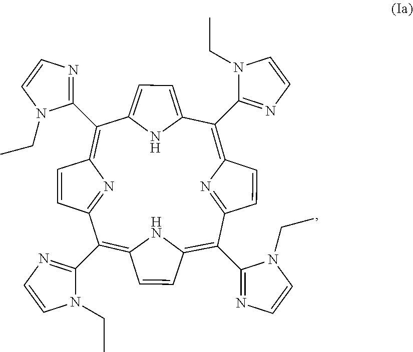 [SMILES: CCn1ccnc1-c1c2nc(c(-c3nccn3CC)c3ccc([nH]3)c(-c3nccn3CC)c3nc(c(-c4nccn4CC)c4ccc1[nH]4)C=C3)C=C2]

thereby synthesizing a compound of formula (II).

Embodiment 35 The method of embodiment 34, further comprising a counterion selected from the group consisting of a halogen anion, SCN⁻, HSO₄⁻, SO₄⁻², H₂PO₄⁻¹, HPO₄⁻², PO₄⁻³, NO₃⁻, PF₆⁻, or BF₄⁻.

Embodiment 36 The method of embodiment 34 or 35, wherein said method further comprises: (i) contacting about one equivalent of a pyrrole with about one equivalent of 1-ethyl-1H-imidazole-2-carbaldehyde, wherein said contacting is performed in a solvent comprising a positive azeotrope; (ii) allowing said pyrrole to react with said 1-ethyl-1H-imidazole-2-carbaldehyde, in said solvent under azeotropic distillation conditions, thereby forming a substituted-porphyrinogen; and (iii) oxidizing said substituted-porphyrinogen, thereby synthesizing a substituted porphyrin having formula (Ia).

Embodiment 37 The method of any one of embodiments 34 to 36, wherein said ethylating agent is alkyl-halogen.

Embodiment 38 The method of any one of embodiments 34 to 37, wherein said alkyl-halogen is iodoethane.

Embodiment 39 The method of any one of embodiments 34 to 37, wherein said contacting is performed at a temperature of about 100° C.

Embodiment 40 The method of any one of embodiments 34 to 36, wherein said ethylating agent is a Meerwein salt.

Embodiment 41 The method of embodiment 40, wherein said Meerwein salt is triethyloxonium tetrafluoroborate or triethyloxonium hexafluorophosphate.

Embodiment 42 The method of embodiment 40 or 41, wherein said contacting is performed at a temperature from about 50° C. to about 100° C.

Embodiment 43 The method of any one of embodiments 34 to 42, wherein said contacting is performed at a temperature of about 80° C.

Embodiment 44 The method of any one of embodiments 34 to 42, wherein said contacting is performed in dimethylformamide.

Embodiment 45 The method of any one of embodiments 34 to 44, wherein said method further comprises precipitation of the compound having formula (II) with a precipitating agent.

Embodiment 46 The method of embodiment 45, wherein said precipitating agent is an ammonium salt.

Embodiment 47 The method of any one of embodiments 34 to 46, wherein said method further includes contacting the compound of formula (II) with a metal salt.

Embodiment 48 The method of embodiment 47, wherein said metal salt is a transition metal salt.

Embodiment 49 The method of embodiment 47 or 48, wherein said metal salt is a manganese salt.

Embodiment 50 A method for synthesizing a hydrate compound having the formula

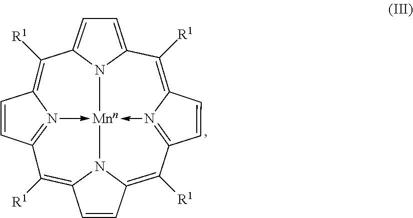

wherein R¹ is substituted or unsubstituted heterocycloalkyl or substituted or unsubstituted heteroaryl;

and n is 2 or 3, said method comprising: (i) contacting a compound of formula

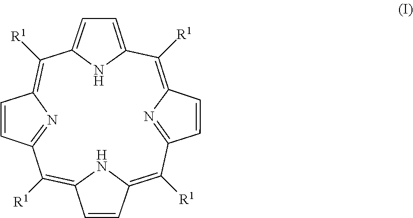

with over about 2 equivalents of a Mn(III) salt in a solvent, thereby forming a reaction mixture; (ii) heating said reaction mixture thereby synthesizing a compound of formula (III); and (iii) hydrating said compound of formula (III) thereby forming a hydrate of compound (III).

Embodiment 51 The method of embodiment 50, wherein R¹ is substituted or unsubstituted imidazolyl, substituted or unsubstituted pyrazolyl, substituted or unsubstituted thiazolyl, or substituted or unsubstituted triazolyl.

Embodiment 52 The method of embodiment 50 or 51, wherein R¹ is substituted imidazolyl.

Embodiment 53 The method of any one of embodiments 50 to 52, wherein R¹ is:

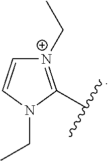 [SMILES: *c1n(CC)cc[n+]1CC]

Embodiment 54 The method of any one of embodiments 50 to 53, wherein R¹ is substituted or unsubstituted heteroaryl.

Embodiment 55 The method of any one of embodiments 50 to 54, wherein R¹ is substituted or unsubstituted pyridinyl, substituted or unsubstituted pyrazinyl, substituted or unsubstituted pyrimidinyl, or substituted or unsubstituted pyridazinyl.

Embodiment 56 The method of any one of embodiments 50 to 55, wherein n is 3.

Embodiment 57 The method of any one of embodiments 50 to 56, wherein said compound of formula (I) is contacted with about 2 to about 10 equivalents of Mn(III) salt.

Embodiment 58 The method of any one of embodiments 50 to 57, wherein said compound of formula (I) is contacted with about 2 to about 5 equivalents of Mn(III) salt.

Embodiment 59 The method of any one of embodiments 50 to 58, wherein said compound of formula (I) is contacted with about 2 to about 3 equivalents of Mn(III) salt.

Embodiment 60 The method of any one of embodiments 50 to 59, wherein said solvent is acetonitrile.

Embodiment 61 The method of any one of embodiments 50 to 60, wherein said reaction mixture is heated to a temperature of about 15° C. to about 70° C.

Embodiment 62 The method of any one of embodiments 50 to 61, wherein said method further comprises filtering said reaction mixture.

Embodiment 63 The method of any one of embodiments 50 to 62, wherein said method further comprises allowing said reaction mixture to cool to a temperature of about 10° C. to about 30° C.

Embodiment 64 The method of any one of embodiments 50 to 63, wherein said hydrating comprises contacting compound of formula (III) with a gas having a relative humidity from about 30% to about 70%.

Embodiment 65 The method of embodiment 64, wherein said compound of formula (III) is dried after contacting with said gas.

Embodiment 66 The method of any one of embodiments 50 to 65, wherein said method further comprises contacting said reaction mixture with an anion-exchanging agent and allowing said mixture to react with said anion-exchanging agent.

Embodiment 67 The method of synthesis of any one of embodiments 50 to 67, wherein the compound has the formula:

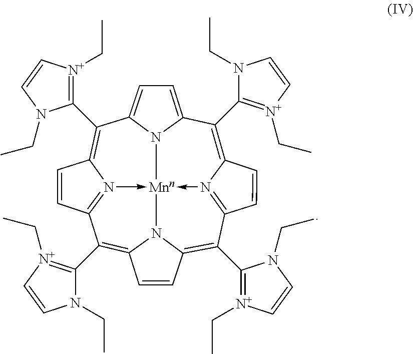

Embodiment 68 The method of embodiment 67, further comprising a counterion selected from the group consisting of a halogen anion, SCN⁻, HSO₄⁻, SO₄⁻², H₂PO₄⁻¹, HPO₄⁻², PO₄⁻³, NO₃⁻, PF₆⁻, or BF₄⁻.

Embodiment 69 The method of embodiment 68, wherein n is 3.

Embodiment 70 A container comprising a plurality compounds, wherein said plurality of compounds have the formula:

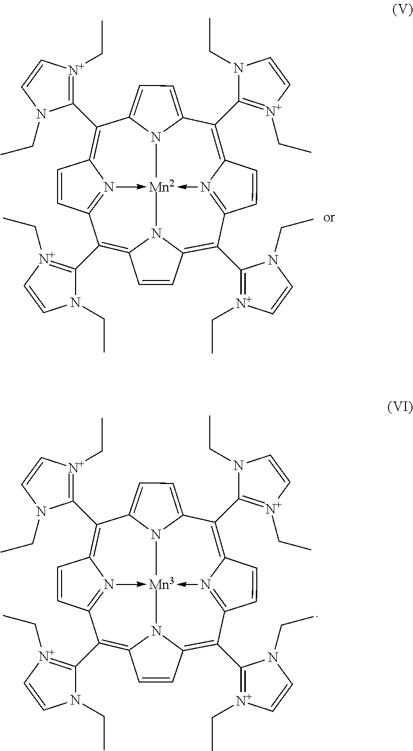

Embodiment 71 The container of embodiment 70, wherein at least 60% of said plurality of compounds have formula (VI).

Embodiment 72 The container of embodiment 70 or 71, wherein at least 90% of said plurality of compounds have formula (VI).

Embodiment 73 The container of embodiment 70 or 71, wherein at least 95% of said plurality of compounds have formula (VI).

Embodiment 74 The container of any one of embodiments 70 to 73, further comprising a counterion selected from the group consisting of a halogen anion, SCN⁻, HSO₄⁻, SO₄⁻², H₂PO₄⁻¹, HPO₄⁻², PO₄⁻³, NO₃⁻, PF₆⁻, or BF₄⁻.

Embodiment 75 The container of any one of embodiments 70 to 74, wherein said plurality of compounds is in water thereby forming a pharmaceutical formulation.

Embodiment 76 The container of embodiment 75, wherein said pharmaceutical formulation is at a pH of from about 3.5 to about 7.0.

Embodiment 77 The container of embodiment 75 or 76, wherein said pharmaceutical formulation consists essentially of water and the compound of embodiment 70.

Embodiment 78 The container of embodiment 75 or 76, wherein said pharmaceutical formulation consists of water, the compound of embodiment 70, and pH adjustment ions.

Embodiment 79 The container of embodiment 75 or 76, wherein the pharmaceutical formulation does not comprise a buffer.

Embodiment 80 The container of embodiment 75 or 76, wherein the pharmaceutical formulation does not comprise a pharmaceutical excipient.

Embodiment 81 A pharmaceutical formulation comprising water and a compound having the formula:

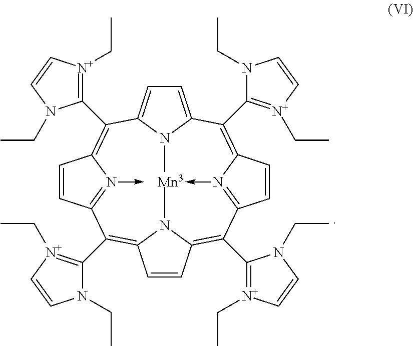

Embodiment 82 The pharmaceutical formulation of embodiment 81, wherein the formulation comprises less than 10% Mn(II).

Embodiment 83 The pharmaceutical formulation of embodiment 81 or 82, wherein the formulation comprises less than 5% Mn(II).

Embodiment 84 The pharmaceutical formulation of any one of embodiments 81 to 83, wherein the formulation comprises less than 1% Mn(II).

Embodiment 85 The pharmaceutical formulation of any one of embodiments 81 to 84, wherein said formulation has a pH of from about 3.5 to about 7.0.

Embodiment 86 The pharmaceutical formulation of embodiment 81 to 85 consisting essentially of water and said compound.

Embodiment 87 The pharmaceutical formulation of embodiment 81 to 85 consisting of water, the compound, and pH adjustment ions.

Embodiment 88 The pharmaceutical formulation of embodiment 81 to 85, wherein the pharmaceutical formulation does not comprise a buffer.

Embodiment 89 The pharmaceutical formulation of embodiment 81 to 85, wherein the pharmaceutical formulation does not comprise a pharmaceutical excipient.

Embodiment 90 A method for purifying a compound of formula:

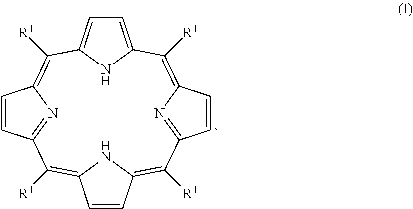

said method comprising: (i) combining a compound of formula (I) and a purification solvent in a reaction vessel thereby forming a purification mixture, wherein said compound is insoluble in said purification solvent; (ii) heating said purification mixture; (iii) cooling said purification mixture; and (iv) filtering said purification mixture thereby purifying a compound of formula (I).

Embodiment 91 The method of embodiment 90, wherein said purification solvent is 2-butanone, 1,4-dioxane, acetonitrile, ethyl acetate or cyclohexanone.

Embodiment 92 The method of embodiment 90 or 91, wherein said purification solvent is 2-butanone.

Embodiment 93 The method of any one of embodiments 90 to 92, wherein said purification mixture is heated to about 80° C.

Embodiment 94 The method of any one of embodiments 90 to 93, wherein said purification mixture is heated for about 1 hour.

Embodiment 95 The method of any one of embodiments 90 to 94, wherein said purification mixture is cooled to about 0° C.

Embodiment 96 The method of any one of embodiments 90 to 95, wherein said purification mixture is cooled for about 2 hours.

Embodiment 97 The method of any one of embodiments 90 to 96, wherein said filtering comprises washing the filter cake comprising said compound with a washing solvent.

Embodiment 98 The method of any one of embodiments 90 to 97, wherein said washing solvent comprises 2-butanone or tert-butyl methyl ether.

Embodiment 99 A method for purifying a compound having the formula:

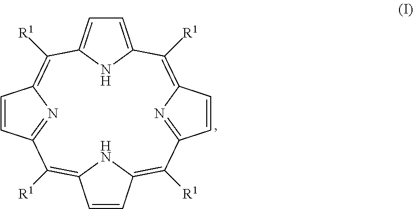

wherein, said method comprises: (i) dissolving a compound of formula (I) in a purifying solvent in a reaction vessel to form a purifying mixture; (ii) heating said purifying mixture; (iii) cooling said purifying mixture; (iv) drying said purifying mixture thereby purifying a compound of formula (I).

Embodiment 100 The method of embodiment 99, wherein said purifying solvent is dimethylformamide.

Embodiment 101 The method of embodiment 99 or 100, wherein said purifying mixture is heated to about 150° C.

Embodiment 102 The method of any one of embodiments 99 to 101, wherein said purifying mixture is heated for about 1 hour.

Embodiment 103 The method of any one of embodiments 99 to 102, wherein said purifying mixture is cooled to about 25° C.

Embodiment 104 The method of any one of embodiments 99 to 103, wherein said purifying mixture is filtered following cooling.

Embodiment 105 The method of any one of embodiments 99 to 104, wherein said filtering comprises washing the filter cake comprising said compound of formula (I) with dimethylformamide.

Embodiment 106 A crystal comprising a compound having the formula:

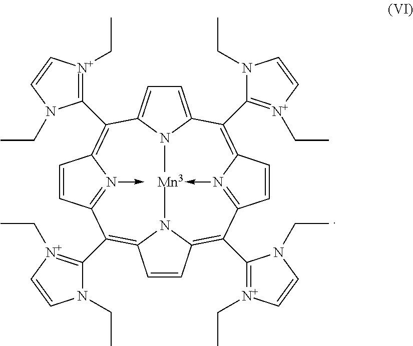

Embodiment 107 The crystal of embodiment 106, wherein the crystal is a hydrate.

Embodiment 108 A crystalline form of [5,10,15,20-tetrakis(1,3-diethylimidazolium-2-yl)porphyrinato]manganese(III) chloride hydrate complex characterized by an x-ray powder diffraction spectrum, said x-ray powder diffraction spectrum comprising angle 2θ peaks at about 6.9±0.2, 8.2±0.2, 9.5±0.2, 11.4±0.2, 12.8±0.2, 14.5±0.2, 15.0±0.2, 16.1±0.2, 16.3±0.2, 18.1±0.2, 20.3±0.2, 23.5±0.2, 24.8±0.2, 25.6±0.2, 26.5±0.2, and 29.2±0.2, wherein said an x-ray powder diffraction spectrum is obtained using a Cu Kα radiation source (1.54 Å).

Embodiment 109 The crystalline form of 108, wherein said x-ray powder diffraction spectrum further comprises angle 2θ peaks at about 13.8±0.2, 17.4±0.2, 19.0±0.2, 19.4±0.2, 20.7±0.2, 21.1±0.2, 21.5±0.2, 22.0±0.2, 22.5±0.2, 22.8±0.2, 26.9±0.2, 27.6±0.2, 28.5±0.2, 30.2±0.2, 30.5±0.2, 31.2±0.2, 37.3±0.2, 38.5±0.2, and 41.1±0.2.

Embodiment 110 A crystalline form of [5,10,15,20-tetrakis(1,3-diethylimidazolium-2-yl)porphyrinato]manganese(III) chloride hydrate complex characterized by an x-ray powder diffraction spectrum, said x-ray powder diffraction spectrum comprising d spacings at about 12.85, 10.82, 9.28, 7.78, 6.91, 6.11, 5.91, 5.49, 5.42, 4.89, 4.37, 3.78, 3.58, 3.47, 3.36, and 3.06, wherein said an x-ray powder diffraction spectrum is obtained using a Cu Kα radiation source (1.54 Å).

Embodiment 111 The crystalline form of embodiment 110, wherein said x-ray powder diffraction spectrum further comprises d spacings at about, 7.57, 6.44, 5.10, 4.67, 4.58, 4.29, 4.2, 4.13, 4.05, 3.96, 3.89, 3.31, 3.22, 3.13, 2.96, 2.93, 2.86, 2.41, 2.34, and 2.19.

Embodiment 112 A crystalline form of [5,10,15,20-tetrakis(1,3-diethylimidazolium-2-yl)porphyrinato]manganese(III) chloride hydrate complex characterized by an x-ray powder diffraction spectrum, said x-ray powder diffraction spectrum comprising angle 2θ peaks at about 26.2±0.2, 22.9±0.2, 20.0±0.2, 18.6±0.2, 15.2±0.2, 13.7±0.2, 13.5±0.2, 13.0±0.2, 12.4±0.2, 11.4±0.2, 10.6±0.2, 8.9±0.2, 6.8±0.2, and 6.0±0.2, wherein said an x-ray powder diffraction spectrum is obtained using a Cu Kα radiation source (1.54 Å).

Embodiment 113 The crystalline form of 112, wherein said x-ray powder diffraction spectrum further comprises angle 2θ peaks at about 29.4±0.2, 28.5±0.2, 27.5±0.2, 27.0±0.2, 25.7±0.2, 25.2±0.2, 23.7±0.2, 17.8±0.2, 17.1±0.2, 14.6±0.2, 10.9±0.2, 9.9±0.2, and 8.2±0.2.

Embodiment 114 A crystalline form of [5,10,15,20-tetrakis(1,3-diethylimidazolium-2-yl)porphyrinato]manganese(III) chloride hydrate complex characterized by an x-ray powder diffraction spectrum, said x-ray powder diffraction spectrum comprising d spacings at about 14.74, 12.93, 9.99, 8.34, 7.74, 7.14, 6.80, 6.55, 6.45, 5.83, 4.78, 4.43, 3.89, and 3.40, wherein said an x-ray powder diffraction spectrum is obtained using a Cu Kα radiation source (1.54 Å).

Embodiment 115 The crystalline form of embodiment 114, wherein said x-ray powder diffraction spectrum further comprises d spacings at about 10.82, 8.90, 8.10, 6.05, 5.19, 4.98, 3.75, 3.54, 3.47, 3.30, 3.24, 3.13, and 3.04.

Embodiment 116 A crystalline form of [5,10,15,20-tetrakis(1,3-diethylimidazolium-2-yl)porphyrinato]manganese(III) chloride hydrate complex characterized by an x-ray powder diffraction spectrum, said x-ray powder diffraction spectrum comprising angle 2θ peaks at about 27.7±0.2, 26.6±0.2, 19.9±0.2, 15.4±0.2, 14.7±0.2, 11.6±0.2, 10.1±0.2, 8.6±0.2, and 6.9±0.2, wherein said an x-ray powder diffraction spectrum is obtained using a Cu Kα radiation source (1.54 Å).

Embodiment 117 The crystalline form of 116, wherein said x-ray powder diffraction spectrum further comprises angle 2θ peaks at about 29.6±0.2, 25.7±0.2, 23.4±0.2, 20.4±0.2, and 13.7±0.2.

Embodiment 118 A crystalline form of [5,10,15,20-tetrakis(1,3-diethylimidazolium-2-yl)porphyrinato]manganese(III) chloride hydrate complex characterized by an x-ray powder diffraction spectrum, said x-ray powder diffraction spectrum comprising d spacings at about 12.89, 10.27, 8.79, 7.60, 6.04, 5.74, 4.45, 3.35, and 3.22, wherein said an x-ray powder diffraction spectrum is obtained using a Cu Kα radiation source (1.54 Å).

Embodiment 119 The crystalline form of embodiment 118, wherein said x-ray powder diffraction spectrum further comprises d spacings at about 6.45, 4.35, 3.80, 3.46, and 3.02.

Embodiment 120 A crystalline form of [5,10,15,20-tetrakis(1,3-diethylimidazolium-2-yl)porphyrinato]manganese(III) chloride hydrate complex characterized by an x-ray powder diffraction spectrum, said x-ray powder diffraction spectrum comprising angle 2θ peaks at about 29.5±0.2, 27.3±0.2, 26.3±0.2, 24.7±0.2, 23.5±0.2, 22.5±0.2, 21.6±0.2, 20.5±0.2, 19.3±0.2, 17.7±0.2, 13.1±0.2, 10.8±0.2, 9.9±0.2, 8.5±0.2, and 6.0±0.2, wherein said an x-ray powder diffraction spectrum is obtained using a Cu Kα radiation source (1.54 Å).

Embodiment 121 The crystalline form of 120, wherein said x-ray powder diffraction spectrum further comprises angle 2θ peaks at about 32.6±0.2, 19.8±0.2, 18.6±0.2, and 14.8±0.2.

Embodiment 122 A crystalline form of [5,10,15,20-tetrakis(1,3-diethylimidazolium-2-yl)porphyrinato]manganese(III) chloride hydrate complex characterized by an x-ray powder diffraction spectrum, said x-ray powder diffraction spectrum comprising angle 2θ peaks at about 23.5±0.2, 9.1±0.2, 6.9±0.2, and 5.8±0.2, wherein said an x-ray powder diffraction spectrum is obtained using a Cu Kα radiation source (1.54 Å).

Embodiment 123 The crystalline form of 122, wherein said x-ray powder diffraction spectrum further comprises angle 2θ peaks at about 27.5±0.2, 24.6±0.2, 18.2±0.2, 13.9±0.2, 13.0±0.2, 11.7±0.2, and 7.9±0.2.

Embodiment 124 A crystalline form of [5,10,15,20-tetrakis(1,3-diethylimidazolium-2-yl)porphyrinato]manganese(III) chloride hydrate complex characterized by an x-ray powder diffraction spectrum, said x-ray powder diffraction spectrum comprising d spacings at about 15.12, 12.74, 9.75, and 3.78, wherein said an x-ray powder diffraction spectrum is obtained using a Cu Kα radiation source (1.54 Å).

Embodiment 125 The crystalline form of embodiment 124, wherein said x-ray powder diffraction spectrum further comprises d spacings at about 11.14, 7.55, 6.81, 6.36, 4.87, 3.62, and 3.24.

Embodiment 126 A crystalline form of [5,10,15,20-tetrakis(1,3-diethylimidazolium-2-yl)porphyrinato]manganese(III) chloride hydrate complex characterized by an x-ray powder diffraction spectrum, said x-ray powder diffraction spectrum comprising angle 2θ peaks at about 27.7±0.2, 23.6±0.2, 23.1±0.2, 20.7±0.2, 6.9±0.2, and 5.8±0.2, wherein said an x-ray powder diffraction spectrum is obtained using a Cu Kα radiation source (1.54 Å).

Embodiment 127 The crystalline form of 126, wherein said x-ray powder diffraction spectrum further comprises angle 2θ peaks at about 29.2±0.2, 28.9±0.2, 27.1±0.2, 26.5±0.2, 26.2±0.2, 24.8±0.2, 22.4±0.2, 22.2±0.2, 21.5±0.2, 20.3±0.2, 18.1±0.2, 17.3±0.2, 16.3±0.2, 14.9±0.2, 13.8±0.2, 11.5±0.2, and 9.2±0.2.

Embodiment 128 A crystalline form of [5,10,15,20-tetrakis(1,3-diethylimidazolium-2-yl)porphyrinato]manganese(III) chloride hydrate complex characterized by an x-ray powder diffraction spectrum, said x-ray powder diffraction spectrum comprising angle 2θ peaks at about 27.7±0.2, 20.7±0.2, 13.8±0.2, 11.4±0.2, 9.5±0.2, 8.2±0.2, and 6.9±0.2, wherein said an x-ray powder diffraction spectrum is obtained using a Cu Kα radiation source (1.54 Å).

Embodiment 129 The crystalline form of 128, wherein said x-ray powder diffraction spectrum further comprises angle 2θ peaks at about 23.5±0.2, 22.8±0.2, 16.3±0.2, and 5.9±0.2.

Embodiment 130 A crystalline form of [5,10,15,20-tetrakis(1,3-diethylimidazolium-2-yl)porphyrinato]manganese(III) chloride hydrate complex characterized by an x-ray powder diffraction spectrum, said x-ray powder diffraction spectrum comprising d spacings at about 12.84, 10.83, 9.26, 7.77, 6.43, 4.29, and 3.22, wherein said an x-ray powder diffraction spectrum is obtained using a Cu Kα radiation source (1.54 Å).

Embodiment 131 The crystalline form of embodiment 130, wherein said x-ray powder diffraction spectrum further comprises d spacings at about 15.07, 12.84, 10.83, 9.26, 7.77, 6.43, 5.42, 4.29, 3.89, 3.79, and 3.22.

### VII. EXAMPLES

### Example 1

Instruments and Equipment. HPLC: Agilent 1100 system equipped with gradient capability, column temperature control, UV detector and electronic data collection and processing system, or equivalent. Columns: Ace 3 C8 3 micron particle size; Supelco RP-Amide 3 micron particle size and PHENOMENEX® KINETIX® XBC18 100A, 2.6 micron particle size, all column dimensions 150×4.6 mm. Autosampler capable of 10 μL injection. Analytical balance capable of weighing to ±0.1 mg. Class A volumetric flasks and pipettes. NMR: Bruker NMR Automation AVANCE™ 300, NMR tubes 5 mm×7″ catalog #NE-HL5-7 from New Era Enterprises or equivalent. Deuterated solvent from Cambridge Isotope Laboratories such as chloroform d1, DMSO-d6, and methanol-d4 were used for sample dissolution. XRPD: X-ray powder diffraction patterns were obtained using a Bruker D8 Advance equipped with a Cu Kα radiation source (1.54° A), a 9-position sample holder and a LYNXEYE Super Speed Detector. Samples were placed on zero-background, silicon plate holders.

Reagents and Materials. Bulk solvents: acetone, acetonitrile, methanol, toluene, DCM, TBME, ethyl acetate, MEK, DMF. HPLC solvents were obtained from OMNISLOV®. HPLC water was used from MILLI-Q® system. Deuterated solvents were obtained from Cambridge Isotope Laboratories, Inc. Reagents that were purchased from Alfa Aesar: pyrrole, propionic acid, anhydrous DMF, ethyl iodide. Reagents purchased from Sigma Aldrich: ammonium hexafluorophosphate, tetrabutylammonium chloride (≧97.% (AT). Manganese(III) acetate dihydrate was purchased either from Acros or Sigma Aldrich. 1-ethyl-1H-imidazole-2-carbaldehyde was prepared in-house. Preparative thin layer chromatography was carried out using ANALTECH® silica GF plates.

Synthesis of porphyrin rings The first synthesis step for formula (I) is based on the Adler and Longo modification of the Rothemund porphyrin synthesis, which uses propionic acid at reflux temperature (141° C.) as a solvent. The reaction is fast and the maximum yield of formula (I) is achieved in just a few minutes. Further heating causes significant yield decrease and the formation of poorly identifiable polymerization products. Bearing in mind the extended heating and cooling times associated with large volumes, the application of traditional batch technology may be problematic.

General formation of a porphyrin in the Rothemund reaction proceeds in two major steps. First, formation of porphyrinogen, is a reversible process, which is accompanied by the formation of four molecules of water. Removal of water by adding water soluble salts or by azeotropic water distillation may shift the equilibrium and improve the yield. Similarly, the oxidation of the porphyrinogen to the final porphyrin may shift the equilibrium and increase the yield.

Using equilibrium shift techniques may involve either adding or removing the reaction components while the reaction of interest is still in progress. This approach is not compatible with PFR techniques. Neither introduction of oxygen (to convert the porphyrinogen to formula (I)) nor removal of water (to prevent the ring-opening processes) can be made without ready access to the reaction mixture.

The porphyrinogen intermediate can be oxidized to the porphyrin product by a number of oxidants, including air. In these studies the yield the porphyrin does not depend on whether or not the initial reaction mixture was exposed to air. This observation can be explained by either oxidation of porphyrinogen by other reaction products or by its oxidation during workup/handling. Even though the HPLC data indicated immediate porphyrin formation even without oxygen, this observation could be explained by the oxidation during subsequent analysis.

Whether the absence of oxygen in the isolation step prevents porphyrin formation was investigated. The Rothemund reaction was performed under nitrogen blanket and demonstrated complete oxidation to the porphyrin. The reaction was investigated in the batch mode. Reasonably volatile carboxylic acids either neat or as a mixture with other solvents, were initially evaluated at room temperature.

TABLE 2.1
Time required for achieving maximum yield of
porphyrin at room temperature in neat carboxylic acids.

| Carboxylic acid | Time (h) | Maximum yield |
|---|---|---|
| Formic | 17 | 4.8 |
| Acetic | 96 | 6.0 |
| Propionic | 38 | 6.0 |

Several diluents, most of which form low-boiling azeotropes with water, were also tested in 1:1 v/v mixtures with propionic acid at room temperature. The yields were determined at 60 h and are given in Table 2.2. The results above indicate that the preparation of the porphyrin can be achieved in batch mode even at room temperature.

TABLE 2.2
Influence of solvent additives on yield of porphyrin
in propionic acid at room temperature at 60 h.

| Solvent additive | Yield (%) | Solvent additive | Yield (%) |
|---|---|---|---|
| No (control) | 5.8 | Dimethylformamide | 1.8 |
| Dichloromethane | 1.5 | Chloroform | 1.5 |
| Tetrahydrofuran | 1 | Tetrachloroethylene | 0.2 |
| t-BuMe ester | 1.1 | 1,1,2,2-tetrachloroethane | 1.6 |
| Ethyl Acetate | 1.5 | Acetonitrile | 1.3 |

Performing the reaction at elevated temperature improves the yield and accelerates the condensation process when using acetic and propionic acids. This effect is more pronounced for acetic acid.

Performing the condensation reaction at temperatures close to a 100° C. required higher boiling cosolvents for azeotropic water removal. The azeotropic removal of water surprisingly significantly improved the yield when using chlorobenzene, m-xylene or toluene as cosolvents. This effect was most pronounced in propionic acid-toluene where a solution yield of 23% was achieved in 40 h.

Successful application of the azeotropic distillation technique to the preparation of porphyrins by the Rothemund method prompted use of the same approach for the condensation between aldehydes and dipyrromethane. The yields achieved for the latter reactions (21%) were nearly identical to the yields obtained for the Rothemund condensation. The leveling effect of the azeotropic water removal was attributed to the reduced number of water molecules formed in the condensation (i.e. two molecules) as compared to four water molecules abstracted in the standard Rothemund condensation. The reduced amount of water formed in the condensation makes the water removal process less influencing.

Using catalytic amounts of p-chloranil and iron phthalocyanin with air (as the stoichiometric oxidant) or stoichiometric oxidants such as DDQ or m-C PBA did not appreciably shift the equilibrium of the Rothemund reaction by oxidizing porphyrinogen and resulted substantially the same yields without their use. Importantly, the same yield was observed when the condensation reaction was performed under nitrogen followed by room temperature air oxidation. Thus the oxidation of the porphyrinogen intermediate proceeds during the reaction despite the absence of oxygen. This observation allows for a safer execution of the synthesis on large scale and eliminates the heating of flammable solvents at elevated temperatures in the presence of oxygen.

An additional yield-improving method, slow addition of the reagents, stems from the observation that higher yields of formula (I) obtained in more dilute systems. Slow addition of the reaction components to the refluxing reaction mixture effectively results in performing the reaction at lower concentration at any time point of the reagent addition. Only when the starting materials are added completely does the concentration of the reaction mixture reach its expected value. At all previous points the concentration is lower and would be expected to give a higher yield when compared to the scenario of having all the components added at once.

Addition of pyrrole and aldehyde over 10 hours to a refluxing mixture of propionic acid and toluene was accompanied by azeotropic water removal. The yields of the reaction were universally higher than for immediate addition of the reagents and reached yield value of 31% in 48 hours.

Apart from residual solvent peaks and minor impurities the NMR data indicates that air oxidation is not necessary and compounds of formula (I) can be synthesized and isolated under nitrogen. Thin layer chromatography exhibited same major spots for samples prepared under nitrogen and in air.

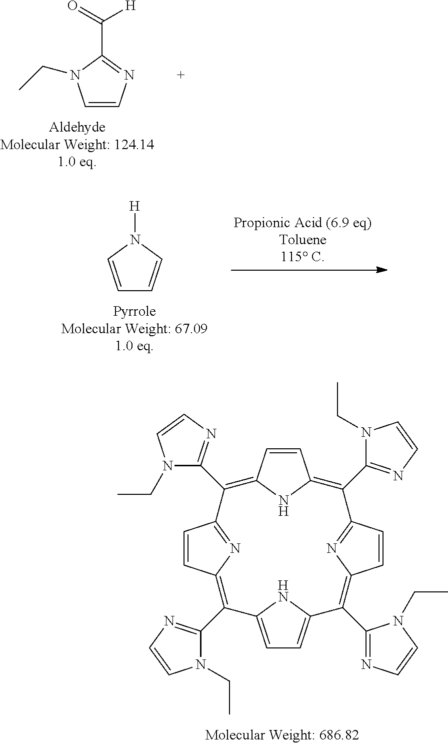

To a 72 L round bottom flask equipped with a Dean-Stark trap, condenser, nitrogen inlet, thermocouple, and an overhead stirrer in a heating mantle was charged toluene (21.8 kg) and propionic acid (14.5 kg). The mixture was heated until a steady reflux was reached (112° C.). Pyrrole (1892 g, 28.2 mol, 1832.2 g purchased from Sigma-Aldrich, 60.0 g purchased from Alfa Aesar) and 1-ethylimidazole-2-carboxyaldehyde (3500 g. 28.2 mol) were added in 10 approximately equal portions over 9 hours (one charge per hour of each using 2 addition funnels, charged simultaneously). After the addition of the reagents was complete, the reaction mixture was stirred for an additional 15 hours at reflux (684 g of water was collected in the Dean-Stark trap) before being slowly cooled to room temperature. A sample of the reaction mixture was removed for HPLC analysis. The solution yield was determined as 14.6% (wt/wt). To the reaction mixture was added purified water (57 kg) and the mixture was transferred to a 100 L jacketed reactor. The mixture was stirred for 25 minutes before allowing the layers to separate. The layers were separated and the organic layer was washed with 2116 g of 10% propionic acid in water. The aqueous layers were combined (80.26 kg) in a 100 L jacketed reactor and a sample was removed for HPLC analysis. The solution yield after aqueous work up was determined to be 13.8% (wt/wt). The combined aqueous layers were cooled to 8° C. and then basified with a 40% sodium hydroxide solution (16.4 kg) to pH 11.1 while keeping the batch below 20° C. To minimize issues with unwanted tar formation, the batch should be kept below 10° C., as the solids become more difficult to work with as the batch warms up.

The resulting suspension was cooled to 4° C. and filtered by vacuum filtration (5 μm Nylon filter cloth was used on an 18″ Polyethylene filter) in portions. The portions were collected and kept below 5° C. until the filtration was complete. The solids were slurried and washed with water (23.4 kg total, ˜5° C.). The solids were transferred to drying trays which were kept under nitrogen for 66 hours 15 minutes. Drying under vacuum at 60±5° C. afforded 4.45 kg of a black solid. The solid was analyzed for residual solvent by loss on drying. The solids were determined to contain 2.40% solvent (Target≦15%).

MEK Purification. To a 100 L jacketed reactor was charged the crude porphyrin (4.41 kg) and 2-butanone (37.0 kg 10 volumes based on crude weight). The mixture was heated to reflux (80° C.) and was held for 1 hour 15 minutes. The batch was cooled slowly overnight to 0° C. and held for over 10 hours 20 minutes. The resulting suspension was filtered by vacuum filtration (5 μm Nylon filter cloth was used on an 18″ Polyethylene filter) over 1 hour 50 minutes. The filtered solids were washed with 2-butanone (5.7 kg, ˜5° C.), followed by tert-butyl methyl ether (7.9 kg, room temperature). The solids were dried under nitrogen for 30 minutes. Drying under high vacuum at 70±5° C. afforded 1.23 kg of a brown solid. A sample of the solids was taken for HPLC and was determined to contain 566 g (46.0% wt/wt) of porphyrin.

DMF Recrystallization. To a 50 L round bottom flask equipped with condenser, nitrogen inlet, thermocouple and an overhead stirrer in a heating mantle was charged semi pure porphyrin and dimethylformamide (17.1 kg). The slurry was heated to 153° C. and held for 90 minutes before slowly cooling over 17 hours 25 minutes to 18° C. The slurry was filtered by vacuum filtration (5 μm Nylon filter cloth was used on an 18″ Polyethylene filter) over 17 minutes. The filter cake was washed with dimethylformamide (5.6 kg, room temperature) and tert-butyl methyl ether (7.9 kg, room temperature). The solids were dried on the filter under vacuum and nitrogen for 66 hours 10 minutes. Drying under high vacuum at 70±5° C. afforded 709.1 g of a dark red powder. A sample of the solids was taken for HPLC and was determined to contain 584 g (82.4% wt/wt) of porphyrin. Analysis of the porphyrin performed using 1H NMR determined the solids contained sodium propionate salt.

Water Slurry to Remove Sodium Propionate. To a vacuum filter (5 μm Nylon filter cloth was used on an 18″ Polyethylene filter) equipped with a nitrogen blanket was charged semi-pure porphyrin (709 g) and water (7090 g, 10 volumes). The slurry was stirred manually for 10 minutes at room temperature and then was filtered by vacuum filtration. The filter cake was washed with water (5×700 g, room temperature). Drying under high vacuum at 70±5° C. afforded 609.1 g of a purple powder. Analysis by HPLC determined the solids to be 94.4% porphyrin (wt/wt). A potency check performed using 1H NMR determined the solids to contain <1.0% sodium propionate salt.

### Example 2

Synthesis of alkylated porphyrins. Ethylation of formula (I) with triethyloxonium tetrafluoroborate (Meerwein salt) was investigated to streamline future required anion exchanges in the conversion of formula (II) to formula (III). The use of Meerwein salt also obviates the use of volatile genotoxic iodoethane.

Four different non-nucleophilic solvents (dichloromethane, tert-butyl methyl ether, acetonitrile and dimethylformamide) were tested at room temperature as the reaction media. While no conversion was observed in dichloromethane, dimethylformamide and tert-butyl methyl ether, acetonitrile resulted in a nearly quantitative conversion. Formation of the desired product (80% AUC) was, however, accompanied by two impurities with relative retention times identical to the impurities observed in the iodoethane ethylation. The level of these impurities was, however higher than in the traditional iodoethane method.

Different approaches were used to isolate the pure product:
1) Precipitation of the alkylated product as tetrachloride salt by addition of tetrabutylammonium chloride in acetonitrile. Even though the anion exchange yield was good, no upgrade in purity was observed.
2) Precipitation of the alkylated product as hexafluorophosphate salt by addition of ammonium hexafluorophosphate in methanol followed by various trituration or reprecipitation protocols. The purity of the resulting precipitates were monitored and gave 8.6; 8.0 and 9.4 minutes retention times for the desired product and two major impurities respectively. Product, isolated from dimethylformamide, exhibited the highest (96%) AUC purity, prompting another attempt to perform preparation the compound of formula (III) directly in dimethylformamide.

Synthesis of Porphyrin.

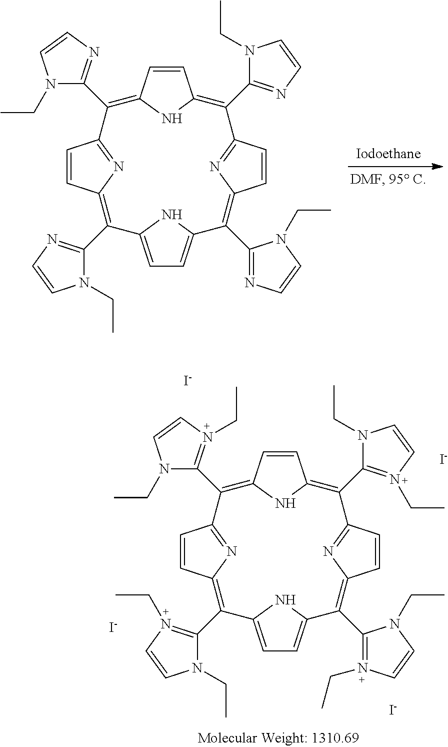

To a 100 L jacketed reactor equipped with a condenser, nitrogen inlet, and a thermocouple was charged porphyrin (1.021 kg) and dimethylformamide (27 kg). The mixture was heated to 102° C. and nitrogen was bubbled through the mixture to degas for 1 hour. Following degassing, the mixture was cooled to 100° C. and degassed (flask evacuated and nitrogen purged three times) iodoethane (7.31 kg, purchased from Alfa Aesar) was added. The reaction was held at 95±5° C. for 4 hours before being cooled overnight to room temperature. Ethyl acetate (65 kg) was added to the reaction and the slurry was stirred for 2 hours 30 minutes before being filtered by vacuum filtration (5 μm nylon filter cloth used on an 18″ polyethylene filter). The filter cake was washed with ethyl acetate (12 kg) and tert-butyl methyl ether (4.2 kg). Drying on the filter for 5 hours yielded 1.85 kg of a black powder.

To the 100 L reactor was charged crude porphyrin (1.84 kg) and dimethylformamide (21 kg). The mixture was heated to 78° C. and ethyl acetate (30 kg) was added slowly, keeping the batch temperature above 70° C. The batch was then cooled overnight to room temperature before being filtered by vacuum filtration (5 μm nylon filter cloth used on an 18″ polyethylene filter). The filter cake was washed with ethyl acetate (2×4.1 kg) and tert-butyl methyl ether (1.7 kg). Drying under high vacuum at 60±5° C. afforded 1423.0 g of a dark purple powder. Analysis of the solids by HPLC determined the solids to contain 1329 g target compound (93.4% wt/wt) with a purity of 96.2% (AUC). Analysis by 1H NMR determined that the solids contain 6.3 wt % residual DMF.

The ethylation reaction was performed with Meerwein salt in dimethylformamide at 80° C. Since starting purity of the crude alkylation was found to be higher for reaction in dimethylformamide as compared to acetonitrile and the use of dimethylformamide obviates the solvent swap after the isolation of porphyrin (I)/CELITE® mixture, the subsequent process development was planned for the reaction in dimethylformamide.

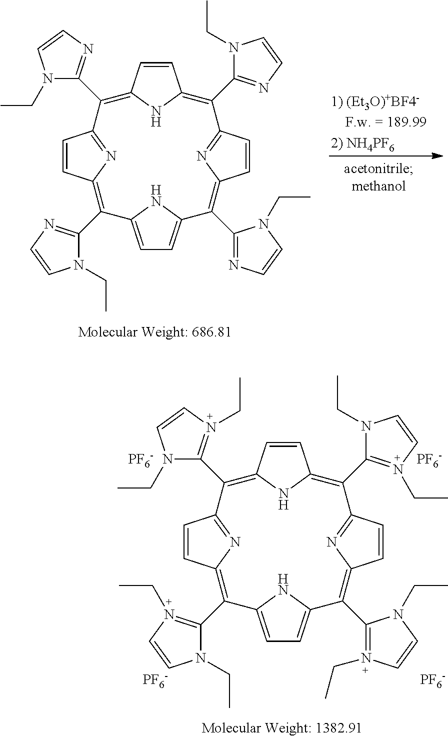

Starting material (1.0 g 1.46 mmol) was suspended in 10 ml of anhydrous acetonitrile. Triethyloxonium tetrafluoroborate (1.2 g, 6.32 mmol, 1.1-fold excess) was added as a solid and the reaction mixture was stirred at room temperature for 2 h. Filtered ˜10% solution of ammonium hexafluorophosphate in methanol (30 ml) was added at once and the reaction mixture was stirred for 15 minutes and filtered. The resulting cake was washed with methanol (5 ml) and tert-butyl methyl ether (10 ml).

### Example 3

Manganese Titrations. Two factors—excess of manganese(III) acetate and the reaction temperature influence the Mn(II) to Mn(III) ratio in the product. Higher reaction temperature facilitates reduction of Mn(III) to Mn(II) by the solvent. Excess of manganese(III) acetate plays an opposite role by reoxidizing Mn(II) to Mn(III). To test whether higher equivalents of Mn(II) increase the Mn(III) yield, experiments were performed using excess Mn(III) salts.

In order to have Mn(III) form as dominant form in the product the excess of Mn(OAc)₃-2H₂O was increased. The number of equivalents needed was decided based on two parameters: stability to reoxidation (i.e. no change in the UV-vis profile upon air exposure indicates the absence of Mn(II) form) and manganese content by elemental analysis.

The experiments were performed with 10, 5 and 3 equivalents of Mn(OAc)₃-2H₂O at 65° C. and showed no or minimal reoxidation indicating minimal presence of Mn(II) form. Based on these observation a procedure utilizing 3-fold excess of Mn(OAc)₃-2H₂O at 65° C. was repeatedly tested and resulted in no or minimal reoxidation stability and high Mn content.

To decrease the unwanted reduction to Mn(II) the reaction temperature was lowered which lessened the manganese reduction. The conversion to formula (III) proceeded even at 15° C. At 40° C. the reaction rate was acceptable and the resulting product contained limited amount of Mn(II). Incorporation of an additional 4 hours, 40° C. heating period further reduced the Mn(II) content. This heating period can be extended up to at least 80 hours with no adverse effects. As an additional measure the temperature of the product precipitation with tetrabutylammonium chloride was changed. The purpose of hot precipitation was to provide better crystallinity and better filterability for the Mn(III) product. The slow cooling of the reaction mixture in the presence of soluble manganese (III) acetate may potentially result in manganese coprecipitation.

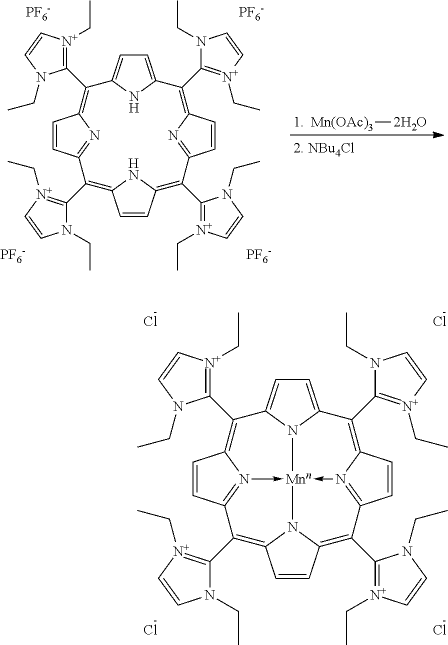

To a 50 L round bottom flask equipped with a nitrogen inlet and overhead stirring was charged the intermediate hexafluorophosphate salt (460.2 g) along with acetonitrile (16.1 kg). The mixture was stirred for 10 minutes to ensure complete dissolution and filtered through a 0.22 μm filter into a clean 50 L round bottom flask in a heating mantle. Rinsed forward with acetonitrile (1.5 kg) and the resulting solution was heated to 65±5° C. Manganese(III) acetate, dihydrate (270.7 g) was charged to the reactor and the reaction mixture stirred for 2 hours and 9 minutes before slowly cooling overnight to room temperature. The reaction mixture was filtered through a 0.22 μm filter into a clean 50 L flask in a heating mantle, rinsed forward with acetonitrile (1.55 kg), and heated again to 65±5° C. A solution of tetrabutylammonium chloride (1.405 kg) in acetonitrile (6.9 kg) was charged to the reactor over 25 minutes (temperature range during the charge: 57-62° C.). The reaction mixture was cooled slowly overnight, and the resulting slurry was filtered (filtration time was 25 minutes). The filter cake was washed with acetone (2×5.5 kg) and dried under nitrogen for 2 hours before placing in the vacuum oven to dry under full vacuum at room temperature. The solids were sampled periodically for GC and HPLC during the vacuum drying to monitor solvent and purity levels.

A sample of the batch was taken during the drying process to analyze by UV-Vis. The sample was dissolved in a solution of 0.1% TFA in water and immediately analyzed. The sample was left untouched for 30 minutes and reanalyzed. The UV-Vis profiles are unchanged over the 30 minute hold which indicates the absence of Mn(II) in the sample. Drying afforded 374.4 g (102% Yield) of a dark purple solid. The solids were passed through a 1 mm sieve.

Anion Exchange. To a 100 L jacketed reactor equipped with a reflux condenser, nitrogen inlet, thermocouple, and overhead stirring, was charged alkyl-porphyrin (II) (800.7 g) and methanol (31.8 kg). The mixture was heated to 55° C. and held for 47 minutes to ensure complete dissolution. A solution of ammonium hexafluorophosphate (1194 g) in methanol (10.7 kg) was prepared and charged to the reaction mixture through a 0.22 μm filter over a period of 32 minutes (temperature range during the charge was 54 to 60° C.). When the addition was complete, the reaction mixture was cooled slowly to room temperature overnight. The resulting slurry was filtered (3-5 μm Polypropylene filter cloth, filtration time: 28 minutes) and washed twice with methanol (3.3 kg each). The solids were dried under nitrogen for 3 hours 10 minutes before being placed into the vacuum oven to dry at 65±5° C. Drying afforded 767.9 g (91% yield) of a dark purple solid. HPLC purity: 98.0% AUC.

To a 100 L jacketed reactor equipped with a nitrogen inlet, thermocouple, and overhead stirring was charged alkyl-porphyrin (II) as a hexafluorophosphate salt (763.8 g), acetonitrile (19.4 kg), and manganese (III) acetate dihydrate (301.8 g) (as two equivalents). The reaction mixture was heated to 40° C. and monitored by HPLC for reaction completion. After 4 hours 5 minutes, the reaction was deemed complete (alkyl-porphyrin hexafluorophosphate was not detected). The reaction was stirred for an additional 4 hours at 40° C. before cooling slowly to ambient temperature overnight (˜13 hours). A sample of the cooled reaction mixture was removed to test the Mn(III)/Mn(II) ratio.

The mixture was filtered (18″ polyethylene filter, 3-8 μm polypropylene filter cloth) to remove excess manganese (III) acetate dihdyrate. The 100 L reactor was cleaned and the product solution was transferred back to the reactor, passing the solution through a 0.22 μm filter capsule. A solution of tetrabutyl ammonium chloride (2.30 kg) in acetonitrile (6.05 kg) was polish filtered into the reaction mixture over a period of 5 minutes at 21° C. The resulting slurry was stirred for 30 minutes at 21 to 22° C. and then filtered (18″ polyethylene filter, 3-8 μm polypropylene cloth, filtration time: 27 minutes). The filter cake was washed twice with acetone (1.5 kg each) and dried on the filter funnel under vacuum and nitrogen for 24 hours 15 minutes. Using a relative humidity generator, the humidity of the nitrogen flow was adjusted to 60% relative humidity and the drying continued (at this time the vacuum was turned off and drying continued only under the flow of nitrogen). Samples were removed periodically to test for % moisture (KF), XRPD, and residual solvents by GC. A sample of the solid was also removed to test the Mn(III)/Mn(II) ratio. Hydration was stopped after sample #4 (93 hours) as XRPD shows predominantly Form I. Hydration afforded 709.9 g (107% yield “corrected for water”) of a brown solid.

Hydration. 0.5 g of the compound of formula (III) was placed into a crystallizing dish open to ambient air (measured relative humidity 45-50%) for one hour 30 minutes then placed back into the vacuum oven to dry overnight. A sample was taken for GC after overnight drying. See Table 3.1 for results.

TABLE 3.1
Experiment Results

| | | | | Dimethyl- | HPLC |
|---|---|---|---|---|---|
| | Acetonitrile | Methanol | Acetone | formamide | Purity |
| Sample | (ppm) | (ppm) | (ppm) | (ppm) | (% AUC) |
| Cmpd | 123 | 313 | 1327 | ND | 98.9 |
| Formula (III) | | | | | |
| Cmpd | ND | 118 | 242 | ND | 99.0 |
| Formula (III) | | | | | |
| RH | | | | | |

1.1 g of the compound of formula (III) was weighed into a round bottom flask equipped with a vacuum gauge, vacuum adapter, and a nitrogen flow that passes through a flask filled with water. The solids were evacuated to −20″ Hg while passing a stream of wet nitrogen through the flask at room temperature overnight before being sampled for GC and HPLC. See Table 3.2 for results.

TABLE 3.2
Experiment Results

| | | | | Dimethyl- | HPLC |
|---|---|---|---|---|---|
| | Acetonitrile | Methanol | Acetone | formamide | Purity |
| Sample | (ppm) | (ppm) | (ppm) | (ppm) | (% AUC) |
| Cmpd | 123 | 313 | 1327 | ND | 98.9 |
| Formula (III) | | | | | |
| Cmpd | ND | 94 | 46 | ND | 98.9 |
| Formula (III) | | | | | |
| RH | | | | | |

Inside a nitrogen purged glove bag, 100.8 g of the compound of formula (III) was weighed into a drying tray. The drying tray was placed into the vacuum oven and evacuated. The vacuum was adjusted to ˜−25″ Hg using a stream of nitrogen bubbling through a flask filled with water. The solids were evacuated for 63 hours and 45 minutes before releasing vacuum with a stream of wet nitrogen. The solids were left under a sweep of wet nitrogen for 78 hours 15 minutes prior to packaging. Hydrating afforded 118.0 g of a dark purple solid.

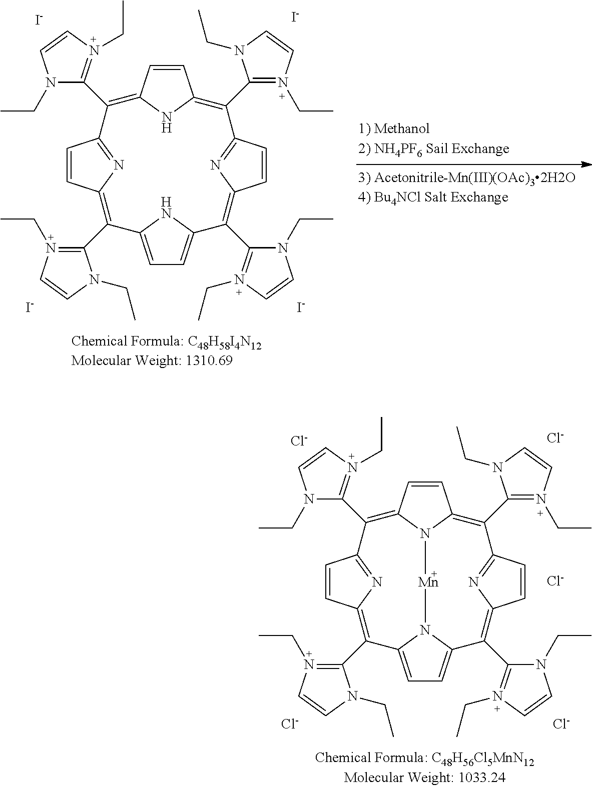

Synthesis of Hexafluorophosphate Salt Intermediate. To a 100 L jacketed reactor equipped with a condenser, nitrogen inlet, thermocouple, and an overhead stirrer was charged the porphyrin SM (1386 g) and methanol (54 kg). The mixture was heated to 59° C. and held for 40 minutes. A solution of ammonium hexafluorophosphate (2.07 kg, purchased from Aldrich) in methanol (17.2 kg) was charged to the mixture through a 0.2 μm filter capsule over 31 minutes. The mixture was allowed to cool to room temperature over 4 hours 2 minutes and filtered (5 μm nylon filter cloth used on an 18″ polyethylene filter). The solids were washed with methanol (2×11.0 kg). Drying under high vacuum at 45±5° C. afforded 1372 g of a dark purple powder. Analysis of the porphyrin hexafluorophosphate salt by HPLC determined the solids to have a purity of 96.3% AUC.

To a 100 L jacketed reactor equipped with a condenser, nitrogen inlet, thermocouple, and an overhead stirrer was charged the porphyrin hexafluorophosphate salt (967 g) and 0.22 μm filtered acetonitrile (25 kg). To the solution was charged manganese (III) acetate dihydrate (377.0 g, purchased from Strem). The mixture was heated to 60° C. and held for 4 hours 17 minutes until analysis by HPLC showed that the porphyrin hexafluorophosphate was not detected. The mixture was cooled to room temperature, drained, and charged back into the 100 L jacketed reactor (cleaned with 0.22 μm filtered water and 0.22 μm filtered acetonitrile) through a 0.22 μm filter capsule. To the filtered solution was charged 0.22 μm filtered purified water (968 g), and the resulting solution was heated to 63° C. A solution of tetrabutylammonium chloride (2.8 kg, purchased from AK Scientific) in acetonitrile (12.7 kg) was charged through a 0.22 μm Teflon filter capsule over 10 minutes. The reaction mixture was cooled to room temperature over 2 hours, held for an additional 2 hours, and filtered (5 μm Nylon filter cloth used in a Pope Scientific agitated Nutsche filter). The solids were washed with 0.22 μm filtered acetone (2×12.7 kg) and dried under vacuum for 16 hours. The solids were hydrated using wet nitrogen with periodic stirring for 30 hours 12 minutes and sampled for residual solvents by GC. The solids were packaged, affording 807 g of an off-brown solid (93% yield).

Differential Scanning Calorimetry (DSC). DSC data were collected using a TA Instruments Q10 DSC. Typically, samples (˜2 mg) were placed in hermetic alodined aluminum sample pans and scanned from 30 to 350° C. at a rate of 10° C./minute under a nitrogen purge of 50 mL/minute.

Thermal Gravimetric Analysis (TGA). TGA data were collected using a TA Instruments TGA Q500. Typically, samples (˜10 mg) were placed in an open, pre-tared aluminum sample pan and scanned from 25 to 350° C. at a rate of 10° C./minute using a nitrogen purge at 60 mL/minute.

X-ray Powder Diffractometer (XRPD). X-ray powder diffraction patterns were obtained using a Bruker D8 Advance equipped with a Cu Kα radiation source (1.54° A), a 9-position sample holder and a LYNXEYE Super Speed Detector. Typically, the duration of each scan was 180 seconds and the 2a range was 4 to 40°. Samples were placed on zero-background, silicon plate holders.

Dynamic Vapor Sorption (DVS). Samples were analyzed using an AQUADYNE™ DVS-2 gravimetric water sorption analyzer. The relative humidity was adjusted between 2-95% and the weight of sample was continuously monitored and recorded.

### Example 4

Solubility Assessment. About 50 mg of solid was slurried in 0.75 mL of various solvents for one day. The slurry was centrifuged and the clear solution was used for gravimetric method. Table 4.1 presents the solubility data measured using this method in various solvents. About 10% error in measurement is expected.

TABLE 4.1
Solubility

| | Solubility | |
|---|---|---|
| Solvent | (mg/mL) 25° C. | Solubility (mg/mL) 45° C. |
| Heptane | 3 | 5 |
| Toluene | 6 | 4 |
| Tert-butyl methyl ether | 3 | 6 |
| Ethyl Acetate | 2 | 4 |
| Methyl ethyl ketone | 3 | 5 |
| Tetrahydrofuran | <1 | 10 |
| Isopropanol | >70 | >70 |
| Acetone | — | 3 |
| Ethanol | >70 | >70 |
| Methanol | >70 | >70 |
| Dimethylformamide | >70 | >70 |
| 1,4 dioxane | — | — |
| Acetonitrile | >70 | >70 |
| Water | >70 | >70 |
| Cyclohexane | <1 | 3 |
| Diethyl ether | 7 | 10 |
| Isopropanol:Water (98:2) | >70 | >70 |
| Acetonitrile:water | >70 | >70 |

— = not soluble

pH Stability and Storage Conditions. These studies were intended to determine the optimal concentration of compounds in Water for Injection (WFI), the optimal pH range, and to identify a candidate formulation for long-term stability studies. In all studies, it was attempted to bring a solution of Formula (VI) to an oxidation/reduction endpoint in order to achieve pH and osmolality stability.

The pH-stability profile was generated at a pH range of 4.1-6.5, where the greater physicochemical stability was observed at the lower pH region. For example, the pH 4 samples demonstrated acceptable pH shift within 0.1 pH units and reasonable drug stability below 60° C. storage after 14 days.

Study 1: pH Titration of 75 mg/mL Formula (VI) in WFI with 1.0N HCl.

A solution of 75 mg/mL compound was prepared by dissolving in WFI and gently mixing for 16-24 hours prior to the titration. The titration of compound with a strong acid provided information for this compound in terms of “apparent” pKa.

A molecular compound of formula (VI) consists of 4 groups of imidazole chloride salts that would readily dissolve in WFI and provide a mildly basic solution. Due to relatively high molecular weight (1033) of Formula (VI), the long mixing process is crucial for the completion of drug dissolution and dissociation in order to achieve the pH equilibrium. In addition, this mixing would allow oxidation of trace Mn (II) compound to the higher oxidation state of Mn (III). The drug solution was titrated with 1.0 N HCl at 30 μL increments.

Study 1 Protocol, pH Titration of 75 mg/mL Formula (VI) in WFI with 1.0N HCl.

A 20 mL solution of 75 mg/mL Formula (VI) in WFI was prepared. About 10 g of WFI was placed in the container including stir bar. About 1.50 g of Formula (VI) was then added to the container while mixing. Additional WFI was added to the container to bring the solution weight to 20.60 g (estimated density of 75 mg Formula (VI) in WFI=1.03 g/mL). The solution was them mixed at room temperature for 16-24 hours. At the end of the 16-24 hour hold/mix, the solution was titrated from its starting pH of about 9 down to pH 3 using 1N HCl.

Study 2: 75 mg/mL Formula (VI) in WFI at pH 7.0 (14 days at 60° C.).

Formula (VI) was dissolved in WFI to a concentration of 75 mg/mL and gently mixed for 16-24 hours prior to adjusting the target pH 7.0 using either HCl or NaOH solution. The samples were filled in glass vials and capped with an air headspace. All samples were tested and evaluated for physicochemical stability under the storage conditions at 2-8 and 60° C. after 0, 3, 7 and 14 days. The lower the pH, the greater the drug stability.

Study 2 Protocol, 75 mg/mL Formula (VI) in WFI at pH 7.0 (14 days at 60° C.).

A 20 mL solution of 75 mg/mL Formula (VI) in WFI was prepared. About 10 g of WFI was placed in the container including a stir bar. About 1.50 g of Formula (VI) was added to the container while mixing. Additional WFI was then added to the container to bring the solution weight to 20.60 g (estimated density of 75 mg Formula (VI) in WFI is 1.03 g/mL).

Test two methods for bringing Formula (VI) to an oxidation/reduction endpoint in order to achieve Solution pH and osmolality stability.

Solution #1: About 10.3 g of the 75 mg/mL Formula (VI) solution was transferred into a different container and mixed at room temperature for 16-24 hours. The pH was measured at approximately hourly intervals and at about 16 hours and 24 hours. At the end of the 16-24 hour hold/mix, the pH of the solution was adjusted to pH 7.0 with either HCl or NaOH and mixed for approximately 15 minutes. The solution was filtered through a PVDF, 0.22 μm filter into a clean container and its pH measured.

Solution #2: The remaining 10.3 g of 75 mg/mL Formula (VI) solution in the original mixing container, was sparged with compressed air while mixing at room temperature. The pH was measured at 30 minutes then hourly thereafter for the 16-24 hour time period. Once the solution pH and osmolality stabilized, the air sparging was stopped. The solution was adjusted to pH 7.0 with either HCl or NaOH and mixed for about 15 minutes. The solution was filtered through a PVDF, 0.22 μm filter into clean container and the pH measured.

Both samples were then stored at 60° C. and samples taken at 0, 3, 7 and 14 days from both containers and to measure pH.

Soln-1A: Mixed solution for 24 hours at room temperature, open to air, before adjusting pH back to 6.8-7.2, then filtered through PVDF filter. Placed samples on accelerated stability at 60° C.

Soln-1B: Control Solution—Mixed solution for 24 hours at room temperature, open to air, before adjusting pH back to 6.8-7.2, then filtered through PVDF filter. Stored samples at 2°-8° C.

Soln-2A: Sparged compounding solution with air during mixing for about 4.5 hours then immediately adjusted pH to 6.8-7.2. Held samples overnight at room temperature in closed screw capped tube. Filtered solution the next day. Placed samples on accelerated stability at 60° C.

Soln-2B: Sparged compounding solution with air during mixing for about 4.5 hours. Held samples overnight at room temperature in closed screw capped tube. The next day, adjusted pH to 6.8-7.2 and filtered solution. Samples placed on accelerated stability at 60° C.

Study 3: Various Strengths of Formula (VI) in WFI at pH 7.0 (14 days at 2-8 and 60° C.).

The higher strengths of Formula (VI) in water were evaluated for physicochemical stability at 65, 75 and 100 mg/mL. Ascorbic acid was also included in this study. In this study, the samples were only prepared using a long mixing process as the pH was found to be more stable (less shift) than that from the air sparging ones. The samples were tested and evaluated for physicochemical stability under 2-8 and 60° C. storage conditions after 0, 3, 7 and 14 days.

pH/Osmolality: Without ascorbic acid, the pH of 65 and 75 mg/mL samples demonstrated similar pH changes as previously seen in the Study-2, where the pH was relatively stable at the refrigerated condition and decreased ˜2 pH units at 60° C. after 14 days. For 100 mg/mL refrigerated sample, the pH increased ˜1 pH unit after 3 day storages prior to stabilizing at 14 days. This indicated the initial mixing time of 100 mg/mL sample was not adequate in order to reach pH equilibrium prior to the pH adjustment. Like the other strengths, the pH of 100 mg/mL sample stored at 60° C. also decreased ˜2 pH units when compared to the control sample after 14 days.

Interestingly, the pH of all refrigerated samples containing ascorbic acid increased ˜1.5 pH units from initial pH after 14 days, whereas that of 60° C. samples decreased ˜2 pH units from initial pH after 3 days and rose back ˜1-1.5 pH units after 14 days.

Study protocol, Various Strengths of Formula (VI) in WFI at pH 7.0 (14 days at 2-8 and 60° C.).

Solution-1: A 20 mL solution of 65 mg/mL Formula (VI) in WFI was prepared. About 10 g of WFI was placed in the container including a stir bar. About 1.30 g of Formula (VI) was added to the container while mixing. Additional WFI was then added to the container to bring the solution weight to 20.52 g (estimated density of 65 mg Formula (VI) in WFI=1.026 g/mL). The pH was measured and the solution was mixed at room temperature for 16-24 hours. At the end of the 16-24 hour hold/mix, the pH of solution was adjusted to pH 7.0 with either HCl or NaOH and mixed for about 15 minutes. The solution was filtered through a PVDF, 0.22 μm filter by discarding about 1 mL prior to placing about 9-10 mL into each of two separate clean containers. The pH of the samples was then measured.

Solution-2: A 20 mL solution of 65 mg/mL Formula (VI)+0.5% Ascorbic Acid in WFI was prepared. About 10 g of WFI was placed in the container including a stir bar. About 1.30 g of Formula (VI) was added to the container while mixing. About 0.1026 g of Ascorbic Acid was added to the container while mixing. Additional WFI was then added to the container to bring the solution weight to 20.52 g (estimated density of 65 mg Formula (VI) in WFI=1.026 g/mL). The pH was measured and the solution was mixed at room temperature for 16-24 hours. At the end of the 16-24 hour hold/mix, the pH of solution was adjusted to pH 7.0 with either HCl or NaOH and mixed for about 15 minutes. The solution was filtered through a PVDF, 0.22 μm filter by discarding 1 mL prior to placing about 9-10 mL into each of two separate clean containers. The pH of the samples was then measured.

Solution-3: A 20 mL solution of 75 mg/mL Formula (VI) in WFI was prepared. About 10 g of WFI was placed in the container including a stir bar. About 1.50 g of Formula (VI) was added to the container while mixing. Additional WFI was then added to the container to bring the solution weight to 20.60 g (estimated density of 75 mg Formula (VI) in WFI is 1.03 g/mL). The pH was measured and the solution was mixed at room temperature for 16-24 hours. At the end of the 16-24 hour hold/mix, the pH of solution was adjusted to pH 7.0 with either HCl or NaOH and mixed for about 15 minutes. The solution was filtered through a PVDF, 0.22 μm filter by discarding about 1 mL prior to placing about 9-10 mL into each of two separate clean containers. The pH of the samples was then measured.

Solution-4: A 20 mL solution of 75 mg/mL Formula (VI)+0.5% Ascorbic Acid in WFI was prepared. About 10 g of WFI was placed in the container including a stir bar. About 1.50 g of Formula (VI) was added to the container while mixing. About 0.103 g of Ascorbic Acid was added to the container while mixing. Additional WFI was then added to the container to bring the solution weight to 20.60 g (estimated density of 75 mg Formula (VI) in WFI is 1.03 g/mL). The pH was measured and the solution was mixed at room temperature for 16-24 hours. At the end of the 16-24 hour hold/mix, the pH of solution was adjusted to pH 7.0 with either HCl or NaOH and mixed for about 15 minutes. The solution was filtered through a PVDF, 0.22 μm filter by discarding about 1 mL prior to placing about 9-10 mL into each of two separate clean containers. The pH of the samples was then measured.

Solution-5: A 20 mL solution of 100 mg/mL Formula (VI) in WFI was prepared. About 10 g of WFI was placed in the container including a stir bar. About 2.00 g of Formula (VI) was added to the container while mixing. Additional WFI was then added to the container to bring the solution weight to 20.80 g (estimated density of 100 mg Formula (VI) in WFI is 1.04 g/mL). The pH was measured and the solution was mixed at room temperature for 16-24 hours. At the end of the 16-24 hour hold/mix, the pH of solution was adjusted to pH 7.0 with either HCl or NaOH and mixed for about 15 minutes. The solution was filtered through a PVDF, 0.22 μm filter by discarding about 1 mL prior to placing about 9-10 mL into each of two separate clean containers. The pH of the samples was then measured.

Solution-6: A 20 mL solution of 100 mg/mL Formula (VI)+0.5% Ascorbic Acid in WFI was prepared. About 10 g of WFI was placed in the container including a stir bar. About 2.00 g of Formula (VI) was added to the container while mixing. About 0.104 g of Ascorbic Acid was added to the container while mixing Additional WFI was then added to the container to bring the solution weight to 20.80 g (estimated density of 100 mg Formula (VI) in WFI is 1.04 g/mL). The pH was measured and the solution was mixed at room temperature for 16-24 hours. At the end of the 16-24 hour hold/mix, the pH of solution was adjusted to pH 7.0 with either HCl or NaOH and mixed for about 15 minutes. The solution was filtered through a PVDF, 0.22 μm filter by discarding about 1 mL prior to placing about 9-10 mL into each of two separate clean containers. The pH of the samples was then measured.

Following preparation of the solutions, one container of each of solutions 1-6 was stored at 2-8° C. The remaining containers for each of solutions 1-6 were stored at 60° C. From each container, a sample was taken at 0, 3, 7 and 14 days and the pH of the solution was measured.

Soln-1: 65 mg/mL Formula (VI) in WFI; Mixed solution for 16-24 hours at room temperature, open to air, before adjusting pH back to 6.8-7.2, then filtered through PVDF filter. One aliquot placed on accelerated stability at 60° C. with Control at 5° C.

Soln-2: 65 mg/mL Formula (VI)+0.5% Ascorbic Acid in WFI; Mixed solution for 16-24 hours at room temperature, open to air, before adjusting pH back to 6.8-7.2, then filtered through PVDF filter. One aliquot placed on accelerated stability at 60° C. with Control at 5° C.

Soln-3: 75 mg/mL Formula (VI) in WFI; Mixed solution for 16-24 hours at room temperature, open to air, before adjusting pH back to 6.8-7.2, then filtered through PVDF filter. One aliquot placed on accelerated stability at 60° C. with Control at 5° C.

Soln-4: 75 mg/mL Formula (VI)+0.5% Ascorbic Acid in WFI; Mixed solution for 16-24 hours at room temperature, open to air, before adjusting pH back to 6.8-7.2, then filtered through PVDF filter. One aliquot placed on accelerated stability at 60° C. with Control at 5° C.

Soln-5: 100 mg/mL Formula (VI) in WFI; Mixed solution for 16-24 hours at room temperature, open to air, before adjusting pH back to 6.8-7.2, then filtered through PVDF filter. One aliquot placed on accelerated stability at 60° C. with Control at 5° C.

Soln-6: 100 mg/mL Formula (VI)+0.5% Ascorbic Acid in WFI; Mixed solution for 16-24 hours at room temperature, open to air, before adjusting pH back to 6.8-7.2, then filtered through PVDF filter. One aliquot placed on accelerated stability at 60° C. with Control at 5° C.

Study 4: 75 mg/mL Formula (VI) in WFI at pH 7.0 under ICH Storage Temperatures.

It was found from previous studies that the isotonic solution of 75 mg/mL Formula (VI) in water at pH 7.0 provided a relatively stable pH under the refrigerated condition. However, the pH of this formulation decreased approximately 1-2 pH units at 60° C. after 14 days.

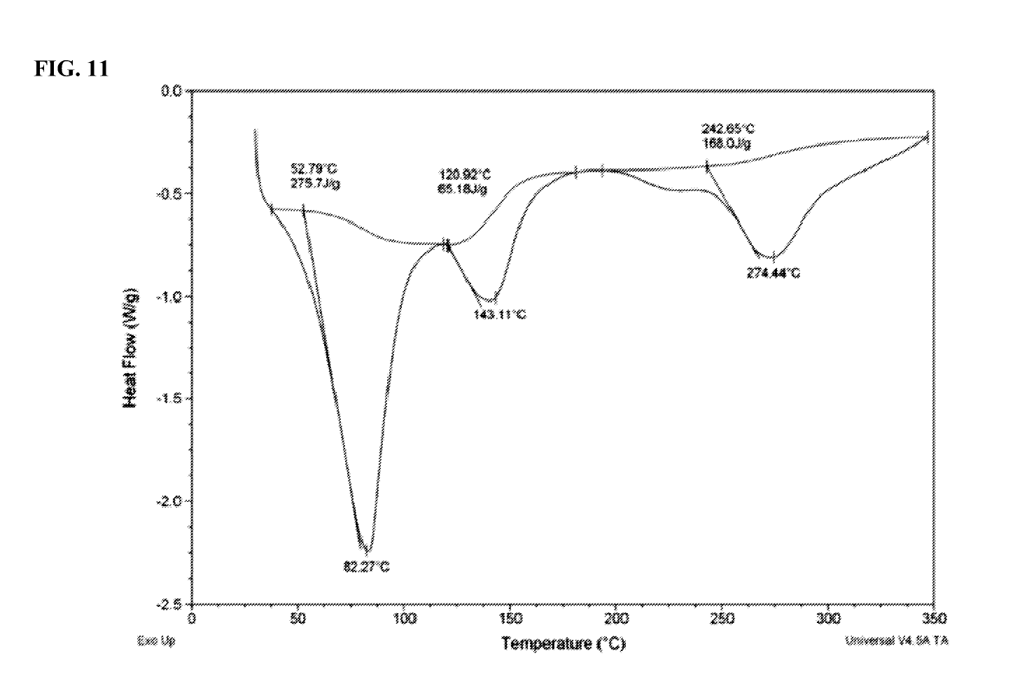

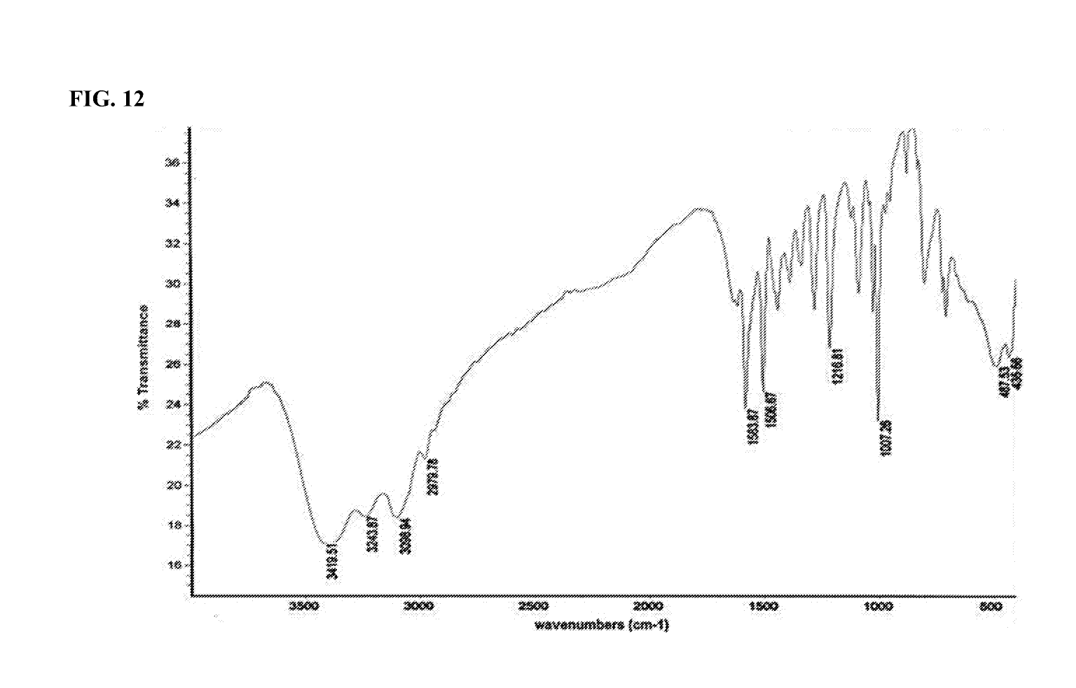

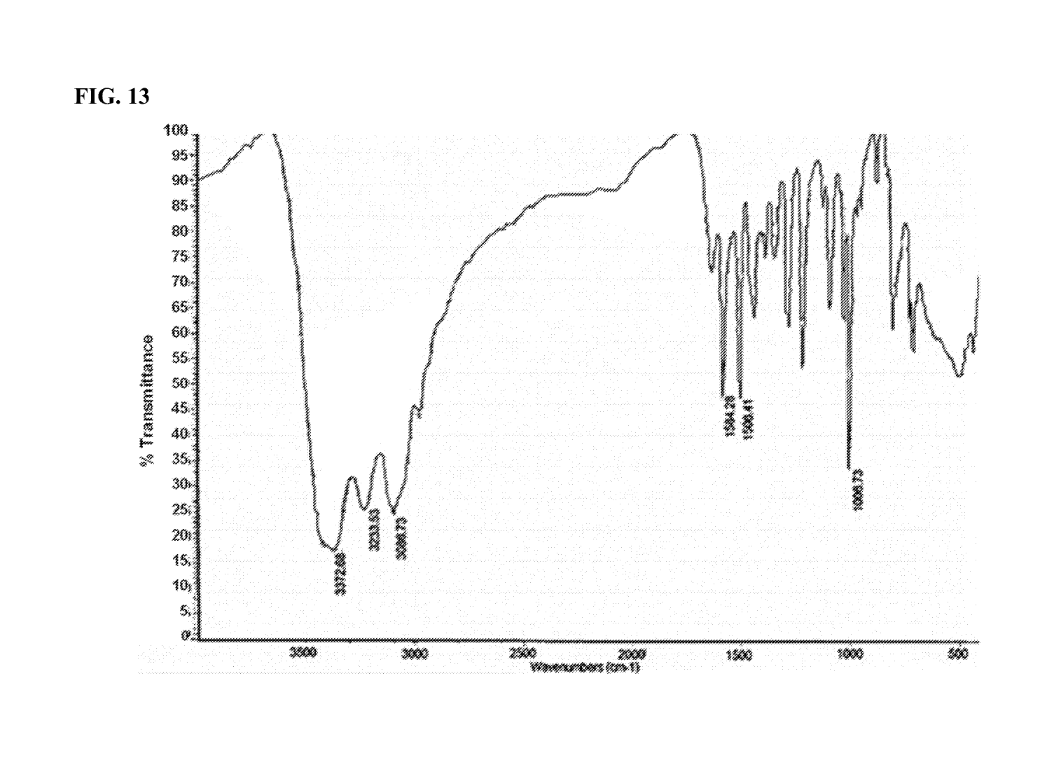

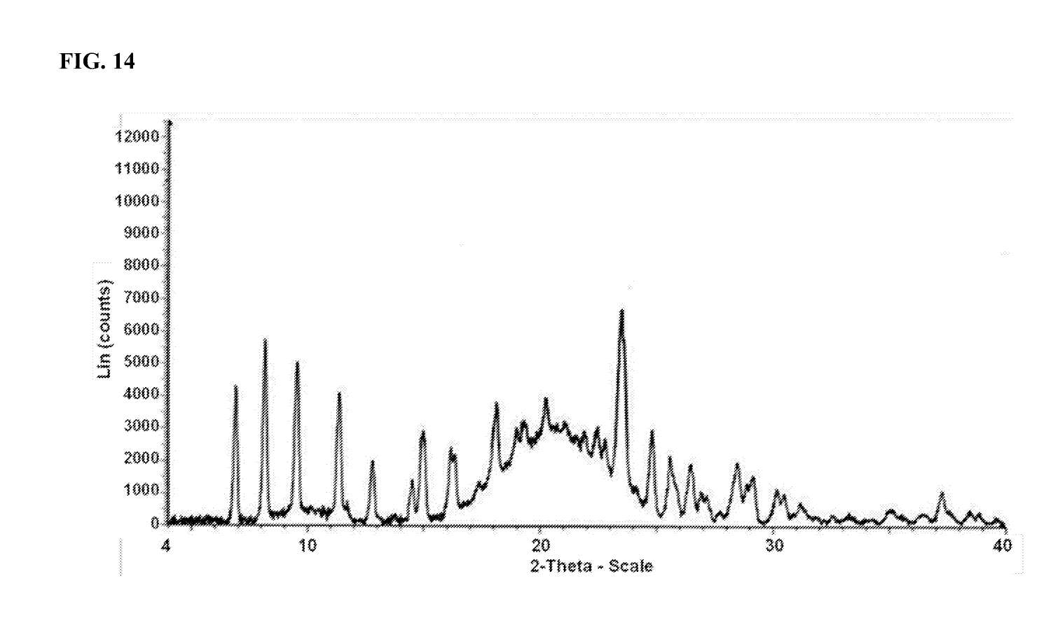

pH/Osmolality: The refrigerated sample provided acceptable stability of pH 7 within 0.1 pH unit after 1 month, while the pH of samples at 25, 30 and 40° C. decreased approximately 0.3, 0.5 and 1.1 pH units, respectively (FIG. 11). All samples provided the isotonic solution (270-276 mOsm/kg) without any significant change of osmolality after 1 month.

Study Protocol, 75 mg/mL Formula (VI) IN WFI at pH 7.0 under ICH Storage Temperatures.

A 20 mL solution of 75 mg/mL Formula (VI) in WFI was prepared. About 10 g of WFI was placed in the container including a stir bar. About 1.50 g of Formula (VI) was added to the container while mixing. Additional WFI was then added to the container to bring the solution weight to 20.60 g (estimated density of 75 mg Formula (VI) in WFI is 1.03 g/mL). The pH was measured and the solution was mixed at room temperature for 16-24 hours. At the end of the 16-24 hour hold/mix, the pH of solution was adjusted to pH 7.0 with either HCl or NaOH and mixed for about 15 minutes. The solution was filtered through a PVDF, 0.22 μm filter by discarding about 1 mL prior to placing about 4-5 mL into each of four separate clean containers (A, B, C, D). The pH of the samples was then measured. Solution A was stored at 2-8° C., solution B at 25° C., solution C at 30° C. and solution D at 40° C. A sample was removed from each container after 3, 7 and 14 days the pH measured.

Study 5: 75 mg/mL Formula (VI) in WFI at Various pH under ICH Storage Temperatures.

It was noticeable from Study 2 that drug stability was dependent on the pH. The lower the pH, the greater the chemical stability. Thus in this study, the chemical stability of 75 mg/mL Formula (VI) in water was evaluated at the pH range at 4-6 under the ICH storage temperatures i.e. 2-8, 25 and 40° C. An accelerated 60° C. storage temperature was also accessed in order to compare and generate a pH-stability profile of drug in water.

The dependence of chemical stability on pH was demonstrated from 60° C. samples, where a decrease of purity assay (˜3%) was found between pH 4.1 and 5.2.

By including the data from previous study of 75 mg/mL Formula (VI) at 60° C. for 14 days, a pH-stability profile was generated between pH 4.1 and 6.5. The increase of pH in such range yielded ˜5% decrease in drug purity assay. All other degradation products increased as a function of pH For instance a degradant at RRT 1.56-1.62 increased ˜8 folds (0.4-3.2%) within the pH profile range.

pH/Osmolality: The stability at pH 4 and 5 were well maintained after 14 days at all storage conditions within 0.1 pH unit variation except slight decrease ˜0.2 pH units of pH 5 at 60° C. The pH shifts were found in both directions at pH 6, where the changes were determined to be 0.7, 0.5, −0.1 and −0.9 pH units after 14 days under the storage conditions at 2-8, 25, 40, and 60° C., respectively.

After 14 days under ICH storage conditions (2-8, 25 and 40° C.), all pH 4, 5 and 6 samples provided isotonic solutions to be 277-280, 273-275 and 269-272 mOsm/kg, respectively. At 60° C. storage, the osmolality of pH 4, 5 and 6 samples were increased to be 292, 302 and 283, respectively.

75 mg/mL Formula (VI) IN WFI at Various pH under ICH Storage Temperatures.

Solution 1: pH 4.0: A 20 mL solution of 75 mg/mL Formula (VI) in WFI was prepared. About 10 g of WFI was placed in the container including a stir bar. About 1.50 g of Formula (VI) was added to the container while mixing. Additional WFI was then added to the container to bring the solution weight to 20.60 g (estimated density of 75 mg Formula (VI) in WFI is 1.03 g/mL). The pH was measured and the solution was mixed at room temperature for 16-24 hours. At the end of the 16-24 hour hold/mix, the pH of solution was adjusted to pH 4.0 with either HCl or NaOH and mixed for about 15 minutes. The solution was filtered through a PVDF, 0.22 μm filter by discarding about 1 mL prior to placing about 4-5 mL into each of four separate clean containers (A, B, C, D). The pH of the samples was then measured. Solution A was stored at 2-8° C., solution B at 25° C., solution C at 30° C. and solution D at 40° C. A sample was removed from each container after 3, 7 and 14 days the pH measured.

Solution 2: pH 5.0 A 20 mL solution of 75 mg/mL Formula (VI) in WFI was prepared. About 10 g of WFI was placed in the container including a stir bar. About 1.50 g of Formula (VI) was added to the container while mixing. Additional WFI was then added to the container to bring the solution weight to 20.60 g (estimated density of 75 mg Formula (VI) in WFI is 1.03 g/mL). The pH was measured and the solution was mixed at room temperature for 16-24 hours. At the end of the 16-24 hour hold/mix, the pH of solution was adjusted to pH 5.0 with either HCl or NaOH and mixed for about 15 minutes. The solution was filtered through a PVDF, 0.22 μm filter by discarding about 1 mL prior to placing about 4-5 mL into each of four separate clean containers (A, B, C, D). The pH of the samples was then measured. Solution A was stored at 2-8° C., solution B at 25° C., solution C at 30° C. and solution D at 40° C. A sample was removed from each container after 3, 7 and 14 days the pH measured.

Solution 3: pH 6.0 A 20 mL solution of 75 mg/mL Formula (VI) in WFI was prepared. About 10 g of WFI was placed in the container including a stir bar. About 1.50 g of Formula (VI) was added to the container while mixing. Additional WFI was then added to the container to bring the solution weight to 20.60 g (estimated density of 75 mg Formula (VI) in WFI is 1.03 g/mL). The pH was measured and the solution was mixed at room temperature for 16-24 hours. At the end of the 16-24 hour hold/mix, the pH of solution was adjusted to pH 6.0 with either HCl or NaOH and mixed for about 15 minutes. The solution was filtered through a PVDF, 0.22 μm filter by discarding about 1 mL prior to placing about 4-5 mL into each of four separate clean containers (A, B, C, D). The pH of the samples was then measured. Solution A was stored at 2-8° C., solution B at 25° C., solution C at 30° C. and solution D at 40° C. A sample was removed from each container after 3, 7 and 14 days the pH measured.

Effect of Formula (VI) on Solution pH: A titration of Formula (VI) compound in water with hydrochloric acid demonstrated a typical titration profile of weak basic drug and strong acid with an “apparent” pKa of 9.02. Due to a big molecular structure (MW=1033), the sample preparation required an unusually long mixing process for 16-24 hours in order to complete the dissociation of drug and the oxidation of trace Mn(III) compound into a higher oxidation state of Mn (III). This mixing process was crucial to achieve the final pH equilibrium.

Without being bound to any particular theory, the chemistry occurring when the pH of the 75 mg/mL solution rises from 4 to 9 over the 16-24 hour period may result from the presence of different oxidation states of Mn(II) and (III). While relatively stable in air in solid state, the Mn(II) form rapidly oxidizes by air in aqueous solution, containing 0.1% TFA with half-life approximately equal 5-7 minutes.

4MnP²⁺+O₂+4H⁺=4MnP³⁺+2H₂O.

One proton is consumed per one molecule of Mn (II) porphyrin complex in the oxidation process. In the absence of acid (for example in WFI water) the oxidation process is expected to be slower and generates hydroxyl-ions:

4MnP²⁺+O₂+2H₂O=4MnP³⁺+4OH⁻

### Example 5

Evaporative Crystallization. Evaporative crystallization data is presented in Table 5.1. Only the solvents that the API had enough solubility resulted in some solid. The rest either did not produce solid or resulted in a gel-like material.

TABLE 5.1
Evaporative crystallization solvent solubility

| Solvent | Form |
|---|---|
| n-Heptane | — |
| Toluene | — |
| tBME | — |
| Ethyl acetate | — |
| MEK | — |
| THF | — |
| 2-propanol | I |
| Acetone | — |
| Ethanol | I |
| | I |
| | — |
| Acetonitrile | — |
| Water | I |
| Cyclohexane | — |
| DEE | — |
| IPA:water (98:2) | I |
| Acetonitrile:water (98:2) | — |

— = no crystal observed

Antisolvent Crystallization. Using the solubility data, a series of antisolvent crystallization experiments were conducted and reported in Table 5.2. As shown in Table 5.2, five solvents and three antisolvents were used in these studies. The solid was dissolved in the solvent at room temperature. Since the solution was fairly dark and dissolution could not be confirmed visually, the vials were centrifuged and the supernatant was used for crystallization. The same solvent systems were used for reverse addition where the dissolved and clarified solution was added to antisolvent at once. The results are reported in Table 5.3. For reverse addition, the majority of cases resulted in oiling out, indicating that crystallization of the API needs to be relatively slow to allow for proper cluster formation and crystallization.

TABLE 5.2
Antisolvent recrystallization

| | | | | | Anti- | | |
|---|---|---|---|---|---|---|---|
| | | | | Solvent, | solvent, | XRPD, | |
| | Solvent | Antisolvent | API, mg | μL | μL | wet | XRPD, dry |
| 1 | IPA | Heptane | 35 | 280 | 280 | I | I |
| 2 | EtOH | Heptane | 40 | 320 | 640 | — | — |
| 3 | MeOH | Heptane | 33 | 198 | 198 | — | — |
| 4 | IPA:water (98:2) | Heptane | 41 | 568 | 620 | I | I |
| 5 | ACN:water (98:2) | Heptane | 33 | 726 | 363 | I | I |
| 6 | IPA | tBME | 29 | 232 | 232 | I | I |
| 7 | EtOH | tBME | 35 | 280 | 560 | — | — |
| 8 | MeOH | tBME | 30 | 180 | 540 | — | — |
| 9 | IPA:water (98:2) | tBME | 43 | 602 | 500 | V | V (extra peaks) |
| 10 | ACN:water (98:2) | tBME | 33 | 726 | 500 | — | — |
| 11 | EtOH | Ethyl acetate | 40 | 320 | 640 | — | — |
| 12 | IPA | Ethyl acetate | 41 | 328 | 328 | I | I |
| 13 | MeOH | Ethyl acetate | 36 | 216 | 432 | — | — |
| 14 | IPA:water (98:2) | Ethyl acetate | 40 | 560 | 1000 | — | — |
| 15 | ACN:water (98:2) | Ethyl acetate | 31 | 682 | 500 | V | V (extra peaks) |

— = no crystal observed

TABLE 5.3
Antisolvent crystallization (reverse addition)

| | | | | | Anti- | | |
|---|---|---|---|---|---|---|---|
| | | | | Solvent, | solvent, | | |
| | Solvent | Antisolvent | API, mg | μL | μL | XRPD, wet | Observation |
| 1 | IPA | Heptane | 33 | 363 | 500 | I | — |
| 2 | EtOH | Heptane | 40 | 320 | 500 | — | Oiled |
| 3 | MeOH | Heptane | 33 | 198 | 500 | — | Oiled |
| 4 | IPA:water (98:2) | Heptane | 32 | 416 | 500 | — | Very little solid |
| 5 | ACN:water (98:2) | Heptane | 43 | 645 | 500 | — | Oiled |
| 6 | IPA | tBME | 38 | 418 | 500 | I | — |
| 7 | EtOH | tBME | 31 | 248 | 500 | I | — |
| 8 | MeOH | tBME | 38 | 228 | 500 | — | Oiled |
| 9 | IPA:water (98:2) | tBME | 32 | 416 | 500 | — | Very little solid |
| 10 | ACN:water (98:2) | tBME | 40 | 600 | 500 | — | Oiled |
| 11 | EtOH | Ethyl acetate | 40 | 440 | 500 | I + VII | — |
| 12 | IPA | Ethyl acetate | 39 | 312 | 500 | I | — |
| 13 | MeOH | Ethyl acetate | 33 | 198 | 500 | — | Oiled |
| 14 | IPA:water (98:2) | Ethyl acetate | 43 | 559 | 500 | — | Oiled |
| 15 | ACN:water (98:2) | Ethyl acetate | 34 | 510 | 500 | — | Oiled |

— = no crystal observed

Reactive Crystallization. A series of reactive crystallization experiments was performed using the last step conditions. In these experiments the penultimate step was used to prepare the API in acetonitrile. Four stock solutions were prepared according to the last step procedure. The hexafluorophosphate salt was dissolved in X volume acetonitrile at 65° C. X was either 33 volumes or 16 volumes as shown in Table 5.4. Solid manganese (III) acetate dihydrate (3 eq.) was added to the solution and stirred for 2 hrs. at 65° C. The resulting solutions were then filtered using syringe filter. To stock solutions number 2 and 4, water was spiked to achieve 0.5 vol % water content. Furthermore, these solutions were dispensed into 16 vials. Separately, a solution of tetrabutylammonium chloride (tBA-Cl, 15 equivalents) in acetonitrile (10 vol) was prepared and filtered. The tBA-Cl was added to the reaction mixture at 65° C. under two regimes of fast (instant addition) and slow addition which was over 30 minutes. Cooling rate to room temperature was also evaluated. The solids were filtered and washed with acetonitrile while exposed to ambient. The lab relative humidity was in the range of 50-65%. The solid was then transferred onto XRPD plates and analyzed while exposed to ambient. The experiments information and the resulting XRPD are presented in Table 5.4. In all cases, Form I was observed.

TABLE 5.4
Reactive crystallization

| | | | Water vol in | tBA-Cl | | Stock | | | |
|---|---|---|---|---|---|---|---|---|---|
| | | Initial | initial | addition | Cooling | solution, | tBA | Stock | |
| 2031- | Solvent | volume, X | solvent % | rate | rate | mL | soln, mL | soln # | XRPD, wet |
| 13-1 | Acetonitrile | 33 | 0 | 30 mins | 1 hr | 2 | 0.75 | 1 | I |
| 13-2 | Acetonitrile | 33 | 0 | 30 mins | Rapid | 2 | 0.75 | 1 | I |
| 13-3 | Acetonitrile | 33 | 0 | Rapidly | 1 hr | 2 | 0.75 | 1 | I |
| 13-4 | Acetonitrile | 33 | 0 | Rapidly | Rapid | 2 | 0.75 | 1 | I |
| 13-5 | Acetonitrile | 33 | 0.50% | 30 mins | 1 hr | 2 | 0.75 | 2 | I |
| 13-6 | Acetonitrile | 33 | 0.50% | 30 mins | Rapid | 2 | 0.75 | 2 | I |
| 13-7 | Acetonitrile | 33 | 0.50% | Rapidly | 1 hr | 2 | 0.75 | 2 | I |
| 13-8 | Acetonitrile | 33 | 0.50% | Rapidly | Rapid | 2 | 0.75 | 2 | I |
| 13-9 | Acetonitrile | 16 | 0 | 30 mins | 1 hr | 2 | 1.48 | 3 | I |
| 13-10 | Acetonitrile | 16 | 0 | 30 mins | Rapid | 2 | 1.48 | 3 | I |
| 13-11 | Acetonitrile | 16 | 0 | Rapidly | 1 hr | 2 | 1.48 | 3 | I |
| 13-12 | Acetonitrile | 16 | 0 | Rapidly | Rapid | 2 | 1.48 | 3 | I |
| 13-13 | Acetonitrile | 16 | 0.50% | 30 mins | 1 hr | 2 | 1.48 | 4 | I |
| 13-14 | Acetonitrile | 16 | 0.50% | 30 mins | Rapid | 2 | 1.48 | 4 | I |
| 13-15 | Acetonitrile | 16 | 0.50% | Rapidly | 1 hr | 2 | 1.48 | 4 | I |
| 13-16 | Acetonitrile | 16 | 0.50% | Rapidly | Rapid | 2 | 1.48 | 4 | I |

Vapor Diffusion into Solid. Form I was used along with 21 solvent system to evaluate the effect of vapor diffusion on polymorphic behavior. About 2 mL solvent was added to a 20 mL scintillation vial. Furthermore, about 30 mg of solid was added to an open 2 mL HPLC vial and the whole vial was placed inside the bigger vial which contained the solvent. Table 5.5 shows the XRPD after 6 days of exposure. Experiments were designed to provide certain relative humidity as shown in the Table. Ethanol, methanol and plain water turned the solid into a dark brown liquid and resulted in differ XRPD pattern than starting solid. Both methanol and ethanol ended up with a mixture of Form I and Form VII. Form I kept its integrity at relative humidity of up to 85% which was generated using saturated potassium chloride.

TABLE 5.5
vapor diffusion to solid

| | | | XRPD after | |
|---|---|---|---|---|
| 2031- | Solvent | Initial XRPD | 6 days exposure | Observation |
| 11-1 | Acetone | I | A | Solid |
| 11-2 | tBME | I | I | Solid |
| 11-3 | EtOH | I | I + VII | Liquid |
| 11-4 | EtOAc | I | I | Solid |
| 11-5 | DEE | I | I | Solid |
| 11-6 | Acetonitrile | I | I | Solid |
| 11-7 | THF | I | I | Solid |
| 11-8 | DCM | I | I | Solid |
| 11-9 | 1,4 Dioxane | I | I | Solid |
| 11-10 | Heptane | I | I | Solid |
| 11-11 | IPAc | I | I | Solid |
| 11-12 | MEK | I | I | Solid |
| 11-13 | IPA | I | I | Gel-like |
| 11-14 | MeOH | I | I + VII | Liquid |
| 11-15 | ACN:water (98:2) | I | I | Solid |
| 11-16 | Saturated NaOH (8% RH) | I | I | Solid |
| 11-17 | Saturated K2CO3 (43% RH) | I | I | Solid |
| 11-18 | Saturated Potassium Iodide (69% RH) | I | I | Solid |
| 11-19 | Saturated Sodium Chloride (75% RH) | I | I | Solid |
| 11-20 | Saturated Potassium Chloride (85% RH) | I | I | Solid |
| 11-21 | Water (>95% RH) | I | VI | Liquid |

Drying and Thermal Treatment Studies. A sample was produced using 3 eq. of Mn(III) acetate. The slurry was filtered at ambient without any precautions. The relative humidity of the lab was at 54% at the time of filtration. The wet cake was washed with acetonitrile followed by XRPD analysis which conformed to Form I. The wet cake was dried on a XRPD plate with dome in the over at 40° C., under vacuum for overnight. Then, the sample holder was capped while in the oven followed by XRPD analysis. The resulting solid was a Form III. Then, the cap of the domed holder was opened and allowed the dry solid to be exposed to ambient at RH of 54%. In less than half an hour, the solid was fully converted to Form I which is a hydrate.

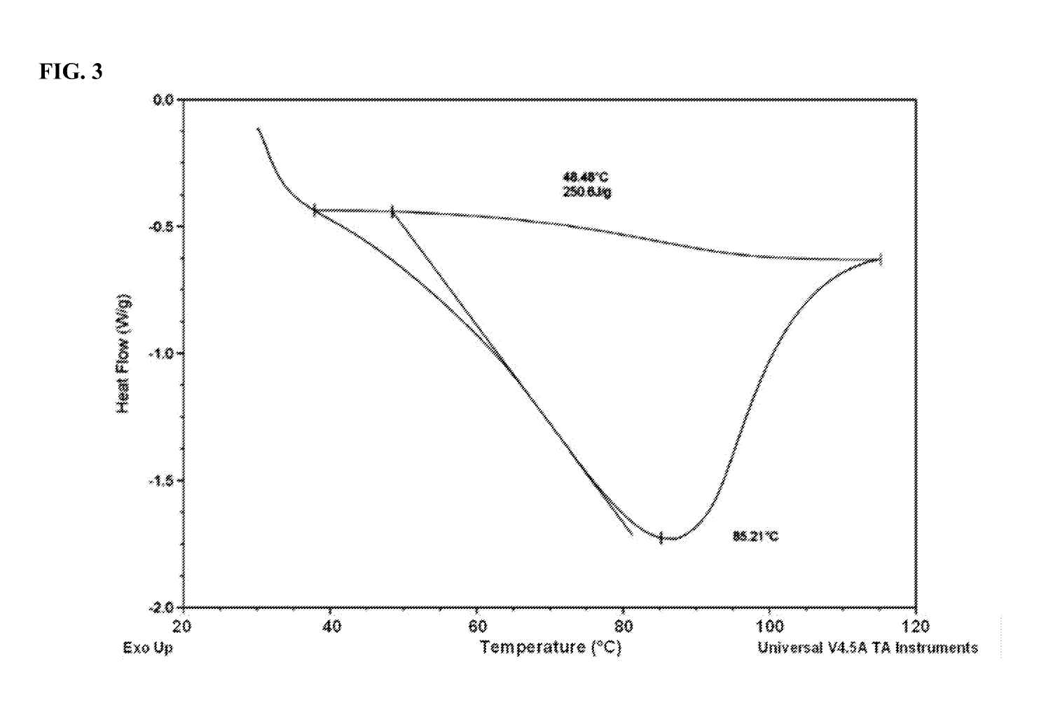

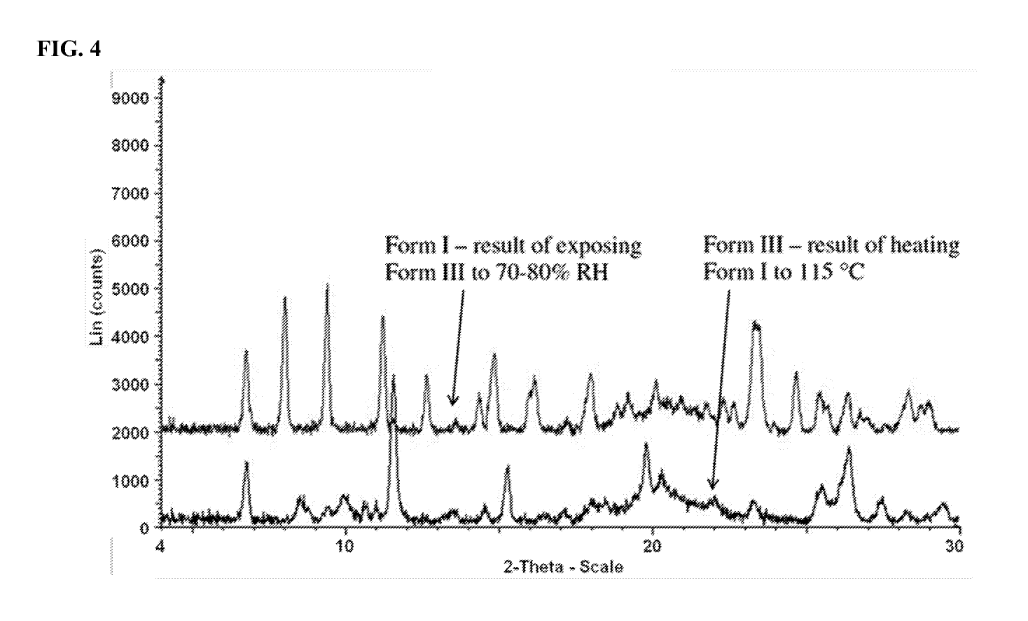

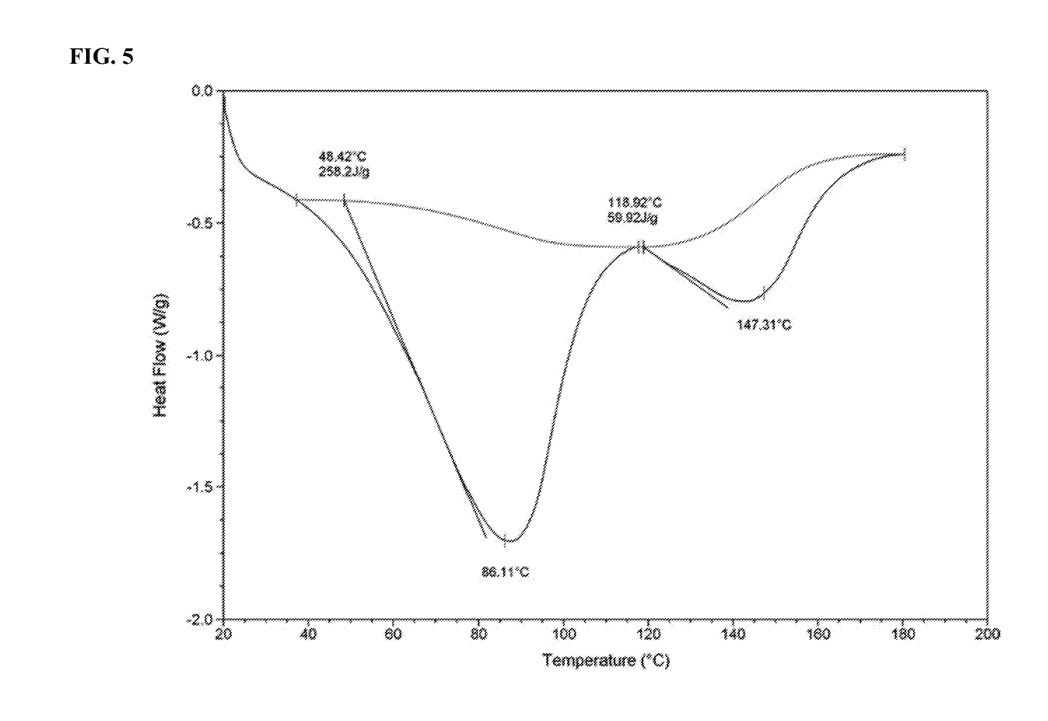

Form I was used to evaluate the effect of thermal treatment. DSC of Form I shows multiple endothermic peaks. To characterize each of these peaks, Form I was heated to endpoint of the peak using DSC. FIG. 3 shows the DSC thermogram of Form I heated to 115° C. which is just after the first peak. The sample was cooled to room temperature under nitrogen then transferred into a XRPD sample holder with dome. The XRPD is shown in FIG. 4 where it reveals that the crystal form after the first endothermic peak is Form III. Furthermore, this solid was exposed to relative humidity of 70-80% for 15 minutes followed by XRPD analysis which showed Form I. Therefore, the form conversion as a result of the first peak was reversible.

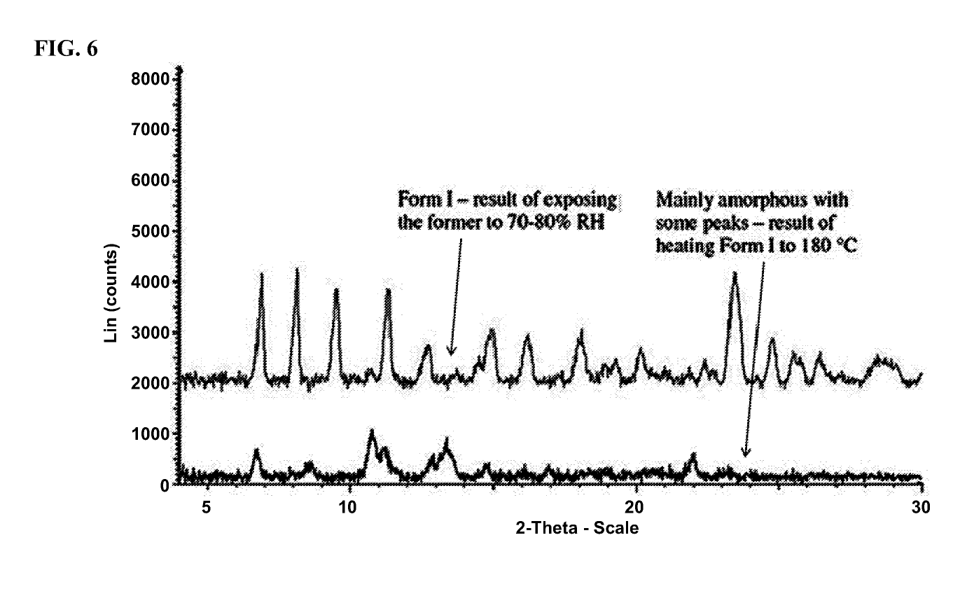

In another experiment, Form I was heated to higher temperature of 180° C. which was the end point of the second endothermic peak. The sample was cooled to room temperature under nitrogen then transferred into a XRPD sample holder with dome. The XRPD is shown in FIG. 6 where it reveals that heating to the end point of the second peak results in mainly amorphous solid with some peaks. After this point, the sample melts/degrades. Furthermore, this solid was exposed to relative humidity of 70-80% for 15 minutes followed by XRPD analysis which showed Form I. Therefore, the form conversion as a result of second peak was also reversible.

Wet and Dry Grinding Studies. Form I was ground using mortar and pestle under dry and wet conditions. See FIG. 7. The solvents in wet grinding were acetonitrile, acetonitrile:water (98:2) and ethyl acetate. This shows that Form I is pretty stable under grinding conditions. It should be noted that the grinding was performed under ambient conditions where relative humidity was around 50-60%.

Competitive Slurry Experiments. Mixture of six crystal forms (I, II, III, V, VI and VII) were slurried in three different solvents (acetonitrile acetonitrile:water (98:2) and ethyl acetate), at 25±2° C. for 5 days followed by filtration under nitrogen inert condition. See FIG. 8. About 20 mg of each polymorph added to the vials. The total weight was about 180 mg in each vial and 0.75 mL solvent was added. After filtration, the cake was washed with the same solvent as the one used in the slurry. The cake was placed on a sample holder and sealed using the X-ray transparent dome and analyzed using XRPD. The cap was then removed and solid was dried at 45° C. and under vacuum for half a day. The dry sample was then sealed under nitrogen inert environment and analyzed by XRPD. The next step was to expose the dry sample to about 50% relative humidity for 30 minutes followed by XRPD analysis. In the case of acetonitrile, the wet cake was a new pattern designated as Form IV. In acetonitrile:water (98:2), the resulting solid was low crystalline Form I. It seemed that 2 vol % water was not enough to result in a crystalline hydrate. In the case of ethyl acetate, the solid was also low crystalline Form I plus a few extra peaks. The water in starting Form I could have been enough to result in a low crystalline Form I with some extra peaks of the starting forms in hydrophobic ethyl acetate. This was not observed in neat acetonitrile due to affinity of this solvent for water. While ethyl acetate does not have the same water affinity as acetonitrile and the water is pushed to the API. Theoretically, the same water quantity in ethyl acetate results in higher water activity than in acetonitrile. Based on these results, and also previous experiments which showed that all the crystal forms convert to Form I upon exposure to moisture, Form I was selected as the most stable crystal form for development.

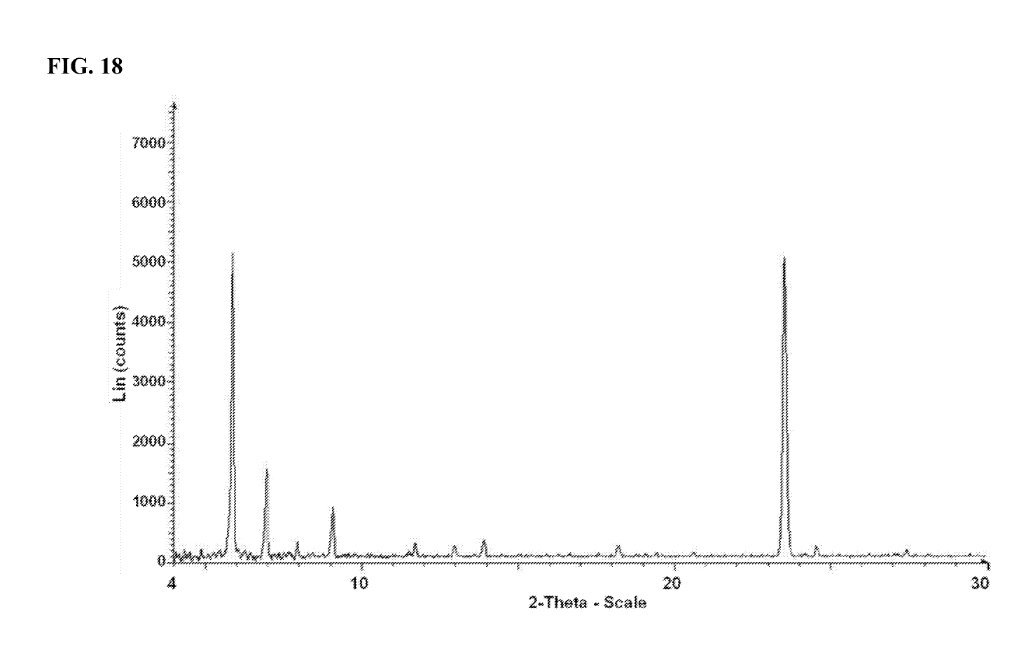

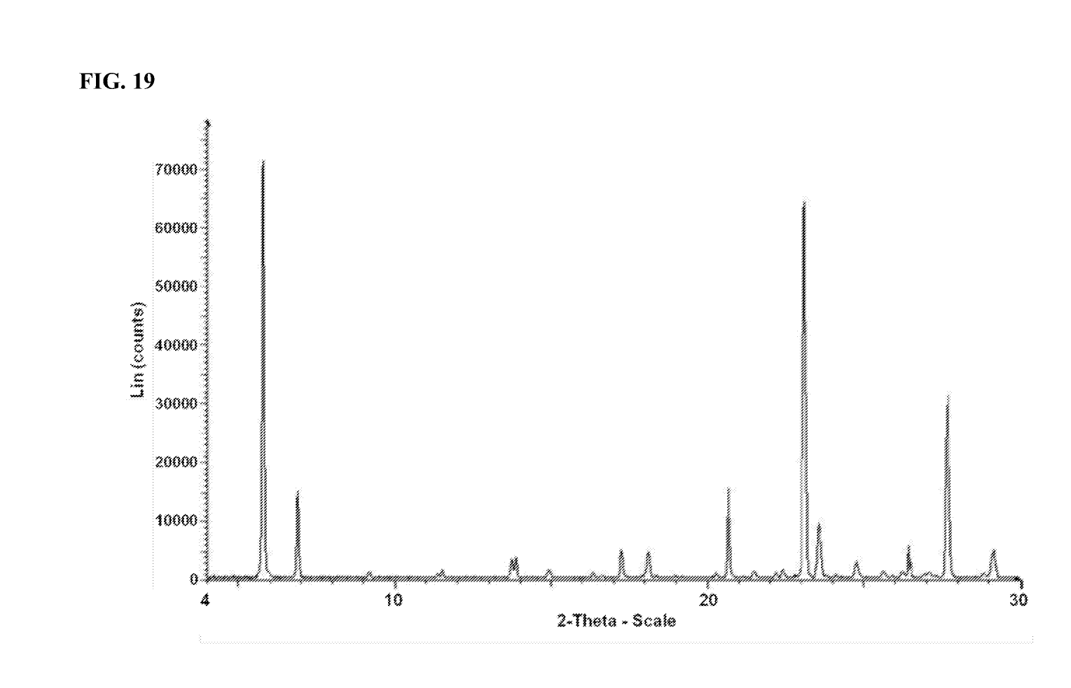

Humidity Stability of Form I. Form I was exposed to a typical relative humidity range that most labs will experience e.g. 15-75% at 25° C. Initially the chamber relative humidity was adjusted at 50%. Then the RH was cycled between 15 to 75% and weight was monitored. FIG. 18 illustrates the changes in weight as a function of relative humidity. If the solid is equilibrated at 50% relative humidity the variation in weight would about ±2 wt % between 15-75% RH. Furthermore, an equilibrium study was performed at various relative humidity environments for extended time. Table 5.6 show the equilibrium % water uptake at various humidity levels.

TABLE 5.6
equilibrium water uptake at various relative humidity conditions

| Relative humidity, % | Weight, mg | % water uptake | Possible Form |
|---|---|---|---|
| 2 | 45.17 | 0.00 | Form III |
| 20 | 51.27 | 13.50 | Form I |
| 40 | 51.95 | 15.01 | Form I |
| 75 | 52.77 | 16.83 | Form I |
| 80 | 53.78 | 19.06 | Form I |

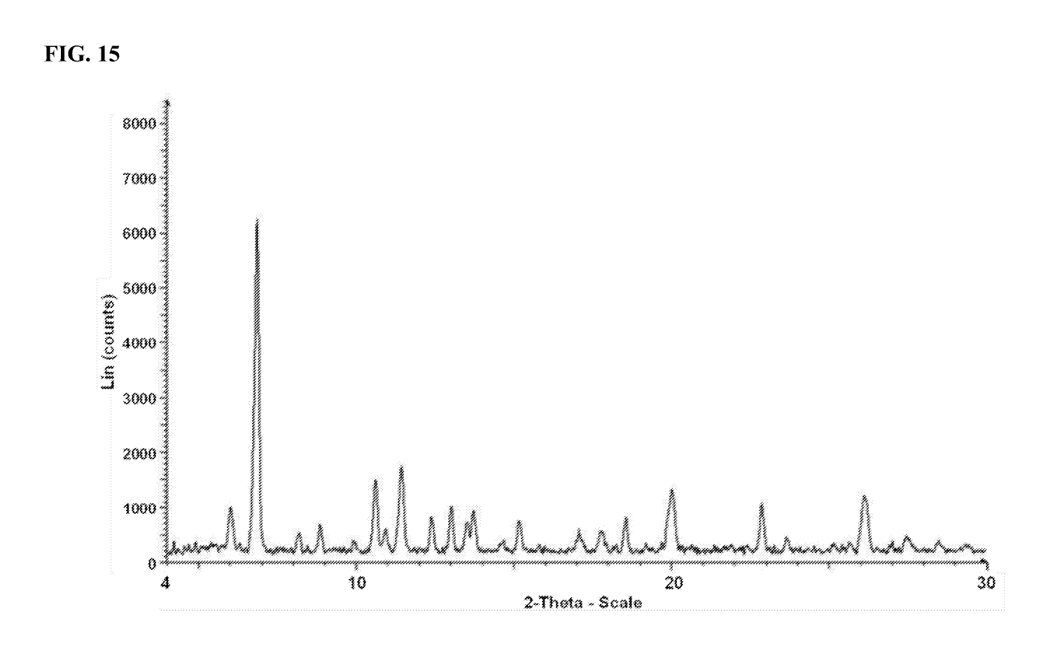

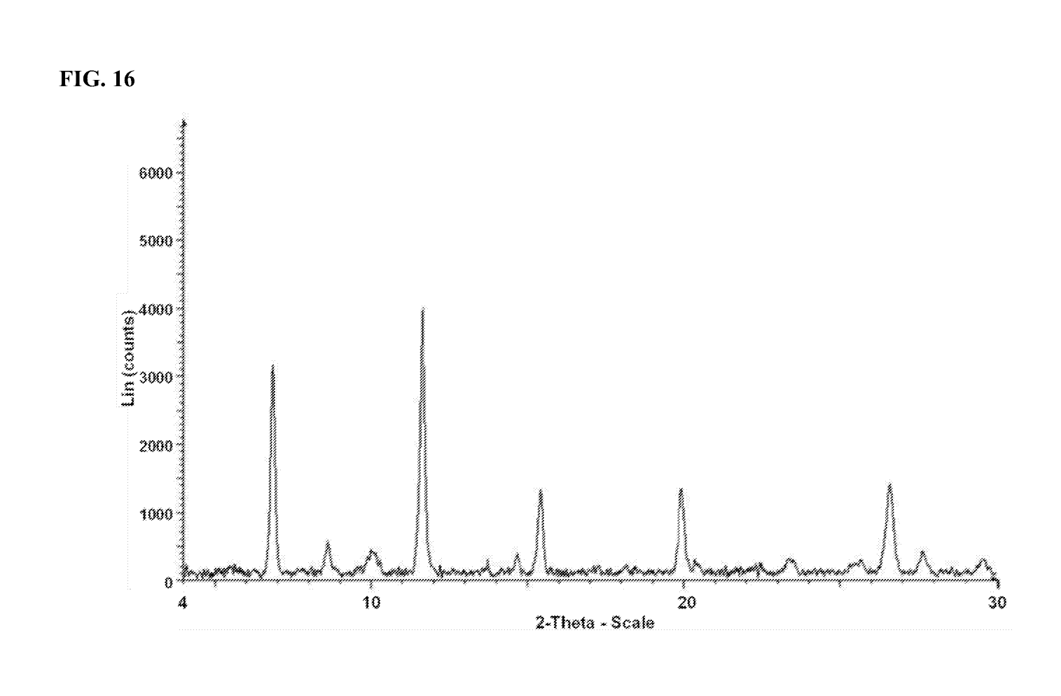

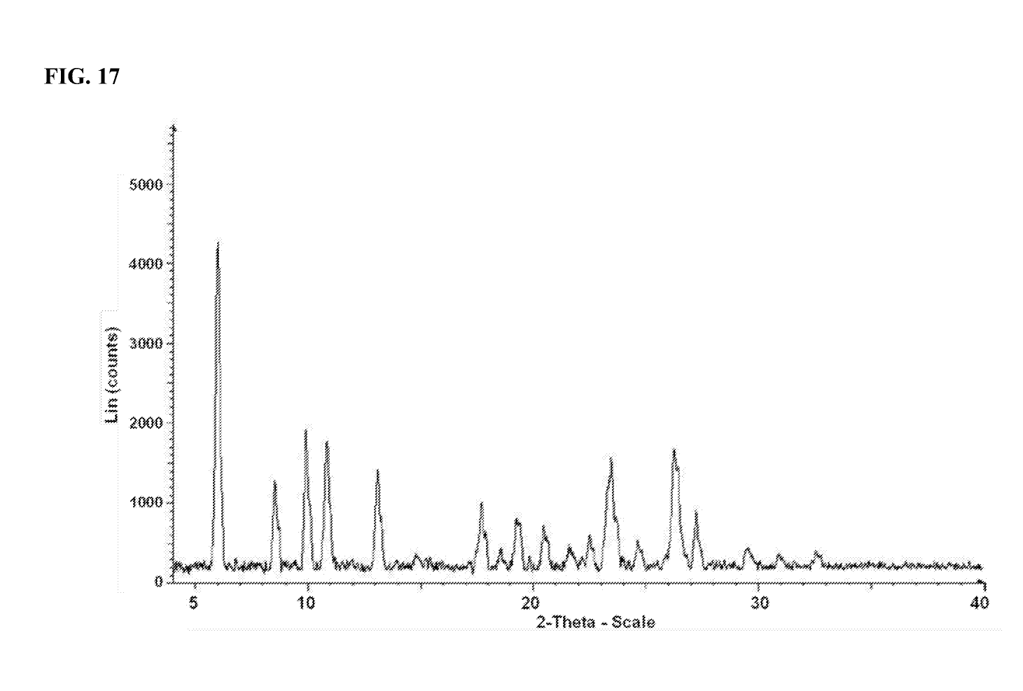

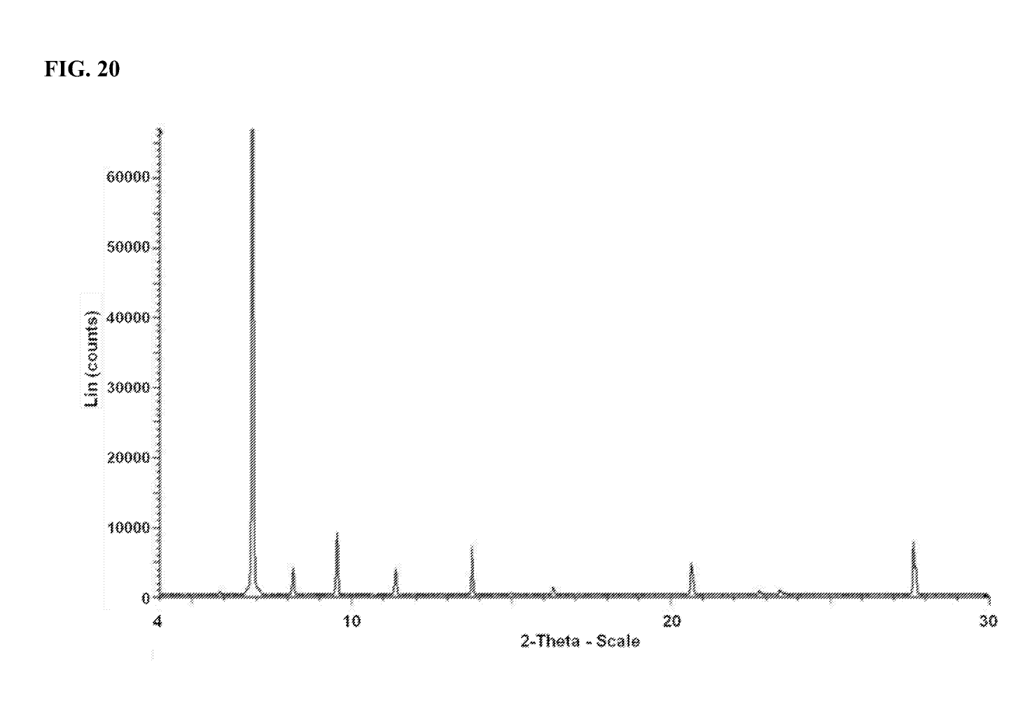

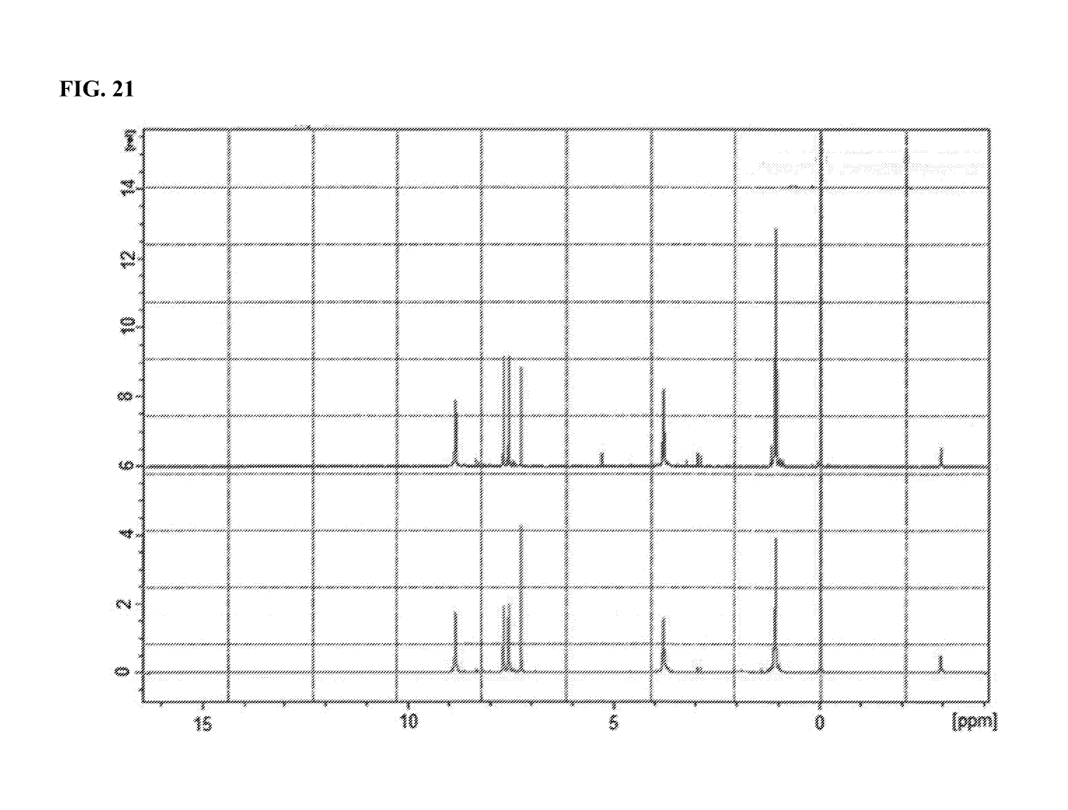

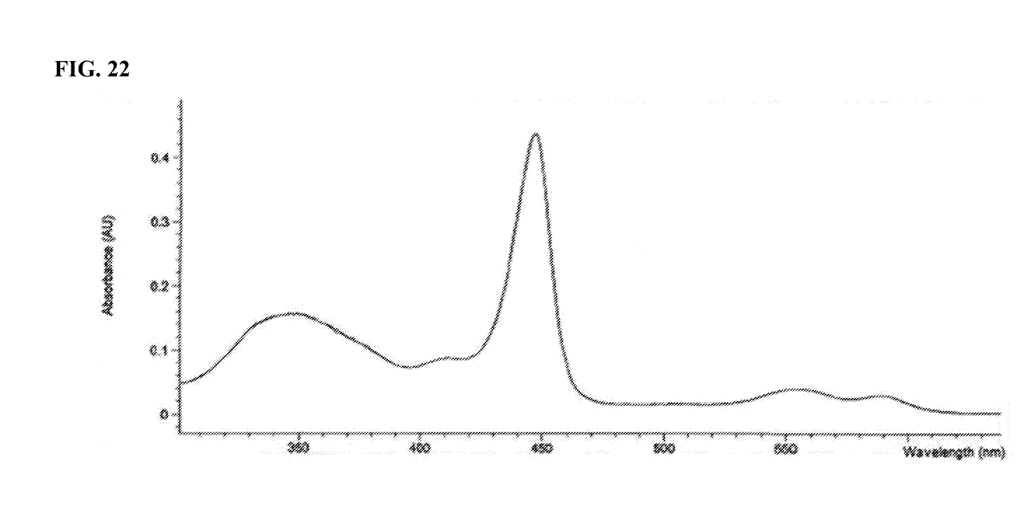

Form II is the wet cake out of reaction mixture unexposed to moisture. Section 3.3.2.3 (reactive crystallization) describes the procedure of making Form II. FIG. 15 illustrates the XRPD of Form II. For XRPD analysis, a silicon plate with dome was used to prevent exposure to ambient. Form III is the result of drying of any of the solid forms. This form is unstable and rapidly converts to Form I upon exposure to moisture. Due to instability, some peaks might be shifted if the same sample is repeated multiple times. FIG. 16 illustrates the XRPD of Form III. For XRPD analysis, a silicon plate with dome was used to prevent exposure to ambient. Form IV is the wet cake from slurrying all the solid forms in acetonitrile for at least 5 days and at room temperature. This form is unstable and upon exposure to moisture, it converts to Form I. FIG. 17 illustrates the XRPD of Form IV. For XRPD analysis, a silicon plate with dome was used to prevent exposure to ambient. Form V is the wet cake from dissolving Form I in IPA:water (98:2) and adding tert-butyl methyl ether as antisolvent. FIG. 18 illustrates the XRPD of Form V. For XRPD analysis, a silicon plate with dome was used to prevent exposure to ambient. Form VI was obtained through expose Form I to moisture of more than 95% for at least 6 days where it converted to a liquid solid. FIG. 29 illustrates the XRPD of Form VI. Form VII was obtained through expose Form I to methanol or ethanol vapor for at least 6 days where it converted to a liquid solid. FIG. 20 illustrates the XRPD of Form VII.

### Example 6

Sample Preparations for Crystallography: Sample of compound containing manganese predominantly in lower oxidation state was prepared according to procedures herein. In brief, in the glove box with complete exclusion of air one gram (0.72 mmol) of the dried hexafluorophosphate salt (lot 1952-20-1) was dissolved in degassed acetonitrile (30 mL). The resulting solution is heated to 65±5° C. and stirred for 30 minutes to ensure dissolution. Solid manganese (II) acetate dihydrate (2.0 g; 8.18 mmol; 11.3 equivalents) was added via a powder funnel. The reaction is stirred at 65±5° C. for 65 hours. The resulting solution was filtered to remove insoluble excess of manganese (IT) acetate. A solution of tetrabutylammonium chloride (2.98 g, 10.7 mmol; 15 equivalents) in acetonitrile (10 mL) is added into the product solution. The reaction mixture was then cooled to 25° C., the solid product collected by vacuum filtration and washed with acetone (2×15 mL). The product was dried under vacuum with exclusion of air at room temperature.

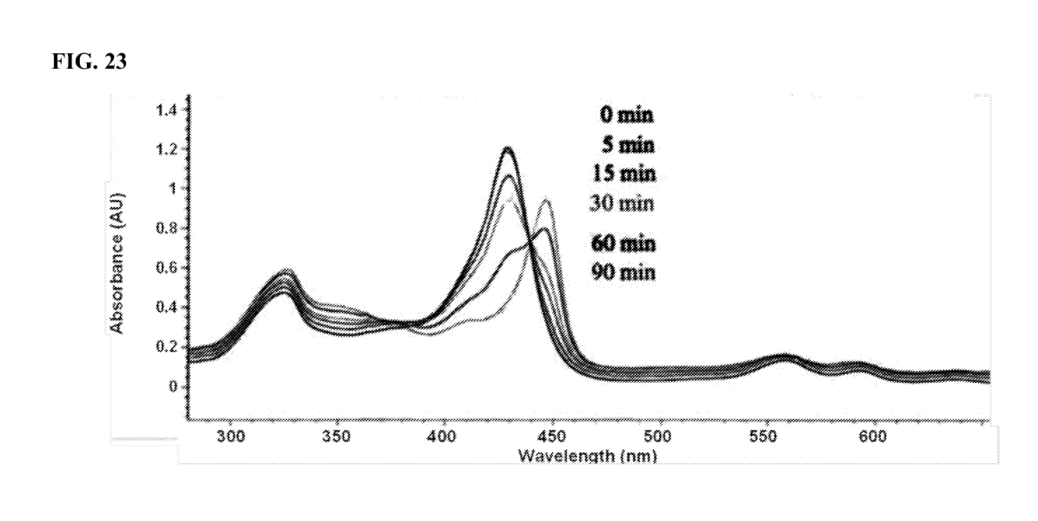

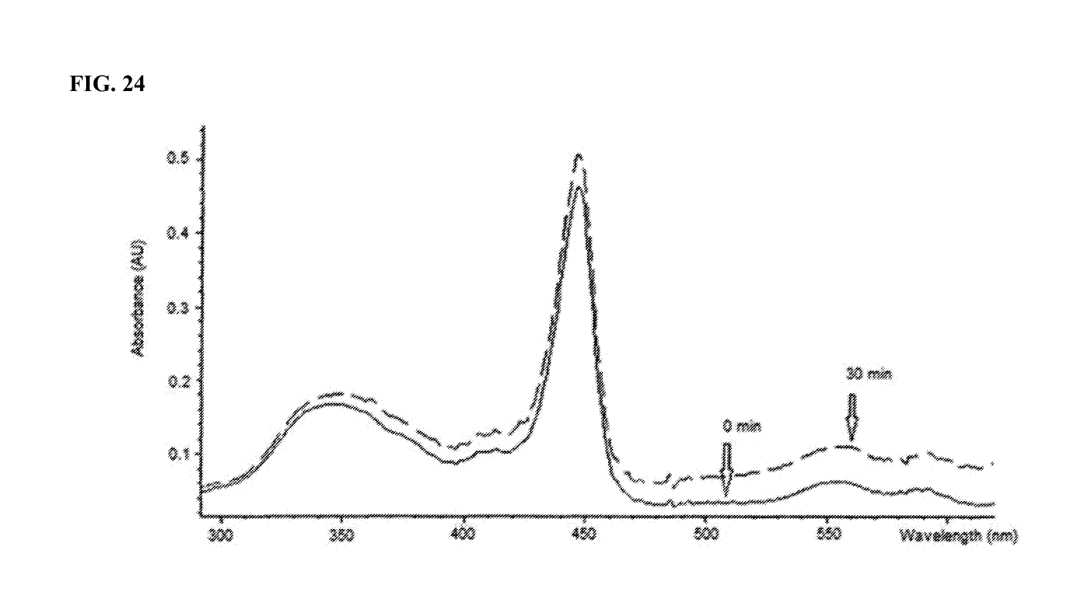

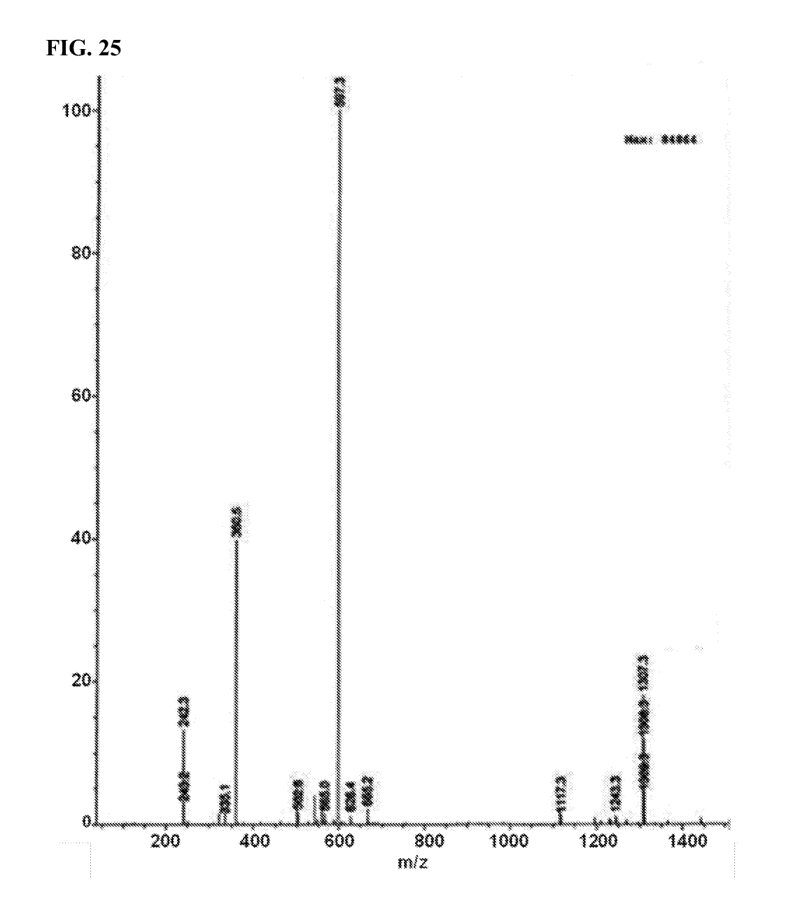

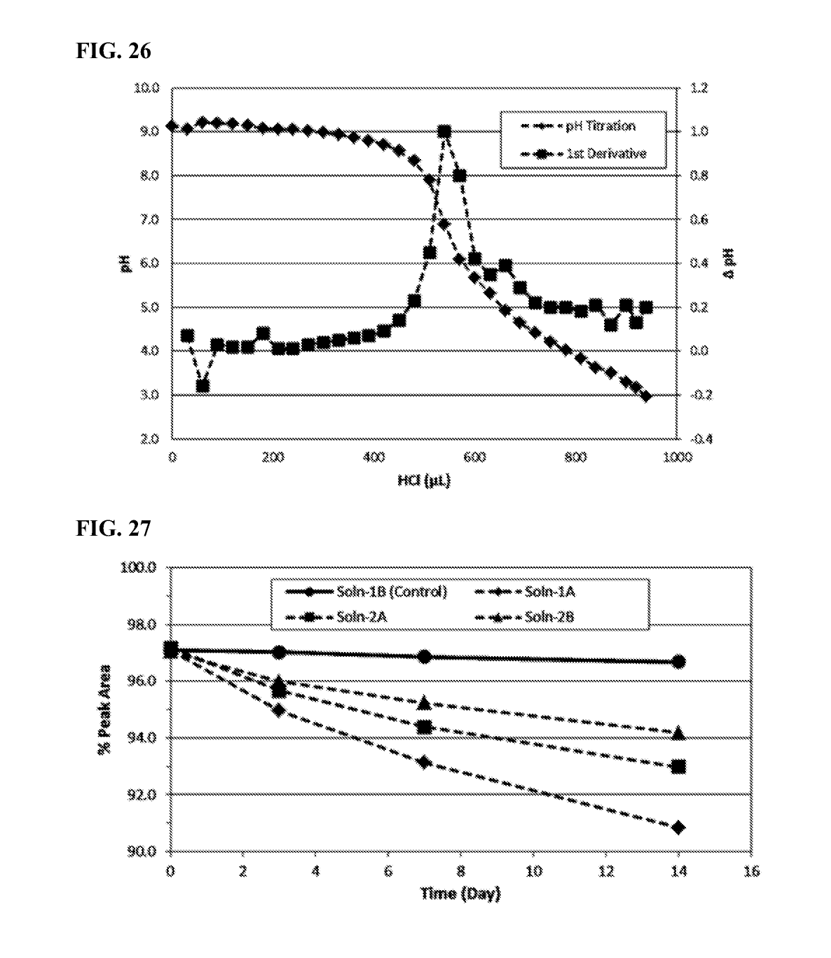

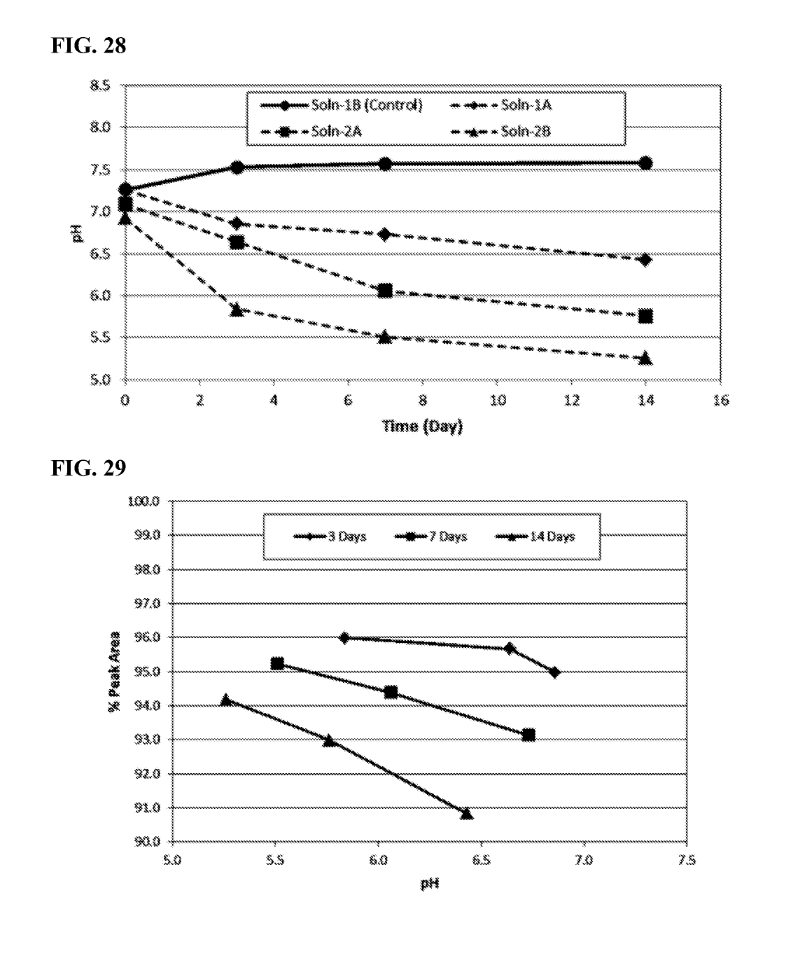

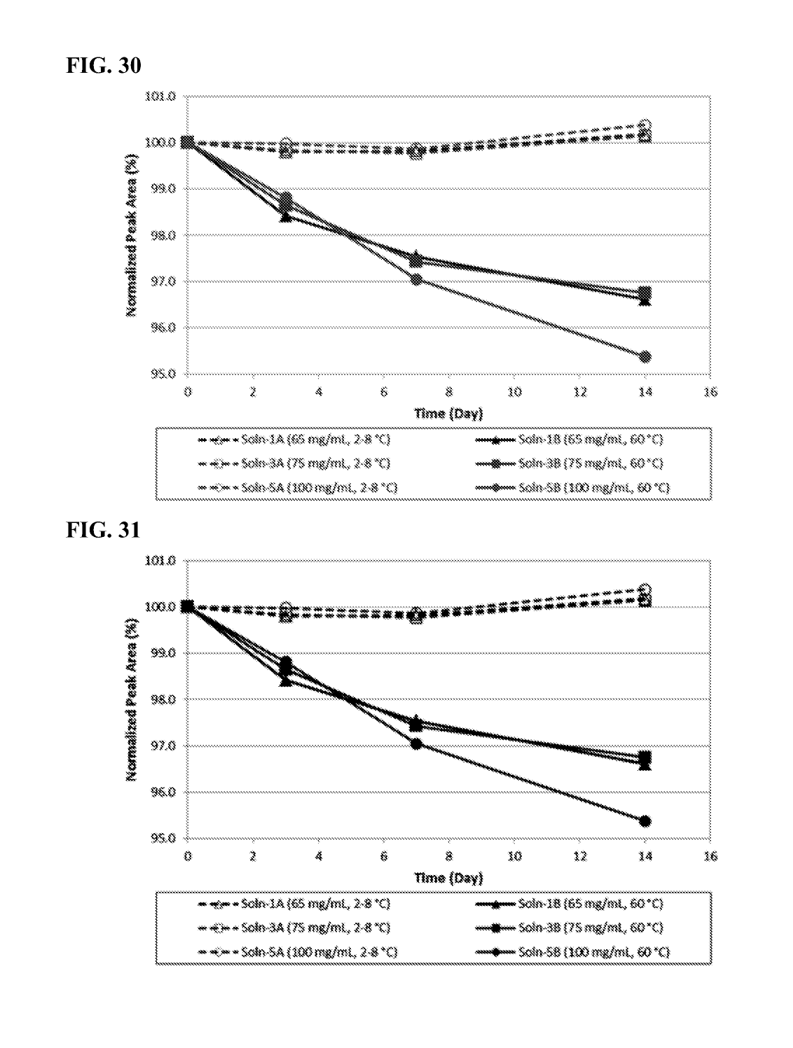

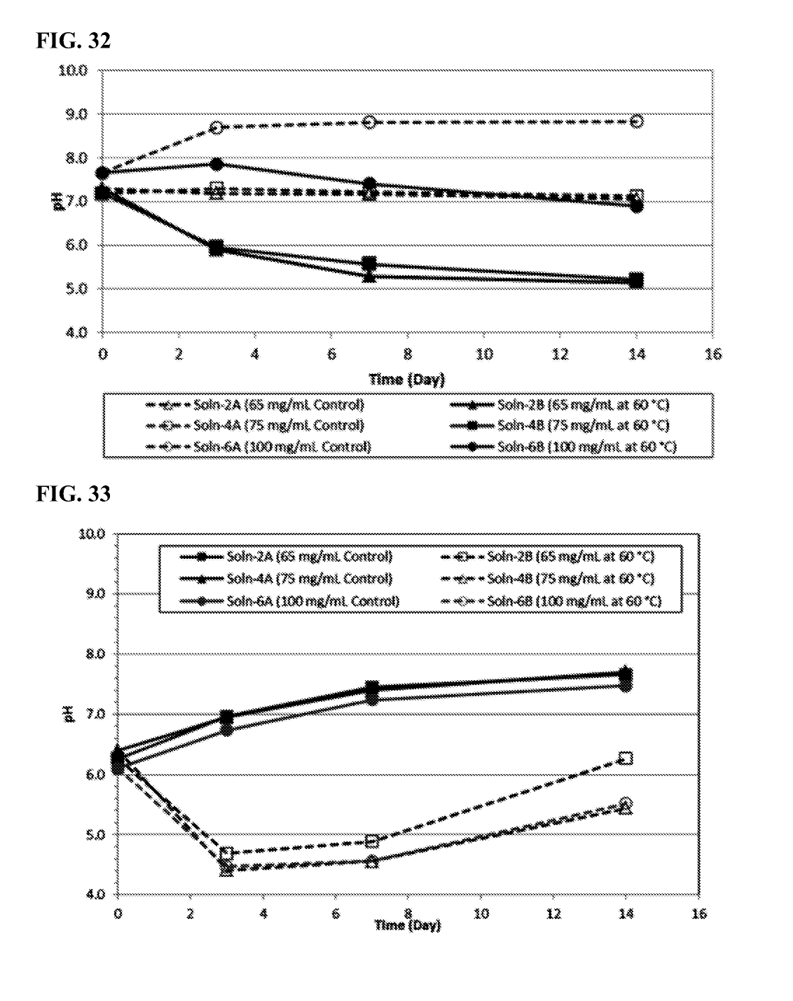

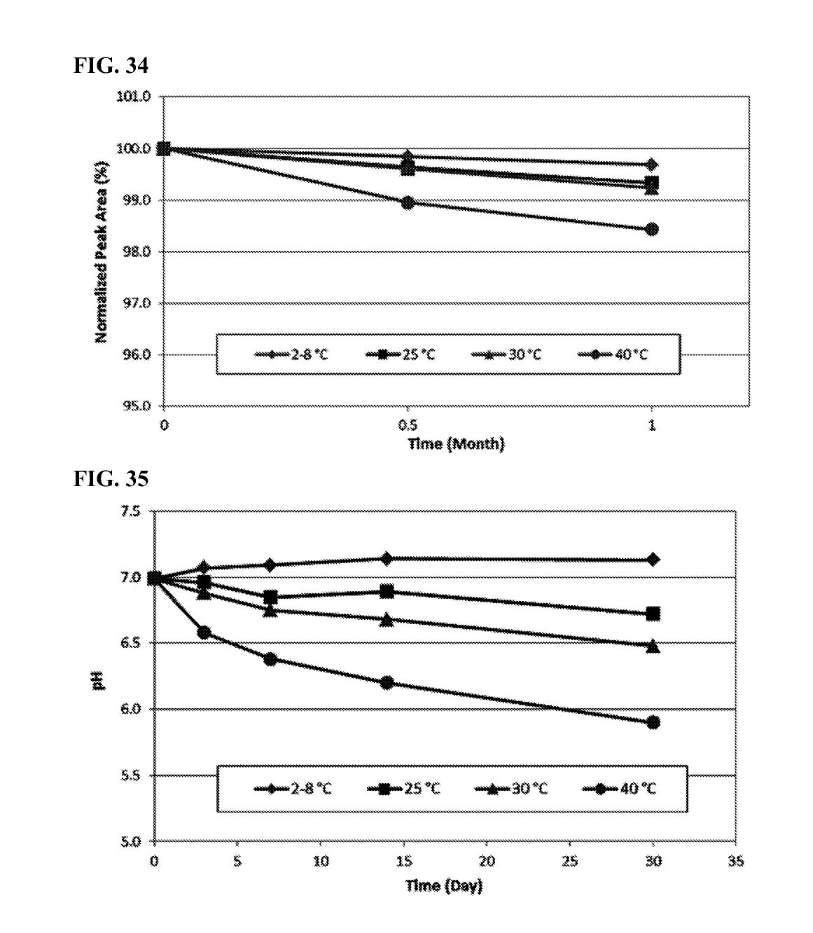

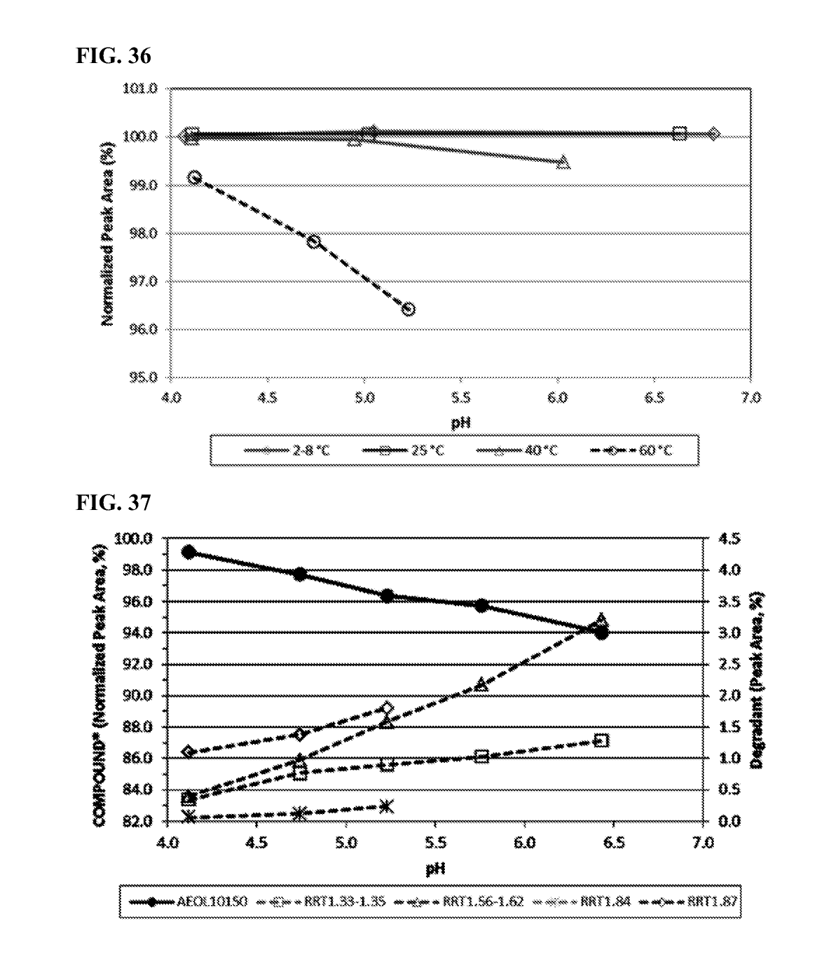

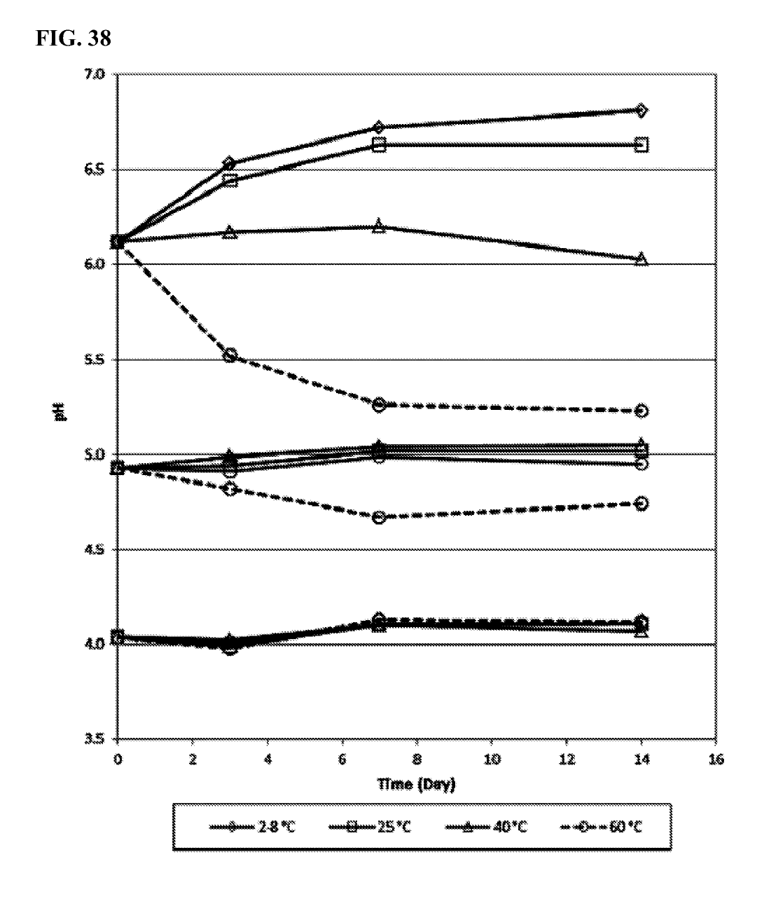

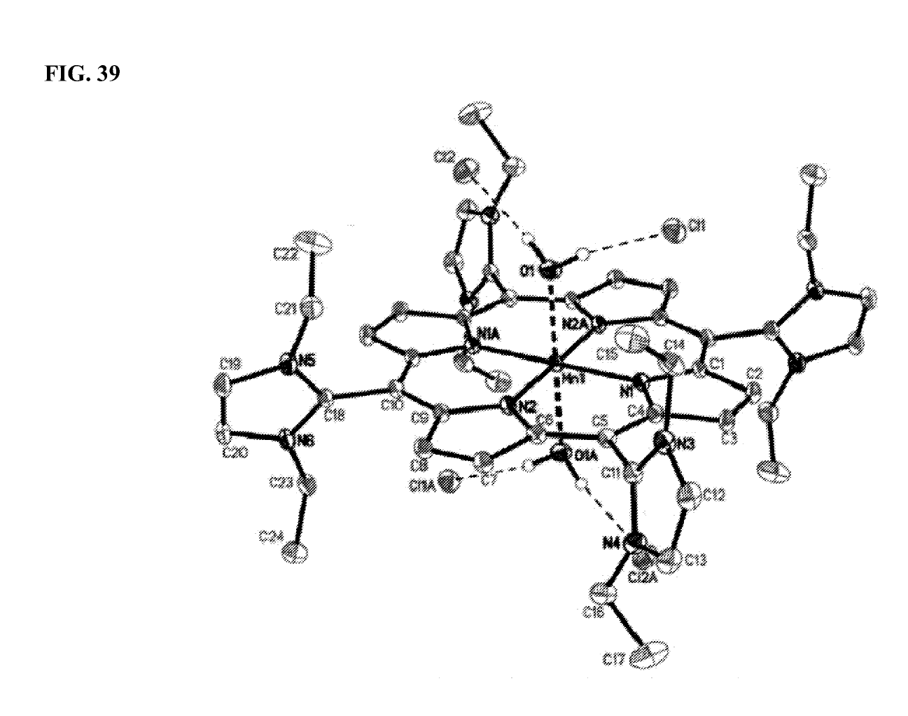

The results of UV-vis studies in the degassed water-0.1% TFA (FIG. 23) show that the band pattern characteristic for the reduced form compound (VI) (424 nm) which, upon air oxidation converts to the bands associated with the oxidized form of compound (VI) (446 nm).

A 12 L RBF was placed in a heating mantel and fitted with an overhead mechanical stirrer, nitrogen inlet and temperature probe connected to a J-CHEM™ controller. Porphyrin hexafluorophosphate (100 g), manganese (III) acetate (39.51 g) and acetonitrile (3250 mL) were charged into the reactor agitating at 320 RPMs. The reaction mixture was stirred at 40° C. for 7.5 hours until completion was observed by HPLC. After reaction completion the reaction mixture was stirred for an additional (for a minimum of) 4 hours at 40° C. then was allowed to attain the ambient temperature. At this time the solution of tetrabutylammonium chloride was prepared: tetrabutylammonium chloride (300 g) was dissolved in acetonitrile (1000 mL) and filtered through a 0.2 ˜L syringe filtering cartridge and set aside.

The content of the reaction flask was then filtered via a 0.2 micron filtering cartridge directly into a 12 L RBF that was fitted with an overhead mechanical stirrer and nitrogen inlet. Into that flask was added the pre-filtered tetrabutylammonium chloride/acetonitrile solution. After 20 minutes agitation the agitated slurry was filtered into a funnel that uses a 5 micron nylon filter cloth. Wash twice with 250 mL of acetone. Set to dry at 20° C. under a vacuum oven at constant weight. The isolated yield was 87.1 g. Air exposure of the product solution in 0.1% TFA in water results in only negligible changes in the UV-vis spectra indicating only minimal presence of Mn(II) species.

Sample Preparation. The sample consisted of dry, dark brown, almost completely opaque blocks. The crystal chosen for data collection was a brown block with the dimensions 0.15×0.17×0.20 mm³.

Data Collection and Data Reduction. The crystal was mounted with mineral oil (STP® Oil Treatment) on a MITEGEN™ mount. Diffraction data (ψ and ω-scans) were collected at 100K on a Bruker-AXS X8 Kappa Duo diffractometer coupled to a Smart Apex2 CCD detector with graphite-monochromated Mo Ka radiation (λ=0.71073 A) from a fine-focus sealed tube. Data reduction was carried out with the program SAINT¹ and semi-empirical absorption correction based on equivalents was performed with the program SADABSL². A summary of crystal properties and data/refinement statistics is given in Table 6.1.

TABLE 6.1
refinement data

| Identication code | sfy12 | |
|---|---|---|
| Empirical formula | C₄₈H₈₀Cl₅MnN₁₂O₁₄ | |
| Formula weight | 1281.43 | |
| Temperature | 100(2) K | |
| Wavelength | 0.71073 Å | |
| Crystal system | Monoclinic | |
| Space group | P2₁/c | |
| Unit cell dimensions | a = 13.396(4) Å | α = 90°. |
| | b = 14.885(4) Å | β = 107.175(4)°. |
| | c = 16.176(4) Å | γ = 90°. |

| | |
|---|---|
| Volume | 3081.5(14) Å³ |
| Z | 2 |
| Density (calculated) | 1.381 Mg/m³ |
| Absorption coefficient | 0.500 mm⁻¹ |
| F(000) | 1348 |
| Crystal size | 0.20 × 0.17 × 0.15 mm³ |
| Theta range for data collection | 1.59 to 30.51°. |
| Index ranges | −19 <= h <= 19, −21 <= k <= 21, |
| | −23 < = l <= 23 |
| Reflections collected | 139304 |
| Independent reflections | 9413 [Rᵢₙₜ = 0.0370] |
| Completeness to theta = 30.51° | 100.0% |
| Absorption correction | Semi-empirical from equivalents |
| Max. and min. transmission | 0.9288 and 0.9066 |
| Refinement method | Full-matrix least-squares on F² |
| Data/restraint/parameters | 9413/82/446 |
| Goodness-of-fit on F² | 1.066 |
| Final R indices [I > 2σ(I)] | R1 = 0.0358, wR2 = 0.0961 |
| R indices (all data) | R1 = 0.0422, wR2 = 0.1011 |
| Largest diff. peak and hole | 0.578 and −0.807 e · Å⁻³ |

Structure Solution and Refinement. The structure was solved with direct methods using the program SHELXS³ and refined against F² on all data with SHELXL⁴ using established refinement techniques⁵. All non-hydrogen atoms were refined anisotropically. Hydrogen atoms attached lo carbon atoms were placed in geometrically calculated positions and refilled using a riding model while constraining their Uᵢₛₒ to 1.2 times the U_{eq} of the atoms to which they bind (1.5 times for methyl groups). Coordinates for oxygen-bound hydrogen atoms were taken from the difference Fourier synthesis and all O-bound hydrogen atoms were refined semi-freely with the help of O—H distance restraints (target value 0.84(2) Å), while constraining their Uᵢₛₒ to 1.5 times the U_{eq} of the corresponding oxygen atoms. In addition, similarity restraints were used for H—O—H angles. For three of the water positions, namely O7 (50% occupancy), O8A (ca. 33% occupancy) and O8B (ca. 17% occupancy) no suitable hydrogen coordinates could be found. Those three partially occupied water molecules were refined as free oxygen atoms. All oxygen-bound hydrogen atoms are involved in reasonable hydrogen bonds (see Table 6.2).

TABLE 6.2
hydrogen bonds of crystal structure

| D—H . . . A | d(D—H) | d(H . . . A) | d(D . . . A) | <(DHA) |
|---|---|---|---|---|
| O(1)—H(1A) . . . Cl(2) | 0.840(9) | 2.189(9) | 3.0223(13) | 171.5(18) |
| O(1)—H(1B) . . . Cl(1) | 0.839(9) | 2.220(9) | 3.0470(12) | 169.0(18) |
| O(2)—H(2A) . . . Cl(2) | 0.822(9) | 2.363(12) | 3.1573(13) | 163(2) |
| O(2)—H(2B) . . . Cl(2)#2 | 0.823(9) | 2.350(11) | 3.1596(13) | 168(2) |
| O(3)—H(3A) . . . O(2) | 0.806(9) | 1.993(10) | 2.7852(18) | 167(2) |
| O(3)—H(3B) . . . O(8B) | 0.783(9) | 1.93(2) | 2.694(19) | 166(2) |
| O(3)—H(3B) . . . O(7) | 0.783(9) | 2.23(2) | 2.758(4) | 125(2) |
| O(3)—H(3B) . . . Cl(3) | 0.783(9) | 2.483(12) | 3.235(3) | 162(2) |
| O(4)—H(4A) . . . O(3)#3 | 0.817(9) | 2.040(10) | 2.8563(17) | 177(2) |
| O(4)—H(4B) . . . O(6) | 0.823(9) | 1.876(11) | 2.681(3) | 165(3) |
| O(4)—H(4B) . . . O(6A) | 0.823(9) | 2.035(11) | 2.857(5) | 176(2) |
| O(5)—H(5A) . . . Cl(1)#4 | 0.836(10) | 2.285(12) | 3.119(5) | 175(5) |
| O(5)—H(5B) . . . O(5)#5 | 0.831(10) | 2.177(16) | 3.004(12) | 174(5) |
| O(5A)—H(5D) . . . Cl(1)#4 | 0.836(10) | 2.343(14) | 3.170(5) | 170(5) |
| O(5A)—H(5C) . . . O(4) | 0.836(10) | 1.99(2) | 2.791(6) | 160(5) |
| O(6)—H(6A) . . . Cl(1)#1 | 0.833(10) | 2.357(11) | 3.183(3) | 171(4) |
| O(6)—H(6B) . . . Cl(3) | 0.834(10) | 2.238(11) | 3.068(3) | 174(4) |
| O(6A)—H(6C) . . . Cl(1)#1 | 0.833(10) | 2.356(12) | 3.187(4) | 175(5) |
| O(6A)—H(6D) . . . O(8A) | 0.835(10) | 2.12(3) | 2.837(9) | 144(5) |

Symmetry transformations used to generate equivalent atoms:
#1 −x + 1, −y + 1, −z + 1
#2 −x + 2, −y + 1, −z + 1
#3 −x + 2, −y + 1, −z + 2
#4 x, y, z + 1
#5 −x + 1, −y + 1, −z + 2

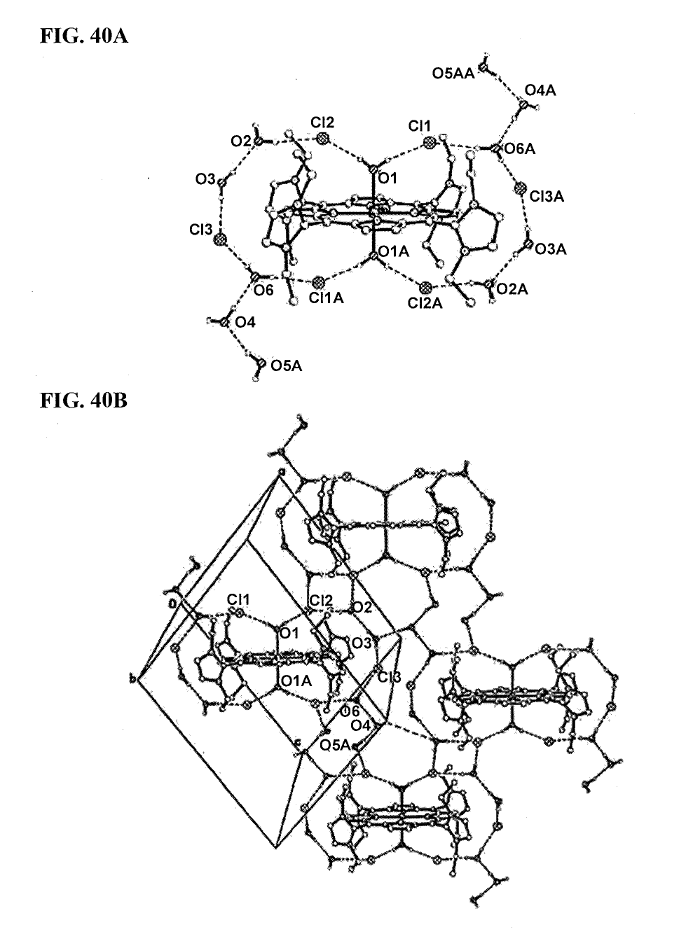

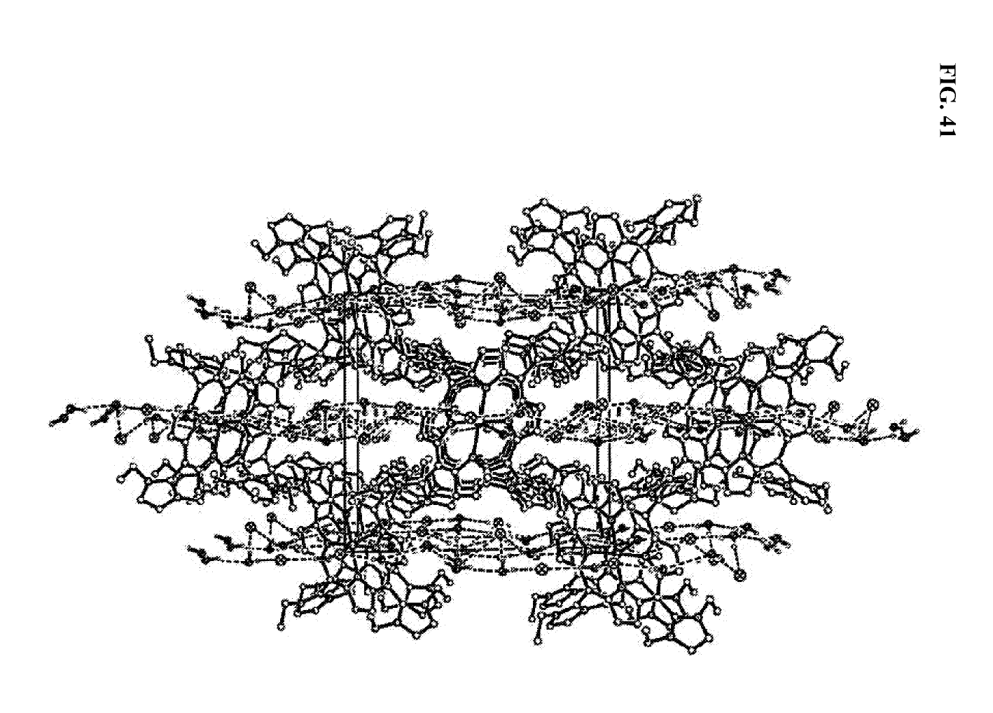

Crystal Structure. The submitted compound crystallizes in the centrosymmetric monoclinic space group P2₁c . The asymmetric unit contains half a target molecule, 2.5 chlorine ions and seven water molecules distributed over 11 sites. The manganese atom resides on the crystallographic inversion center and is coordinated by the four porphyrin nitrogen atoms in a square planar fashion. Completing the octahedral coordination sphere, a water molecule (0 1) and its symmetry equivalent are coordinated to the manganese in the two axial positions from above and below the porphyrin plane. The Mn1-O1 distance is 2.1760(10) Å, which corresponds to a bond order of 0.33⁶. In addition to this for a coordinating bond fairly strong interaction, this water molecule makes two strong O—H . . . CI hydrogen bonds to the two fully occupied chlorine atoms, Cl1 and Cl2, thus further fixating the water molecule. FIGS. 40A-40B show the full target molecule with atomic labeling scheme and the two mentioned O—H . . . Cl hydrogen bonds; Tables 6.2 and 6.3 give all hydrogen bonds and selected bond lengths and angles, respectively. In addition, the structure contains another crystallographically independent chlorine position, Cl3, which is half occupied. Together with water molecules O2, O3, O4, O5A and O6 (occupancies of the two disorder involved water molecules O5A and O6 is 59.3(4)% and 49.9(7)%), a two-dimensional sheet of O—H . . . Cl and O—R . . . O hydrogen bonds is formed, as illustrated in FIG. 41. Those sheets extend parallel to the a-c-plane and are stacked along the b-direction, repeating twice per unit cell (see FIG. 42). The other components of the disordered water molecules (O5 and O6A) are involved in slightly different hydrogen bonds that further stabilize the network.

As mentioned above, in addition to the six water molecules that form this hydrogen bond network, there are three additional water sites in the asymmetric unit to which no hydrogen atoms could be assigned. Those oxygen atoms are nevertheless involved in the hydrogen bonding insofar as they serve as hydrogen bond acceptors. Locating the water-hydrogen positions in the difference density map was simple and unequivocal for O1, O2 and O3. Hydrogen atoms on O4 could be located in the difference Fourier synthesis in plausible locations, however there were alternative positions which might also be possible, although less likely. Finding the hydrogen positions on the disordered water molecules O5/O5A and O6/O6A was less straightforward and inference from surrounding hydrogen bond acceptors was taken into consideration to come up with a reasonable hydrogen model. All hydrogen bonds are listed in Table 6.2.

TABLE 6.3
selected bonds and angles

| | | | |
|---|---|---|---|
| O(1)—Mn(1) | 2.1760(10) | N(1)#1—Mn(1)—N(1) | 180.0 |
| Mn(1)—N(1) | 2.0108(11) | N(1)#1—Mn(1)—N(2)#1 | 89.45(4) |
| Mn(1)—N(2) | 2.0202(11) | N(1)—Mn(1)—N(2)#1 | 90.55(4) |
| N(1)—C(4) | 1.3704(15) | N(1)#1—Mn(1)—N(2) | 90.55(4) |
| N(1)—C(1) | 1.3715(15) | N(1)—Mn(1)—N(2) | 89.45(4) |
| N(2)—C(9) | 1.3724(15) | N(2)#1—Mn(1)—N(2) | 180.0 |
| N(2)—C(6) | 1.3732(15) | N(1)#1—Mn(1)—O(1)#1 | 90.81(4) |
| C(1)—C(10)#1 | 1.3942(16) | N(1)—Mn(1)—O(1)#1 | 89.19(4) |
| C(1)—C(2) | 1.4386(17) | N(2)#1—Mn(1)—O(1)#1 | 89.09(4) |
| C(2)—C(3) | 1.3543(17) | N(2)—Mn(1)—O(1)#1 | 90.91(4) |
| C(3)—C(4) | 1.4399(16) | N(1)#1—Mn(1)—O(1) | 89.19(4) |
| C(4)—C(5) | 1.3944(16) | N(1)—Mn(1)—O(1) | 90.81(4) |
| C(5)—C(6) | 1.3925(16) | N(2)#1—Mn(1)—O(1) | 90.91(4) |
| C(5)—C(11) | 1.4750(16) | N(2)—Mn(1)—O(1) | 89.09(4) |
| C(6)—C(7) | 1.4398(16) | O(1)#1—Mn(1)—O(1) | 180.0 |
| C(7)—C(8) | 1.3566(17) | | |
| C(8)—C(9) | 1.4410(16) | | |
| C(9)—C(10) | 1.3948(16) | | |
| C(10)—C(1)#1 | 1.3941(16) | | |
| C(10)—C(18) | 1.4737(16) | | |

Oxidation State of the Manganese Atom. The model described above is supported by the assumption of an oxidation state of +HI of the central metal atom Mn 1. This is chemically reasonable, corresponds well to the color of the crystal, is in agreement with EPR spectra, and the electron count adds up as well: for each half Mn3+ ion, the asymmetric unit contains one half porphyrin ligand (the full ligand is two-fold positively charged, owing to the four singly positively charged substituents on the doubly negatively charged porphyrin ring) for a total of 2.5 positive charges in the asymmetric unit. This charge is perfectly balanced by the 2.5 chlorine atoms.

As mentioned above, the half occupied chlorine atom Cl3 is flanked by two low-occupancy oxygen atoms, O8A and O8B, and there is an additional half-occupied oxygen atom, O7. Those three positions add up to precisely one full oxygen atom, corresponding to 8 electrons, which is also approximately equivalent to one half chlorine ion. A model that spreads a full chloride ion over the four positions occupied by the above described positions for Cl3, O7, O8A and O8B is reasonably stable and gives rise to a good refinement statistic. Such a model is charge balanced assuming Mn(IV), as the asymmetric unit would then contain three full Cl⁻ ions instead of 2.5. The refinement of the Mn(IV) model is slightly less stable than that of the one assuming Mn(III) and it seems therefore likely that the metal is indeed present in form of a Mn³⁺ ion.

Possibility of Fewer Chlorine Ions. It has been reported that the compound at hand may, over time, eliminate HCl. This suggests that the structure at hand may contain fewer than five Cl⁻ ions per Mn atom. As described above, a model with more than five Cl⁻ ions (namely six) is reasonable, although unlikely. A model with fewer than five chloride ions, on the other hand, is not reasonable based on the diffraction data at hand The two chloride ions Cl1 and Cl2 are connected to the target molecule by means of fairly strong hydrogen bonds and their thermal parameters are relatively small, suggesting that those sites would not be satisfied with fewer electrons than those of a chloride ion. The remaining chlorine atom, Cl3, is only half occupied and two low-occupancy water molecules (O8A and O8B) are situated on either side of Cl3. A model that refines those three positions as one fully occupied water molecule distributed over three sites results in negative Uᵢₛₒ values for the three water positions, indicating that the eight electrons of an oxygen atom are not enough for this site. Refining the occupancy of Cl3 and O8A/O8B freely (while constraining their sum to unity to allow for no more than one atom to reside in that one place) results in 43.1 (3)% chlorine and 56.9(3)% water (that water, of course, distributed over two sites), which is quite close to the model containing exactly 50% chlorine in that position.

Therefore, the lowest number of chloride ions per manganese reasonably supported by the diffraction data at hand is 4.85. It is certainly conceivable that, over time, some or all of Cl3 could disappear while the analyzed crystal still had it in place. This would result in a void in the crystal lattice which may not be destabilizing enough to lead to a breakdown of the lattice, especially if the void could be filled in with water from the outside (see below). Most probably Cl would disappear as HCl, which means that half a hydrogen atom would have to disappear from the asymmetric unit over time. It is fair to assume that such a hydrogen atom should make a hydrogen bond to Cl3 in the structure at hand Only two hydrogen atoms are potential candidates, one each on O3 and O6/O6A (see FIG. 40A). It would seem likely that any disappearing chlorine would take a hydrogen atom from one of those positions with it, thus rationalizing the observation of HCl elimination.

Possibility of Fewer or More Water Molecules. It has been reported that the compound, in its crystalline state, can reversibly absorb and release significant amounts of water. The crystal structure at hand contains 14 water molecules for every Mn atom. Water molecules O1 to O6 are fully occupied (although O5 and O6 are disordered over two positions) and there is no indication that any of those six positions could be modeled successfully in significantly reduced occupancy. Such an indication would be significantly higher than average thermal parameters of an oxygen atom. Of the fully occupied water molecules, only O5/O5A shows somewhat Larger thermal parameters, but not to an extent that would suggest reduced occupancy. Water O7 is half occupied and shows fairly large thermal parameters, suggesting it may possibly be slightly less than half an oxygen atom, but certainly not more much less than half. That means the crystal structure at band contains at least 13.5 to 14 water molecules per Mn. As mentioned above, the MnI—O1 distance is 2.1760(10) Å, which corresponds to a Bond Order of almost ⅓.

In addition, the hydrogen atoms on O1 are involved in two fairly strong hydrogen bonds with Cl1 and Cl2. This makes it seem unlikely that O1 would readily be extractable from the crystal, but it is conceivable that all water molecules except for O1 might leave the crystal lattice, possibly without significantly damaging the lattice's structural integrity, and be replaced at a later time. This would bring the possible water count down to two water molecules per Mn atom (in this case one negative charge would be missing, unless the half chloride Cl3 stays behind—it seems unlikely that O1 could be deprotonated). The question how much the crystal lattice would suffer from removal of all six crystallographically independent free water molecules is bard to answer, however it seems that a solvent-free model, based only on Mn1, the ligand, Cl1, Cl2 and the O1 water, still gives rise to a fairly compact packing. In any case, it is difficult to predict, which of the water molecules would disappear first. Probably the already half occupied O7 is a prime candidate and after that the disordered water molecules O5/O5A, O6/O6A and O8A/O8B might be most likely to follow, but this guess is difficult to substantiate without determining the crystal structure of a sample with low water content.

Another question of interest is whether the structure at hand provides space to accommodate additional water. The program PLATON⁷ was used to perform a void analysis, with the result that the structure does not contain any solvent accessible voids, not even large enough for a water molecule (a hydrogen bonded water molecule takes approximately 40 Å of space). The only possibility for additional water in the crystal structure at hand is the half-occupied water position O7. O7 is 4.97 Å away from its nearest own symmetry equivalent, which means there is no crystallographic reason for this site not to be fully occupied. Therefore the crystal structure at hand could easily accommodate 15 water molecules per Mn atom. If all of Cl3 were to disappear in the manner discussed above, and if it were to be replaced with water from the outside, the overall count could even be as high as 16 water molecules per Mn atom (even though one of those waters would have to be an OH. ion to keep the charge balanced—the missing hydrogen atom would have left with Cl3 in form of HCl). Thus, the crystal structure at hand conceivably supports the hypothesis that a crystal of this species could contain any amount of water between 2 and 16 water molecules per Mn atom. Certainly not more than 16 and most probably not fewer than 2, as those two waters that are directly bound to the Mn and are making strong hydrogen bonds to Cl1 and Cl2 are not likely to be removable, at least not with mild methods.

### REFERENCES

[1]. Bruker (2011). SAINT, Bruker-AXS Inc., Madison, Wis. USA; [2]. Shldrick, G. M. (2009). SADABS, Univ. of Gottingen, Germany; [3]. Sheldrick, G. M., Acta Cryst. 1990, A46, 467-473; [4]. Sheldrick, G. M., Acta Cryst. 2008, A64, 112-122; [5]. Muller, P., Crystal. Rev. 2009, 15, 57-83; [6]. Breese, N. E. & O'Keefe, M., Acta Cryst., 1991, B47, 192-197; [7]. Spek, A. L., Acta Cryst. 2009, D65, 148-155.

1. A method for synthesizing a substituted porphyrin having the formula: 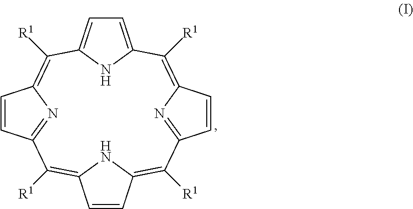
wherein R¹ is substituted or unsubstituted heterocycloalkyl or substituted or unsubstituted heteroaryl, said method comprising:
(i) contacting a pyrrole with an R¹-substituted aldehyde, wherein said contacting is performed in a solvent system comprising a positive azeotrope;
(ii) allowing said pyrrole to react with said R¹-substituted aldehyde in said solvent system under azeotropic distillation conditions, thereby forming a substituted-porphyrinogen; and
(iii) oxidizing said substituted-porphyrinogen, thereby synthesizing a substituted porphyrin having formula (I).

2.-5. (canceled)

6. The method of claim 1, wherein R¹ is:

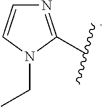 [SMILES: *c1nccn1CC]

7.-33. (canceled)

34. A method for synthesizing a compound of formula 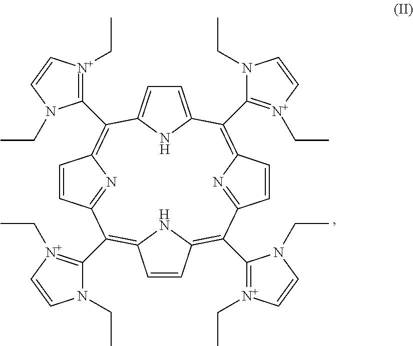 [SMILES: CCn1cc[n+](CC)c1-c1c2nc(c(-c3n(CC)cc[n+]3CC)c3ccc([nH]3)c(-c3n(CC)cc[n+]3CC)c3nc(c(-c4n(CC)cc[n+]4CC)c4ccc1[nH]4)C=C3)C=C2]
said method comprising:
contacting with an ethylating agent a compound having the formula 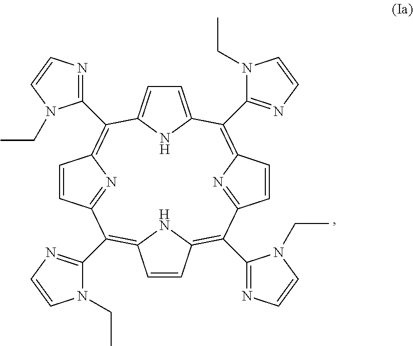 [SMILES: CCn1ccnc1-c1c2nc(c(-c3nccn3CC)c3ccc([nH]3)c(-c3nccn3CC)c3nc(c(-c4nccn4CC)c4ccc1[nH]4)C=C3)C=C2]
thereby synthesizing a compound of formula (II).

35. (canceled)

36. The method of claim 34, wherein said method further comprises:
(i) contacting about one equivalent of a pyrrole with about one equivalent of 1-ethyl-1H-imidazole-2-carbaldehyde, wherein said contacting is performed in a solvent comprising a positive azeotrope;
(ii) allowing said pyrrole to react with said 1-ethyl-1H-imidazole-2-carbaldehyde, in said solvent under azeotropic distillation conditions, thereby forming a substituted-porphyrinogen; and
(iii) oxidizing said substituted-porphyrinogen, thereby synthesizing a substituted porphyrin having formula (Ia).

37.-44. (canceled)

45. The method of claim 34, wherein said method further comprises precipitation of the compound having formula (II) with a precipitating agent.

46. (canceled)

47. The method of claim 34, wherein said method further includes contacting the compound of formula (II) with a metal salt.

48. (canceled)

49. (canceled)

50. A method for synthesizing a hydrate compound having the formula 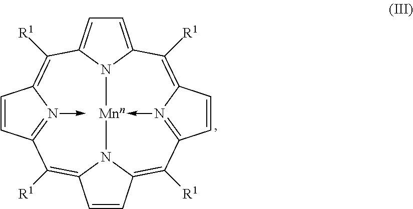
wherein R¹ is substituted or unsubstituted heterocycloalkyl or substituted or unsubstituted heteroaryl; and n is 2 or 3, said method comprising:
(i) contacting a compound of formula 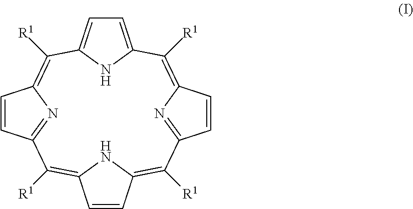
with over about 2 equivalents of a Mn(III) salt in a solvent, thereby forming a reaction mixture;
(ii) heating said reaction mixture thereby synthesizing a compound of formula (III); and
(iii) hydrating said compound of formula (III) thereby forming a hydrate of compound (III).

51. The method of claim 50, wherein R¹ is substituted or unsubstituted imidazolyl, substituted or unsubstituted pyrazolyl, substituted or unsubstituted thiazolyl, or substituted or unsubstituted triazolyl.

52.-69. (canceled)

70. A container comprising a plurality compounds, wherein said plurality of compounds have the formula:

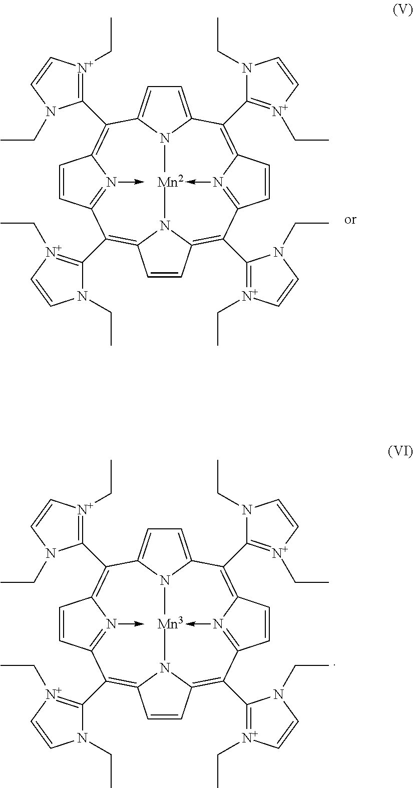

71.-73. (canceled)

74. The container of claim 70, further comprising a counterion selected from the group consisting of a halogen anion, SCN⁻, HSO₄⁻, SO₄⁻², H₂PO₄⁻¹, HPO₄⁻², PO₄⁻³, NO₃⁻, PF₆⁻, or BF₄⁻.

75.-80. (canceled)

81. A pharmaceutical formulation comprising water and a compound having the formula:

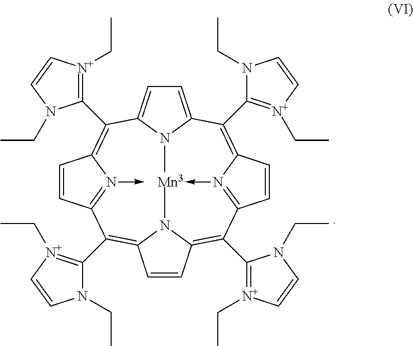

82. The pharmaceutical formulation of claim 81, wherein the formulation comprises less than 10% Mn(II).

83.-89. (canceled)

90. A method for purifying a compound of formula: 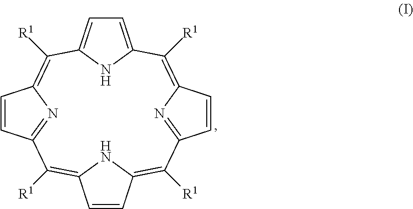
said method comprising:
(i) combining a compound of formula (I) and a purification solvent in a reaction vessel thereby forming a purification mixture, wherein said compound is insoluble in said purification solvent;
(ii) heating said purification mixture;
(iii) cooling said purification mixture; and
(iv) filtering said purification mixture thereby purifying a compound of formula (I).

91. The method of claim 90, wherein said purification solvent is 2-butanone, 1,4-dioxane, acetonitrile, ethyl acetate or cyclohexanone.

92.-98. (canceled)

99. A method for purifying a compound having the formula: 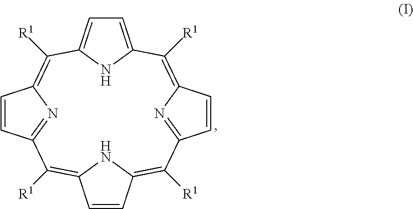
wherein, said method comprises:
(i) dissolving a compound of formula (I) in a purifying solvent in a reaction vessel to form a purifying mixture;
(ii) heating said purifying mixture;
(iii) cooling said purifying mixture; and
(iv) drying said purifying mixture thereby purifying a compound of formula (I).

100.-105. (canceled)

106. A crystal comprising a compound having the formula:

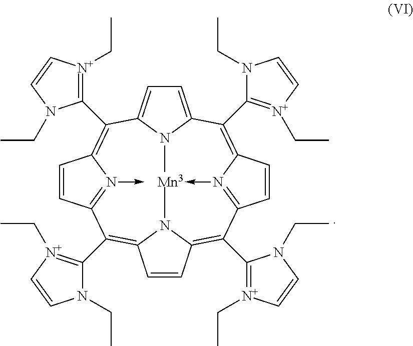

107. (canceled)

108. A crystalline form of [5,10,15,20-tetrakis(1,3-diethylimidazolium-2-yl)porphyrinato]manganese(III) chloride hydrate complex characterized by an x-ray powder diffraction spectrum,
said x-ray powder diffraction spectrum comprising angle 2θ peaks at about 6.9±0.2, 8.2±0.2, 9.5±0.2, 11.4±0.2, 12.8±0.2, 14.5±0.2, 15.0±0.2, 16.1±0.2, 16.3±0.2, 18.1±0.2, 20.3±0.2, 23.5±0.2, 24.8±0.2, 25.6±0.2, 26.5±0.2, and 29.2±0.2, or said x-ray powder diffraction spectrum comprising angle 2θ peaks at about 26.2±0.2, 22.9±0.2, 20.0±0.2, 18.6±0.2, 15.2±0.2, 13.7±0.2, 13.5±0.2, 13.0±0.2, 12.4±0.2, 11.4±0.2, 10.6±0.2, 8.9±0.2, 6.8±0.2, and 6.0±0.2, or
said x-ray powder diffraction spectrum comprising angle 2θ peaks at about 27.7±0.2, 26.6±0.2, 19.9±0.2, 15.4±0.2, 14.7±0.2, 11.6±0.2, 10.1±0.2, 8.6±0.2, and 6.9±0.2, or
said x-ray powder diffraction spectrum comprising angle 2θ peaks at about 29.5±0.2, 27.3±0.2, 26.3±0.2, 24.7±0.2, 23.5±0.2, 22.5±0.2, 21.6±0.2, 20.5±0.2, 19.3±0.2, 17.7±0.2, 13.1±0.2, 10.8±0.2, 9.9±0.2, 8.5±0.2, and 6.0±0.2, or
said x-ray powder diffraction spectrum comprising angle 2θ peaks at about 23.5±0.2, 9.1±0.2, 6.9±0.2, and 5.8±0.2, or
said x-ray powder diffraction spectrum comprising angle 2θ peaks at about 27.7±0.2, 23.6±0.2, 23.1±0.2, 20.7±0.2, 6.9±0.2, and 5.8±0.2, or
said x-ray powder diffraction spectrum comprising angle N peaks at about 27.7±0.2, 20.7±0.2, 13.8±0.2, 11.4±0.2, 9.5±0.2, 8.2±0.2, and 6.9±0.2,
wherein said an x-ray powder diffraction spectrum is obtained using a Cu Kα radiation source (1.54 Å).

109. The crystalline form of 108, wherein
said x-ray powder diffraction spectrum further comprises angle 2θ peaks at about 13.8±0.2, 17.4±0.2, 19.0±0.2, 19.4±0.2, 20.7±0.2, 21.1±0.2, 21.5±0.2, 22.0±0.2, 22.5±0.2, 22.8±0.2, 26.9±0.2, 27.6±0.2, 28.5±0.2, 30.2±0.2, 30.5±0.2, 31.2±0.2, 37.3±0.2, 38.5±0.2, and 41.1±0.2, or
said x-ray powder diffraction spectrum further comprises angle 2θ peaks at about 29.4±0.2, 28.5±0.2, 27.5±0.2, 27.0±0.2, 25.7±0.2, 25.2±0.2, 23.7±0.2, 17.8±0.2, 17.1±0.2, 14.6±0.2, 10.9±0.2, 9.9±0.2, and 8.2±0.2, or
said x-ray powder diffraction spectrum further comprises angle 2θ peaks at about 29.6±0.2, 25.7±0.2, 23.4±0.2, 20.4±0.2, and 13.7±0.2, or
said x-ray powder diffraction spectrum further comprises angle 2θ peaks at about 32.6±0.2, 19.8±0.2, 18.6±0.2, and 14.8±0.2, or
said x-ray powder diffraction spectrum further comprises angle 2θ peaks at about 27.5±0.2, 24.6±0.2, 18.2±0.2, 13.9±0.2, 13.0±0.2, 11.7±0.2, and 7.9±0.2, or
said x-ray powder diffraction spectrum further comprises angle 2θ peaks at about 29.2±0.2, 28.9±0.2, 27.1±0.2, 26.5±0.2, 26.2±0.2, 24.8±0.2, 22.4±0.2, 22.2±0.2, 21.5±0.2, 20.3±0.2, 18.1±0.2, 17.3±0.2, 16.3±0.2, 14.9±0.2, 13.8±0.2, 11.5±0.2, and 9.2±0.2, or
said x-ray powder diffraction spectrum further comprises angle 2θ peaks at about 23.5±0.2, 22.8±0.2, 16.3±0.2, and 5.9±0.2.

110. A crystalline form of [5,10,15,20-tetrakis(1,3-diethylimidazolium-2-yl)porphyrinato]manganese(III) chloride hydrate complex characterized by an x-ray powder diffraction spectrum,
said x-ray powder diffraction spectrum comprising d spacings at about 12.85, 10.82, 9.28, 7.78, 6.91, 6.11, 5.91, 5.49, 5.42, 4.89, 4.37, 3.78, 3.58, 3.47, 3.36, and 3.06, or
said x-ray powder diffraction spectrum comprising d spacings at about 14.74, 12.93, 9.99, 8.34, 7.74, 7.14, 6.80, 6.55, 6.45, 5.83, 4.78, 4.43, 3.89, and 3.40, or
said x-ray powder diffraction spectrum comprising d spacings at about 12.89, 10.27, 8.79, 7.60, 6.04, 5.74, 4.45, 3.35, and 3.22, or
said x-ray powder diffraction spectrum comprising d spacings at about 15.12, 12.74, 9.75, and 3.78, or
said x-ray powder diffraction spectrum comprising d spacings at about 12.84, 10.83, 9.26, 7.77, 6.43, 4.29, and 3.22,
wherein said an x-ray powder diffraction spectrum is obtained using a Cu Kα radiation source (1.54 Å).

111. The crystalline form of claim 110, wherein said x-ray powder diffraction spectrum further comprises d spacings at about, 7.57, 6.44, 5.10, 4.67, 4.58, 4.29, 4.2, 4.13, 4.05, 3.96, 3.89, 3.31, 3.22, 3.13, 2.96, 2.93, 2.86, 2.41, 2.34, and 2.19, or
said x-ray powder diffraction spectrum further comprises d spacings at about 10.82, 8.90, 8.10, 6.05, 5.19, 4.98, 3.75, 3.54, 3.47, 3.30, 3.24, 3.13, and 3.04, or
said x-ray powder diffraction spectrum further comprises d spacings at about 6.45, 4.35, 3.80, 3.46, and 3.02, or
said x-ray powder diffraction spectrum further comprises d spacings at about 11.14, 7.55, 6.81, 6.36, 4.87, 3.62, and 3.24, or
said x-ray powder diffraction spectrum further comprises d spacings at about 15.07, 12.84, 10.83, 9.26, 7.77, 6.43, 5.42, 4.29, 3.89, 3.79, and 3.22.

112.-131. (canceled)
